SEQUENCE LISTING (72 sequences)

SEQ ID NO: 1 - protein, 805 aa, synthetic construct [sequence omitted]

SEQ ID NO: 2 - protein, 723 aa, synthetic construct
  source 1..723: note = Q18-S740 of Human wild type ACE2
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 598 aa, synthetic construct
  source 1..598: note = Q18-D615 of Human wild type ACE2
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 4 - protein, 723 aa, synthetic construct
  source 1..723: note = knock out variant of Q18-S740 of Human wild type ACE2
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 5 - protein, 112 aa, synthetic construct
  source 1..112: note = IgM CH2 domain
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVP

SEQ ID NO: 6 - protein, 106 aa, synthetic construct
  source 1..106: note = IgM CH3 domain
DQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPK

SEQ ID NO: 7 - protein, 112 aa, synthetic construct
  source 1..112: note = IgM CH4 domain
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTG

SEQ ID NO: 8 - protein, 131 aa, synthetic construct
  source 1..131: note = IgM CH4 domain includes tailpiece sequence
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 9 - protein, 19 aa, synthetic construct
  source 1..19: note = IgM CH4 domain tailpiece sequence
KPTLYNVSLVMSDTAGTCY

SEQ ID NO: 10 - protein, 152 aa, synthetic construct
  source 1..152: note = J-Chain variant
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPDGGGGSEQKLISEEDL

SEQ ID NO: 11 - DNA, 456 bp, synthetic construct
  source 1..456: mol_type = other DNA; note = J-Chain variant
caggaggacgagaggatcgtgctggtggataacaagtgcaagtgtgcccggatcacatctaggatcatccgcagctccgaggaccctaacgaggatatcgtggagcggaatatcagaatcatcgtgccactgaacaatagagagaatatctctgaccccaccagccctctgcggacaagattcgtgtaccacctgtccgacctgtgcaagaagtgtgatccaaccgaggtggagctggacaaccagatcgtgaccgccacacagagcaatatctgcgacgaggattccgccaccgagacatgctacacctatgatcggaacaagtgttacacagccgtggtgcccctggtgtatggaggagagacaaagatggtggagaccgccctgacaccagacgcatgttatccagatggaggaggaggctccgagcagaagctgatctctgaggaggatctg

SEQ ID NO: 12 - protein, 137 aa, synthetic construct
  source 1..137: note = J-Chain variant
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 13 - DNA, 411 bp, synthetic construct
  source 1..411: mol_type = other DNA; note = J-Chain variant
caggaggacgagaggatcgtgctggtggataacaagtgcaagtgtgcccggatcacaagcaggatcatccgcagctccgaggaccctaacgaggatatcgtggagcggaatatcagaatcatcgtgccactgaacaatagagagaatatctctgaccccaccagccctctgcggacaagattcgtgtaccacctgtccgacctgtgcaagaagtgtgatccaaccgaggtggagctggacaaccagatcgtgaccgccacacagtccaatatctgcgacgaggattctgccaccgagacatgctacacctatgatcggaacaagtgttacacagccgtggtgcccctggtgtatggaggagagacaaagatggtggagaccgccctgacaccagacgcctgttatcccgat

SEQ ID NO: 14 - protein, 969 aa, synthetic construct
  source 1..969: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSEQKLISEEDL

SEQ ID NO: 15 - DNA, 2907 bp, synthetic construct
  source 1..2907: mol_type = other DNA; note = Chain H1 variant
cagtccacaatcgaggagcaggccaagaccttcctggacaagtttaaccacgaggccgaggatctgttctatcagagctccctggccagctggaactacaataccaacatcacagaggagaatgtgcagaacatgaacaatgccggggacaagtggtccgcctttctgaaggagcagtctacactggcccagatgtaccctctgcaggagatccagaacctgaccgtgaagctgcagctgcaggccctgcagcagaatggctctagcgtgctgtctgaggataagagcaagcggctgaatacaatcctgaacaccatgtccacaatctattctaccggcaaggtgtgcaatcccgacaaccctcaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactacgaggtgaacggcgtggacggctacgattattccagaggccagctgatcgaggatgtggagcacacattcgaggagatcaagccactgtatgagcacctgcacgcctacgtgagggccaagctgatgaacgcctatccttcttacatcagcccaatcggatgcctgcccgcccacctgctgggcgacatgtggggccgcttctggacaaacctgtacagcctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccccggcaacgtgcagaaggccgtgtgccaccctacagcctgggacctgggcaagggcgatttcagaatcctgatgtgcaccaaggtgacaatggacgattttctgaccgcccaccacgagatgggccacatccagtatgatatggcatacgcagcacagcccttcctgctgaggaatggagccaacgagggctttcacgaggccgtgggcgagatcatgtctctgagcgccgccacaccaaagcacctgaagtccatcggcctgctgtctcccgacttccaggaggataacgagaccgagatcaattttctgctgaagcaggccctgaccatcgtgggcacactgcccttcacctatatgctggagaagtggagatggatggtgtttaagggcgagatccctaaggaccagtggatgaagaagtggtgggagatgaagagggagatcgtgggagtggtggagcccgtgcctcacgacgagacctactgtgatcccgcctccctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgcgcctgggcaagagcgagccatggaccctggccctggagaacgtggtgggagccaagaatatgaacgtgcggcccctgctgaattatttcgagcctctgtttacatggctgaaggatcagaataagaacagctttgtgggctggtctaccgactggagcccctacgccgatcagtccatcaaggtgagaatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatcagctttaacttctttgtgacagccccaaagaacgtgagcgatatcatccccagaaccgaggtggagaaggccatccggatgagccggtccagaatcaacgacgccttccgcctgaatgataactccctggagtttctgggcatccagcctaccctgggcccacctaatcagccacccgtgagcgagccaaagagctccgacaagacccacacatgccctccatgtccggcgccagaggccgccggaggacctagcgtgttcctgtttccccctaagccaaaggatacactgatgatcagccggacaccagaggtgacctgcgtggtggtgtctgtgagccacgaggaccccgaggtgaagttcaactggtatgtggatggcgtggaggtgcacaatgccaagaccaagcctagagaggagcagtataacagcacatacagggtggtgtccgtgctgaccgtgctgcaccaggactggctgaacggcaaggagtacaagtgcaaggtgtctaataaggccctgcctgccccaatcgagaagaccatcagcaaggcaaagggacagcctcgcgaaccacaggtgtacgtgtatccaccctcccgggacgagctgacaaagaaccaggtgtctctgacctgtctggtgaagggcttttatccctctgatatcgccgtggagtgggagagcaatggccagcctgagaacaattacaagaccacacctccagtgctggacagcgatggctccttcgccctggtgagcaagctgacagtggacaagtccaggtggcagcagggcaacgtgttttcctgttctgtgatgcacgaggccctgcacaatcactacacccagaagagcctgagcttaagcccaggaggaggaggaggctctgagcagaagctgatcagcgaggaggatctg

SEQ ID NO: 16 - protein, 231 aa, synthetic construct
  source 1..231: note = Chain H2 variant
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 17 - DNA, 693 bp, synthetic construct
  source 1..693: mol_type = other DNA; note = Chain H2 variant
gagccaaagagctccgacaagacccacacatgccccccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgaaccacaggtctacgtgctgcctccatcccgggacgagctgacaaagaaccaggtctctctgctgtgcctggtgaaaggcttctatccatcagatattgctgtggagtgggaaagcaatgggcagcccgagaacaattacctgacttggccccctgtgctggactctgatgggagtttctttctgtattctaagctgaccgtggataaaagtaggtggcagcagggaaatgtctttagttgttcagtgatgcatgaagccctgcataaccactacacccagaaaagcctgtccctgtcccccgga

SEQ ID NO: 18 - protein, 969 aa, synthetic construct
  source 1..969: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGGGGGSEQKLISEEDL

SEQ ID NO: 19 - DNA, 2907 bp, synthetic construct
  source 1..2907: mol_type = other DNA; note = Chain H1 variant
cagtccacaatcgaggagcaggccaagaccttcctggacaagtttaaccacgaggccgaggatctgttctatcagagctccctggccagctggaactacaataccaacatcacagaggagaatgtgcagaacatgaacaatgccggggacaagtggtccgcctttctgaaggagcagtctacactggcccagatgtaccctctgcaggagatccagaacctgaccgtgaagctgcagctgcaggccctgcagcagaatggctctagcgtgctgtctgaggataagagcaagcggctgaatacaatcctgaacaccatgtccacaatctattctaccggcaaggtgtgcaatcccgacaaccctcaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactacgaggtgaacggcgtggacggctacgattattccagaggccagctgatcgaggatgtggagcacacattcgaggagatcaagccactgtatgagcacctgcacgcctacgtgagggccaagctgatgaacgcctatccttcttacatcagcccaatcggatgcctgcccgcccacctgctgggcgacatgtggggccgcttctggacaaacctgtacagcctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccccggcaacgtgcagaaggccgtgtgccaccctacagcctgggacctgggcaagggcgatttcagaatcctgatgtgcaccaaggtgacaatggacgattttctgaccgcccacaacgagatgggcaacatccagtatgatatggcatacgcagcacagcccttcctgctgaggaatggagccaacgagggctttcacgaggccgtgggcgagatcatgtctctgagcgccgccacaccaaagcacctgaagtccatcggcctgctgtctcccgacttccaggaggataacgagaccgagatcaattttctgctgaagcaggccctgaccatcgtgggcacactgcccttcacctatatgctggagaagtggagatggatggtgtttaagggcgagatccctaaggaccagtggatgaagaagtggtgggagatgaagagggagatcgtgggagtggtggagcccgtgcctcacgacgagacctactgtgatcccgcctccctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgcgcctgggcaagagcgagccatggaccctggccctggagaacgtggtgggagccaagaatatgaacgtgcggcccctgctgaattatttcgagcctctgtttacatggctgaaggatcagaataagaacagctttgtgggctggtctaccgactggagcccctacgccgatcagtccatcaaggtgagaatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatcagctttaacttctttgtgacagccccaaagaacgtgagcgatatcatccccagaaccgaggtggagaaggccatccggatgagccggtccagaatcaacgacgccttccgcctgaatgataactccctggagtttctgggcatccagcctaccctgggcccacctaatcagccacccgtgagcgagccaaagagctccgacaagacccacacatgccctccatgtccggcgccagaggccgccggaggacctagcgtgttcctgtttccccctaagccaaaggatacactgatgatcagccggacaccagaggtgacctgcgtggtggtgtctgtgagccacgaggaccccgaggtgaagttcaactggtatgtggatggcgtggaggtgcacaatgccaagaccaagcctagagaggagcagtataacagcacatacagggtggtgtccgtgctgaccgtgctgcaccaggactggctgaacggcaaggagtacaagtgcaaggtgtctaataaggccctgcctgccccaatcgagaagaccatcagcaaggcaaagggacagcctcgcgaaccacaggtgtacgtgtatccaccctcccgggacgagctgacaaagaaccaggtgtctctgacctgtctggtgaagggcttttatccctctgatatcgccgtggagtgggagagcaatggccagcctgagaacaattacaagaccacacctccagtgctggacagcgatggctccttcgccctggtgagcaagctgacagtggacaagtccaggtggcagcagggcaacgtgttttcctgttctgtgatgcacgaggccctgcacaatcactacacccagaagagcctgagcttaagcccaggaggaggaggaggctctgagcagaagctgatcagcgaggaggatctg

SEQ ID NO: 20 - protein, 954 aa, synthetic construct
  source 1..954: note = Chain H2 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 21 - DNA, 2862 bp, synthetic construct
  source 1..2862: mol_type = other DNA; note = Chain H2 variant
cagtccacaatcgaggagcaggccaagaccttcctggacaagtttaaccacgaggccgaggatctgttctatcagagctccctggccagctggaactacaataccaacatcacagaggagaatgtgcagaacatgaacaatgccggggacaagtggtccgcctttctgaaggagcagtctacactggcccagatgtaccctctgcaggagatccagaacctgaccgtgaagctgcagctgcaggccctgcagcagaatggctctagcgtgctgtctgaggataagagcaagcggctgaatacaatcctgaacaccatgtccacaatctattctaccggcaaggtgtgcaatcccgacaaccctcaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactacgaggtgaacggcgtggacggctacgattattccagaggccagctgatcgaggatgtggagcacacattcgaggagatcaagccactgtatgagcacctgcacgcctacgtgagggccaagctgatgaacgcctatccttcttacatcagcccaatcggatgcctgcccgcccacctgctgggcgacatgtggggccgcttctggacaaacctgtacagcctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccccggcaacgtgcagaaggccgtgtgccaccctacagcctgggacctgggcaagggcgatttcagaatcctgatgtgcaccaaggtgacaatggacgattttctgaccgcccaccacgagatgggccacatccagtatgatatggcatacgcagcacagcccttcctgctgaggaatggagccaacgagggctttcacgaggccgtgggcgagatcatgtctctgagcgccgccacaccaaagcacctgaagtccatcggcctgctgtctcccgacttccaggaggataacgagaccgagatcaattttctgctgaagcaggccctgaccatcgtgggcacactgcccttcacctatatgctggagaagtggagatggatggtgtttaagggcgagatccctaaggaccagtggatgaagaagtggtgggagatgaagagggagatcgtgggagtggtggagcccgtgcctcacgacgagacctactgtgatcccgcctccctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgcgcctgggcaagagcgagccatggaccctggccctggagaacgtggtgggagccaagaatatgaacgtgcggcccctgctgaattatttcgagcctctgtttacatggctgaaggatcagaataagaacagctttgtgggctggtctaccgactggagcccctacgccgatcagtccatcaaggtgagaatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatcagctttaacttctttgtgacagccccaaagaacgtgagcgatatcatccccagaaccgaggtggagaaggccatccggatgagccggtccagaatcaacgacgccttccgcctgaatgataactccctggagtttctgggcatccagcctaccctgggcccacctaatcagccacccgtgagcgagccaaagagctccgacaagacccacacatgccctccatgtccggcgccagaggccgccggaggacctagcgtgttcctgtttccccctaagccaaaggatacactgatgatcagccggacaccagaggtgacctgcgtggtggtgtctgtgagccacgaggaccccgaggtgaagttcaactggtatgtggatggcgtggaggtgcacaatgccaagaccaagcctagagaggagcagtataacagcacatacagggtggtgtccgtgctgaccgtgctgcaccaggactggctgaacggcaaggagtacaagtgcaaggtgtctaataaggccctgcctgccccaatcgagaagaccatcagcaaggcaaagggacagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 22 - protein, 954 aa, synthetic construct
  source 1..954: note = Chain H2 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 23 - DNA, 2862 bp, synthetic construct
  source 1..2862: mol_type = other DNA; note = Chain H2 variant
cagtccacaatcgaggagcaggccaagaccttcctggacaagtttaaccacgaggccgaggatctgttctatcagagctccctggcctcttggaactacaataccaacatcacagaggagaatgtgcagaacatgaacaatgccggggacaagtggagcgcctttctgaaggagcagtccacactggcccagatgtaccctctgcaggagatccagaacctgaccgtgaagctgcagctgcaggccctgcagcagaatggctctagcgtgctgtctgaggataagagcaagaggctgaatacaatcctgaacaccatgtccacaatctattctaccggcaaggtgtgcaatccagacaacccccaggagtgtctgctgctggagcctggcctgaatgagatcatggccaacagcctggattacaatgagaggctgtgggcatgggagtcttggcgcagcgaagtgggcaagcagctgcggccactgtacgaggagtatgtggtgctgaagaacgagatggccagagccaatcactacgaggactatggcgattactggaggggcgactacgaggtgaacggcgtggacggctacgattatagcagaggccagctgatcgaggatgtggagcacacattcgaggagatcaagcccctgtatgagcacctgcacgcctacgtgagggccaagctgatgaacgcctatccttcctacatctctccaatcggatgcctgcccgcccacctgctgggcgacatgtggggccgcttctggacaaacctgtactccctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccctggcaacgtgcagaaggccgtgtgccacccaacagcctgggacctgggcaagggcgatttcagaatcctgatgtgcaccaaggtgacaatggacgattttctgaccgcccacaatgagatgggcaacatccagtatgatatggcatacgcagcacagcccttcctgctgaggaatggagccaacgagggctttcacgaggccgtgggcgagatcatgtctctgagcgccgccacacccaagcacctgaagtccatcggcctgctgtctcctgacttccaggaggataacgagaccgagatcaattttctgctgaagcaggccctgaccatcgtgggcacactgcccttcacctatatgctggagaagtggagatggatggtgtttaagggcgagatccctaaggaccagtggatgaagaagtggtgggagatgaagagggagatcgtgggagtggtggagcccgtgcctcacgacgagacctactgtgatcccgccagcctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgcgcctgggcaagtccgagccatggaccctggccctggagaacgtggtgggagccaagaatatgaacgtgcggcccctgctgaattatttcgagcctctgtttacatggctgaaggatcagaataagaacagctttgtgggctggtctaccgactggagcccctacgccgatcagtccatcaaggtgcgcatcagcctgaagtccgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatctcttttaacttctttgtgacagccccaaagaatgtgagcgatatcatccccagaaccgaggtggagaaggccatccggatgagccggtccagaatcaacgacgccttccggctgaatgataactctctggagtttctgggcatccagccaaccctgggcccacctaatcagccacccgtgagcgagcccaagagctccgacaagacccacacatgccctccatgtccggcgccagaggccgccggaggaccaagcgtgttcctgtttccacccaagcccaaggacaccctgatgatctcccggaccccagaggtgacatgcgtggtggtgagcgtgtcccacgaggaccccgaggtgaagtttaactggtacgtggatggcgtggaggtgcacaatgccaagacaaagccccgggaggagcagtacaattctacctatagagtggtgagcgtgctgacagtgctgcaccaggattggctgaacggcaaggagtataagtgtaaggtgagcaataaggccctgccagcccccatcgagaagaccatctccaaggccaagggccagcctcgcgaaccacaggtctacgtgctgccccctagccgcgacgaactgactaaaaatcaggtctctctgctgtgtctggtcaaaggattctacccttccgacatcgccgtggagtgggaaagtaacggccagcccgagaacaattacctgacctggccccctgtgctggactctgatgggagtttctttctgtattcaaagctgacagtcgataaaagccggtggcagcagggcaatgtgttcagctgctccgtcatgcacgaagcactgcacaaccattacactcagaagtccctgtccctgtcacctggc

SEQ ID NO: 24 - protein, 961 aa, synthetic construct
  source 1..961: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSVPPPPPCCHPRLSLHRPALEDLLLGSEANLTCTLTGLRDASGVTFTWTPSSGKSAVQGPPERDLCGCYSVSSVLPGSAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 25 - DNA, 2883 bp, synthetic construct
  source 1..2883: mol_type = other DNA; note = Chain H1 variant
cagtctacaatcgaggagcaggccaagaccttcctggacaagtttaaccacgaggccgaggatctgttctatcagagctccctggcctcctggaactacaataccaacatcacagaggagaatgtgcagaacatgaacaatgccggggacaagtggtccgcctttctgaaggagcagtctacactggcccagatgtaccccctgcaggagatccagaacctgaccgtgaagctgcagctgcaggccctgcagcagaatggctctagcgtgctgagcgaggataagtccaagaggctgaatacaatcctgaacaccatgtctacaatctatagcaccggcaaggtgtgcaatccagacaacccccaggagtgtctgctgctggagccaggcctgaatgagatcatggccaacagcctggattacaatgagaggctgtgggcatgggagagctggcgctccgaagtgggcaagcagctgagacccctgtacgaggagtatgtggtgctgaagaacgagatggccagagccaatcactacgaggactatggcgattactggaggggcgactacgaggtgaacggcgtggacggctacgattatagcagaggccagctgatcgaggatgtggagcacacattcgaggagatcaagcccctgtatgagcacctgcacgcctacgtgagggccaagctgatgaacgcctatccttcttacatcagcccaatcggatgcctgcccgcccacctgctgggcgacatgtggggccgcttctggacaaacctgtactccctgaccgtgccttttggccagaagccaaatatcgacgtgaccgatgccatggtggaccaggcctgggatgcccagcggatcttcaaggaggccgagaagttcttcgtgagcgtgggcctgcccaacatgacacagggcttttgggagaatagcatgctgaccgaccctggcaacgtgcagaaggccgtgtgccacccaacagcctgggacctgggcaagggcgatttcagaatcctgatgtgcaccaaggtgacaatggacgattttctgaccgcccaccacgagatgggccacatccagtatgatatggcctacgccgcccagcctttcctgctgaggaatggagccaacgagggctttcacgaggccgtgggcgagatcatgagcctgtccgccgccacacccaagcacctgaagtctatcggcctgctgagccctgacttccaggaggataacgagaccgagatcaattttctgctgaagcaggccctgaccatcgtgggcacactgcctttcacctatatgctggagaagtggagatggatggtgtttaagggcgagatcccaaaggaccagtggatgaagaagtggtgggagatgaagagggagatcgtgggagtggtggagcccgtgcctcacgacgagacctactgtgatccagccagcctgttccacgtgtccaacgactattcttttatcaggtactatacccgcacactgtaccagttccagtttcaggaggccctgtgccaggcagcaaagcacgagggaccactgcacaagtgtgatatctctaacagcacagaggccggacagaagctgttcaatatgctgcgcctgggcaagtccgagccttggaccctggccctggagaacgtggtgggagccaagaatatgaacgtgcggcccctgctgaattatttcgagcctctgtttacatggctgaaggatcagaataagaactcctttgtgggctggagcaccgactggtccccttacgccgatcagtctatcaaggtgcgcatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatcagctttaacttctttgtgacagccccaaagaacgtgagcgatatcatccccagaaccgaggtggagaaggccatccggatgtcccggtctagaatcaacgacgccttccggctgaatgataacagcctggagtttctgggcatccagccaaccctgggcccacctaatcagccacccgtgtccgtgcctccaccacctccatgctgtcaccctaggctgtccctgcacagaccagccctggaggacctgctgctgggctctgaggccaatctgacatgtaccctgaccggcctgagggatgccagcggagtgaccttcacatggaccccatctagcggcaagtccgccgtgcagggacctccagagagggatctgtgcggatgttactccgtgtcctctgtgctgccaggctctgccgagccttggaaccacggcaagacattcacctgcacagccgcctatccagagtccaagacacccctgaccgccacactgtctaagagcggcaacacctttcggccggaggtgcacctgctgccacctccatctgaggagctggccctgaatgagctggtgaccctgacatgtctggcccggggcttctctcctaaggatgtgctggtgagatggctgcagggcagccaggagctgccaagggagaagtacctgacctgggcctctcgccaggagccaagccagggaaccacaacctttgccgtgacctctatcctgcgggtggccgccgaggactggaagaagggcgataccttcagctgcatggtgggacacgaggccctgcctctggcctttacccagaagacaatcgaccggttagccgggaagccaacacacgtgaatgtgagcgtggtcatggccgaggtggatggcacctgttat

SEQ ID NO: 26 - protein, 961 aa, synthetic construct
  source 1..961: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSVPPPPPCCHPRLSLHRPALEDLLLGSEANLTCTLTGLRDASGVTFTWTPSSGKSAVQGPPERDLCGCYSVSSVLPGSAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 27 - DNA, 2883 bp, synthetic construct
  source 1..2883: mol_type = other DNA; note = Chain H1 variant
cagtctacaatcgaggagcaggccaagaccttcctggacaagtttaaccacgaggccgaggatctgttctatcagagctccctggcctcctggaactacaataccaacatcacagaggagaatgtgcagaacatgaacaatgccggggacaagtggtccgcctttctgaaggagcagtctacactggcccagatgtaccccctgcaggagatccagaacctgaccgtgaagctgcagctgcaggccctgcagcagaatggctctagcgtgctgagcgaggataagtccaagaggctgaatacaatcctgaacaccatgtctacaatctatagcaccggcaaggtgtgcaatccagacaacccccaggagtgtctgctgctggagccaggcctgaatgagatcatggccaacagcctggattacaatgagaggctgtgggcatgggagagctggcgctccgaagtgggcaagcagctgagacccctgtacgaggagtatgtggtgctgaagaacgagatggccagagccaatcactacgaggactatggcgattactggaggggcgactacgaggtgaacggcgtggacggctacgattatagcagaggccagctgatcgaggatgtggagcacacattcgaggagatcaagcccctgtatgagcacctgcacgcctacgtgagggccaagctgatgaacgcctatccttcttacatcagcccaatcggatgcctgcccgcccacctgctgggcgacatgtggggccgcttctggacaaacctgtactccctgaccgtgccttttggccagaagccaaatatcgacgtgaccgatgccatggtggaccaggcctgggatgcccagcggatcttcaaggaggccgagaagttcttcgtgagcgtgggcctgcccaacatgacacagggcttttgggagaatagcatgctgaccgaccctggcaacgtgcagaaggccgtgtgccacccaacagcctgggacctgggcaagggcgatttcagaatcctgatgtgcaccaaggtgacaatggacgattttctgaccgcccacaacgagatgggcaacatccagtatgatatggcctacgccgcccagcctttcctgctgaggaatggagccaacgagggctttcacgaggccgtgggcgagatcatgagcctgtccgccgccacacccaagcacctgaagtctatcggcctgctgagccctgacttccaggaggataacgagaccgagatcaattttctgctgaagcaggccctgaccatcgtgggcacactgcctttcacctatatgctggagaagtggagatggatggtgtttaagggcgagatcccaaaggaccagtggatgaagaagtggtgggagatgaagagggagatcgtgggagtggtggagcccgtgcctcacgacgagacctactgtgatccagccagcctgttccacgtgtccaacgactattcttttatcaggtactatacccgcacactgtaccagttccagtttcaggaggccctgtgccaggcagcaaagcacgagggaccactgcacaagtgtgatatctctaacagcacagaggccggacagaagctgttcaatatgctgcgcctgggcaagtccgagccttggaccctggccctggagaacgtggtgggagccaagaatatgaacgtgcggcccctgctgaattatttcgagcctctgtttacatggctgaaggatcagaataagaactcctttgtgggctggagcaccgactggtccccttacgccgatcagtctatcaaggtgcgcatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatcagctttaacttctttgtgacagccccaaagaacgtgagcgatatcatccccagaaccgaggtggagaaggccatccggatgtcccggtctagaatcaacgacgccttccggctgaatgataacagcctggagtttctgggcatccagccaaccctgggcccacctaatcagccacccgtgtccgtgcctccaccacctccatgctgtcaccctaggctgtccctgcacagaccagccctggaggacctgctgctgggctctgaggccaatctgacatgtaccctgaccggcctgagggatgccagcggagtgaccttcacatggaccccatctagcggcaagtccgccgtgcagggacctccagagagggatctgtgcggatgttactccgtgtcctctgtgctgccaggctctgccgagccttggaaccacggcaagacattcacctgcacagccgcctatccagagtccaagacacccctgaccgccacactgtctaagagcggcaacacctttcggccggaggtgcacctgctgccacctccatctgaggagctggccctgaatgagctggtgaccctgacatgtctggcccggggcttctctcctaaggatgtgctggtgagatggctgcagggcagccaggagctgccaagggagaagtacctgacctgggcctctcgccaggagccaagccagggaaccacaacctttgccgtgacctctatcctgcgggtggccgccgaggactggaagaagggcgataccttcagctgcatggtgggacacgaggccctgcctctggcctttacccagaagacaatcgaccggttagccgggaagccaacacacgtgaatgtgagcgtggtcatggccgaggtggatggcacctgttat

SEQ ID NO: 28 - protein, 1072 aa, synthetic construct
  source 1..1072: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 29 - DNA, 3216 bp, synthetic construct
  source 1..3216: mol_type = other DNA; note = Chain H1 variant
cagtctacaatcgaggagcaggccaagaccttcctggacaagtttaaccacgaggccgaggatctgttctatcagagctccctggccagctggaactacaataccaacatcacagaggagaatgtgcagaacatgaacaatgccggggacaagtggtccgcctttctgaaggagcagtctacactggcccagatgtaccctctgcaggagatccagaacctgaccgtgaagctgcagctgcaggccctgcagcagaatggctctagcgtgctgagcgaggataagtccaagaggctgaatacaatcctgaacaccatgtctacaatctatagcaccggcaaggtgtgcaatccagacaacccccaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactacgaggtgaacggcgtggacggctacgattatagcagaggccagctgatcgaggatgtggagcacacattcgaggagatcaagcccctgtatgagcacctgcacgcctacgtgagggccaagctgatgaacgcctatccttcttacatcagcccaatcggatgcctgcccgcccacctgctgggcgacatgtggggccgcttctggacaaacctgtactccctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccctggcaacgtgcagaaggccgtgtgccacccaacagcctgggacctgggcaagggcgatttcagaatcctgatgtgcaccaaggtgacaatggacgattttctgaccgcccaccacgagatgggccacatccagtatgatatggcatacgcagcacagcccttcctgctgaggaatggagccaacgagggctttcacgaggccgtgggcgagatcatgagcctgtccgccgccacacccaagcacctgaagtctatcggcctgctgagccctgacttccaggaggataacgagaccgagatcaattttctgctgaagcaggccctgaccatcgtgggcacactgcctttcacctatatgctggagaagtggagatggatggtgtttaagggcgagatcccaaaggaccagtggatgaagaagtggtgggagatgaagagggagatcgtgggagtggtggagcccgtgcctcacgacgagacctactgtgatcccgcctccctgttccacgtgtccaacgactattcttttatcaggtactatacccgcacactgtaccagttccagtttcaggaggccctgtgccaggcagcaaagcacgagggcccactgcacaagtgtgatatctctaacagcacagaggccggacagaagctgttcaatatgctgcgcctgggcaagtctgagccctggaccctggccctggagaacgtggtgggagccaagaatatgaacgtgcggcccctgctgaattatttcgagcctctgtttacatggctgaaggatcagaataagaacagctttgtgggatggagcaccgactggtccccatacgccgatcagtccatcaaggtgcgcatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatctcttttaacttctttgtgacagccccaaagaatgtgagcgatatcatccccagaaccgaggtggagaaggccatccggatgtcccggtctagaatcaacgacgccttccggctgaatgataactccctggagtttctgggcatccagcccaccctgggcccacctaatcagccacccgtgagcgtgatcgcagagctgcctccaaaggtgtccgtgttcgtgcctcctagggacggcttctttggcaatccacgcaagagcaagctgatctgtcaggccaccggcttttctcccagacagatccaggtgagctggctgagagagggcaagcaagtgggctccggagtgaccacagatcaggtgcaggcagaggcaaaggagtctggaccaaccacatacaaggtgacctccacactgaccatcaaggagtctgactggctgagccagtccatgttcacctgccgggtggatcacagaggcctgacatttcagcagaacgccagctccatgtgcgtgcctgaccaggataccgccatcagagtgttcgcgatccctccatccttcgcctctatctttctgacaaagtccaccaagctgacatgcctggtgaccgacctgaccacatatgattccgtgaccatctcttggacaaggcagaacggcgaggccgtgaagacacacaccaacatctctgagagccaccccaatgccaccttcagcgccgtgggagaggcatccatctgcgaggacgattggaatagcggcgagcggtttacatgtaccgtgacacacaccgacctgcctagcccactgaagcagaccatctccaggcctaagggagtggccctgcaccggccggacgtgtacctgctgccccctgccagggagcagctgaacctgagagagagcgccacaatcacctgtctggtgaccggcttctcccctgccgacgtgtttgtgcagtggatgcagaggggacagcccctgtctcctgagaagtacgtgacaagcgcccccatgcctgagccacaggccccaggccggtatttcgcccactctatcctgaccgtgagcgaggaggagtggaacacaggcgagacatatacctgcgtggtggcacacgaggccctgcctaatagggtgaccgagcgcacagtggacaagagcaccggtaagccaacactgtacaatgtgagcctggtcatgtccgataccgccgggacctgttat

SEQ ID NO: 30 - protein, 1072 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 31 - DNA, 3216 bp, synthetic construct
  source 1..3216: mol_type = other DNA; note = Chain H1 variant
cagtctacaatcgaggagcaggccaagaccttcctggacaagtttaaccacgaggccgaggatctgttctatcagagctccctggccagctggaactacaataccaacatcacagaggagaatgtgcagaacatgaacaatgccggggacaagtggtccgcctttctgaaggagcagtctacactggcccagatgtaccctctgcaggagatccagaacctgaccgtgaagctgcagctgcaggccctgcagcagaatggctctagcgtgctgagcgaggataagtccaagaggctgaatacaatcctgaacaccatgtctacaatctatagcaccggcaaggtgtgcaatccagacaacccccaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactacgaggtgaacggcgtggacggctacgattatagcagaggccagctgatcgaggatgtggagcacacattcgaggagatcaagcccctgtatgagcacctgcacgcctacgtgagggccaagctgatgaacgcctatccttcttacatcagcccaatcggatgcctgcccgcccacctgctgggcgacatgtggggccgcttctggacaaacctgtactccctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccctggcaacgtgcagaaggccgtgtgccacccaacagcctgggacctgggcaagggcgatttcagaatcctgatgtgcaccaaggtgacaatggacgattttctgaccgcccacaacgagatgggcaacatccagtatgatatggcatacgcagcacagcccttcctgctgaggaatggagccaacgagggctttcacgaggccgtgggcgagatcatgagcctgtccgccgccacacccaagcacctgaagtctatcggcctgctgagccctgacttccaggaggataacgagaccgagatcaattttctgctgaagcaggccctgaccatcgtgggcacactgcctttcacctatatgctggagaagtggagatggatggtgtttaagggcgagatcccaaaggaccagtggatgaagaagtggtgggagatgaagagggagatcgtgggagtggtggagcccgtgcctcacgacgagacctactgtgatcccgcctccctgttccacgtgtccaacgactattcttttatcaggtactatacccgcacactgtaccagttccagtttcaggaggccctgtgccaggcagcaaagcacgagggcccactgcacaagtgtgatatctctaacagcacagaggccggacagaagctgttcaatatgctgcgcctgggcaagtctgagccctggaccctggccctggagaacgtggtgggagccaagaatatgaacgtgcggcccctgctgaattatttcgagcctctgtttacatggctgaaggatcagaataagaacagctttgtgggatggagcaccgactggtccccatacgccgatcagtccatcaaggtgcgcatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatctcttttaacttctttgtgacagccccaaagaatgtgagcgatatcatccccagaaccgaggtggagaaggccatccggatgtcccggtctagaatcaacgacgccttccggctgaatgataactccctggagtttctgggcatccagcccaccctgggcccacctaatcagccacccgtgagcgtgatcgcagagctgcctccaaaggtgtccgtgttcgtgcctcctagggacggcttctttggcaatccacgcaagagcaagctgatctgtcaggccaccggcttttctcccagacagatccaggtgagctggctgagagagggcaagcaagtgggctccggagtgaccacagatcaggtgcaggcagaggcaaaggagtctggaccaaccacatacaaggtgacctccacactgaccatcaaggagtctgactggctgagccagtccatgttcacctgccgggtggatcacagaggcctgacatttcagcagaacgccagctccatgtgcgtgcctgaccaggataccgccatcagagtgttcgcgatccctccatccttcgcctctatctttctgacaaagtccaccaagctgacatgcctggtgaccgacctgaccacatatgattccgtgaccatctcttggacaaggcagaacggcgaggccgtgaagacacacaccaacatctctgagagccaccccaatgccaccttcagcgccgtgggagaggcatccatctgcgaggacgattggaatagcggcgagcggtttacatgtaccgtgacacacaccgacctgcctagcccactgaagcagaccatctccaggcctaagggagtggccctgcaccggccggacgtgtacctgctgccccctgccagggagcagctgaacctgagagagagcgccacaatcacctgtctggtgaccggcttctcccctgccgacgtgtttgtgcagtggatgcagaggggacagcccctgtctcctgagaagtacgtgacaagcgcccccatgcctgagccacaggccccaggccggtatttcgcccactctatcctgaccgtgagcgaggaggagtggaacacaggcgagacatatacctgcgtggtggcacacgaggccctgcctaatagggtgaccgagcgcacagtggacaagagcaccggtaagccaacactgtacaatgtgagcctggtcatgtccgataccgccgggacctgttat

SEQ ID NO: 32 - protein, 231 aa, synthetic construct
  source 1..231: note = Chain H2 variant
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 33 - DNA, 693 bp, synthetic construct
  source 1..693: mol_type = other DNA; note = Chain H2 variant
gagccaaagagctccgacaagacccacacatgccccccttgtccggcgccagaggctgcaggaggaccaagcgtgttcctgtttccacccaagcctaaagacacactgatgatttcccgaacccccgaagtcacatgcgtggtcgtgtctgtgagtcacgaggaccctgaagtcaagttcaactggtacgtggatggcgtcgaggtgcataatgccaagactaaacctagggaggaacagtacaactcaacctatcgcgtcgtgagcgtcctgacagtgctgcaccaggattggctgaacggcaaagaatataagtgcaaagtgagcaataaggccctgcccgctcctatcgagaaaaccatttccaaggctaaagggcagcctcgcgaaccacaggtctacgtgtatcctccaagccgggacgagctgacaaagaaccaggtctccctgacttgtctggtgaaagggttttaccctagtgatatcgctgtggagtgggaatcaaatggacagccagagaacaattataagactaccccccctgtgctggacagtgatgggtcattcgcactggtctccaagctgacagtggacaaatctcggtggcagcagggaaatgtcttttcatgtagcgtgatgcatgaagcactgcacaaccattacacccagaagtcactgtcactgtcaccagga

SEQ ID NO: 34 - protein, 954 aa, synthetic construct
  source 1..954: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - DNA, 2862 bp, synthetic construct
  source 1..2862: mol_type = other DNA; note = Chain H1 variant
cagtccacaatcgaggagcaggccaagaccttcctggacaagtttaaccacgaggccgaggatctgttctatcagagctccctggccagctggaactacaataccaacatcacagaggagaatgtgcagaacatgaacaatgccggggacaagtggtccgcctttctgaaggagcagtctacactggcccagatgtaccctctgcaggagatccagaacctgaccgtgaagctgcagctgcaggccctgcagcagaatggctctagcgtgctgtctgaggataagagcaagcggctgaatacaatcctgaacaccatgtccacaatctattctaccggcaaggtgtgcaatcccgacaaccctcaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactacgaggtgaacggcgtggacggctacgattattccagaggccagctgatcgaggatgtggagcacacattcgaggagatcaagccactgtatgagcacctgcacgcctacgtgagggccaagctgatgaacgcctatccttcttacatcagcccaatcggatgcctgcccgcccacctgctgggcgacatgtggggccgcttctggacaaacctgtacagcctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccccggcaacgtgcagaaggccgtgtgccaccctacagcctgggacctgggcaagggcgatttcagaatcctgatgtgcaccaaggtgacaatggacgattttctgaccgcccaccacgagatgggccacatccagtatgatatggcatacgcagcacagcccttcctgctgaggaatggagccaacgagggctttcacgaggccgtgggcgagatcatgtctctgagcgccgccacaccaaagcacctgaagtccatcggcctgctgtctcccgacttccaggaggataacgagaccgagatcaattttctgctgaagcaggccctgaccatcgtgggcacactgcccttcacctatatgctggagaagtggagatggatggtgtttaagggcgagatccctaaggaccagtggatgaagaagtggtgggagatgaagagggagatcgtgggagtggtggagcccgtgcctcacgacgagacctactgtgatcccgcctccctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgcgcctgggcaagagcgagccatggaccctggccctggagaacgtggtgggagccaagaatatgaacgtgcggcccctgctgaattatttcgagcctctgtttacatggctgaaggatcagaataagaacagctttgtgggctggtctaccgactggagcccctacgccgatcagtccatcaaggtgagaatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatcagctttaacttctttgtgacagccccaaagaacgtgagcgatatcatccccagaaccgaggtggagaaggccatccggatgagccggtccagaatcaacgacgccttccgcctgaatgataactccctggagtttctgggcatccagcctaccctgggcccacctaatcagccacccgtgagcgagccaaagagctccgacaagacccacacatgccctccatgtccggcgccagaggccgccggaggacctagcgtgttcctgtttccccctaagccaaaggatacactgatgatcagccggacaccagaggtgacctgcgtggtggtgtctgtgagccacgaggaccccgaggtgaagttcaactggtatgtggatggcgtggaggtgcacaatgccaagaccaagcctagagaggagcagtataacagcacatacagggtggtgtccgtgctgaccgtgctgcaccaggactggctgaacggcaaggagtacaagtgcaaggtgtctaataaggccctgcctgccccaatcgagaagaccatcagcaaggcaaagggacagcctcgcgaaccacaggtgtacactctgcctccatctcgggacgagctgactaagaaccaggtcagtctgacctgtctggtgaaaggattctatcccagcgatatcgctgtggagtgggaatccaatggccagcctgagaacaattacaagaccacaccccctgtgctggactctgatggcagtttctttctgtatagtaagctgaccgtcgataaatcacgatggcagcaggggaacgtgttcagctgttcagtgatgcacgaagccctgcacaaccattacacccagaagagcctgagcctgtctcccggc

SEQ ID NO: 36 - protein, 954 aa, synthetic construct
  source 1..954: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - DNA, 2862 bp, synthetic construct
  source 1..2862: mol_type = other DNA; note = Chain H1 variant 27620
cagtccacaatcgaggagcaggccaagaccttcctggacaagtttaaccacgaggccgaggatctgttctatcagagctccctggcctcttggaactacaataccaacatcacagaggagaatgtgcagaacatgaacaatgccggggacaagtggagcgcctttctgaaggagcagtccacactggcccagatgtaccctctgcaggagatccagaacctgaccgtgaagctgcagctgcaggccctgcagcagaatggctctagcgtgctgtctgaggataagagcaagaggctgaatacaatcctgaacaccatgtccacaatctattctaccggcaaggtgtgcaatccagacaacccccaggagtgtctgctgctggagcctggcctgaatgagatcatggccaacagcctggattacaatgagaggctgtgggcatgggagtcttggcgcagcgaagtgggcaagcagctgcggccactgtacgaggagtatgtggtgctgaagaacgagatggccagagccaatcactacgaggactatggcgattactggaggggcgactacgaggtgaacggcgtggacggctacgattatagcagaggccagctgatcgaggatgtggagcacacattcgaggagatcaagcccctgtatgagcacctgcacgcctacgtgagggccaagctgatgaacgcctatccttcctacatctctccaatcggatgcctgcccgcccacctgctgggcgacatgtggggccgcttctggacaaacctgtactccctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccctggcaacgtgcagaaggccgtgtgccacccaacagcctgggacctgggcaagggcgatttcagaatcctgatgtgcaccaaggtgacaatggacgattttctgaccgcccacaatgagatgggcaacatccagtatgatatggcatacgcagcacagcccttcctgctgaggaatggagccaacgagggctttcacgaggccgtgggcgagatcatgtctctgagcgccgccacacccaagcacctgaagtccatcggcctgctgtctcctgacttccaggaggataacgagaccgagatcaattttctgctgaagcaggccctgaccatcgtgggcacactgcccttcacctatatgctggagaagtggagatggatggtgtttaagggcgagatccctaaggaccagtggatgaagaagtggtgggagatgaagagggagatcgtgggagtggtggagcccgtgcctcacgacgagacctactgtgatcccgccagcctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgcgcctgggcaagtccgagccatggaccctggccctggagaacgtggtgggagccaagaatatgaacgtgcggcccctgctgaattatttcgagcctctgtttacatggctgaaggatcagaataagaacagctttgtgggctggtctaccgactggagcccctacgccgatcagtccatcaaggtgcgcatcagcctgaagtccgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatctcttttaacttctttgtgacagccccaaagaatgtgagcgatatcatccccagaaccgaggtggagaaggccatccggatgagccggtccagaatcaacgacgccttccggctgaatgataactctctggagtttctgggcatccagccaaccctgggcccacctaatcagccacccgtgagcgagcccaagagctccgacaagacccacacatgccctccatgtccggcgccagaggccgccggaggaccaagcgtgttcctgtttccacccaagcccaaggacaccctgatgatctcccggaccccagaggtgacatgcgtggtggtgagcgtgtcccacgaggaccccgaggtgaagtttaactggtacgtggatggcgtggaggtgcacaatgccaagacaaagccccgggaggagcagtacaattctacctatagagtggtgagcgtgctgacagtgctgcaccaggattggctgaacggcaaggagtataagtgtaaggtgagcaataaggccctgccagcccccatcgagaagaccatctccaaggccaagggccagcctcgcgaaccacaggtgtacactctgcctccatctcgggacgagctgactaagaaccaggtcagtctgacctgtctggtgaaaggattctatcccagcgatatcgctgtggagtgggaatccaatggccagcctgagaacaattacaagaccacaccccctgtgctggactctgatggcagtttctttctgtatagtaagctgaccgtcgataaatcacgatggcagcaggggaacgtgttcagctgttcagtgatgcacgaagccctgcacaaccattacacccagaagagcctgagcctgtctcccggc

SEQ ID NO: 38 - protein, 1692 aa, synthetic construct
  source 1..1692: note = Chain H1 variant
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 39 - DNA, 5076 bp, synthetic construct
  source 1..5076: mol_type = other DNA; note = Chain H1 variant
cagtccacaatcgaggagcaggccaagaccttcctggacaagtttaaccacgaggccgaggatctgttctatcagagctccctggcctcttggaactacaataccaacatcacagaggagaatgtgcagaacatgaacaatgccggggacaagtggagcgcctttctgaaggagcagtccacactggcccagatgtaccctctgcaggagatccagaacctgaccgtgaagctgcagctgcaggccctgcagcagaatggctctagcgtgctgtctgaggataagagcaagaggctgaatacaatcctgaacaccatgtccacaatctattctaccggcaaggtgtgcaatccagacaacccccaggagtgtctgctgctggagcctggcctgaatgagatcatggccaacagcctggattacaatgagaggctgtgggcatgggagtcttggcgcagcgaagtgggcaagcagctgcggccactgtacgaggagtatgtggtgctgaagaacgagatggccagagccaatcactacgaggactatggcgattactggaggggcgactacgaggtgaacggcgtggacggctacgattatagcagaggccagctgatcgaggatgtggagcacacattcgaggagatcaagcccctgtatgagcacctgcacgcctacgtgagggccaagctgatgaacgcctatccttcctacatctctccaatcggatgcctgcccgcccacctgctgggcgacatgtggggccgcttctggacaaacctgtactccctgaccgtgccttttggccagaagccaaatatcgacgtgaccgatgccatggtggaccaggcctgggatgcccagcggatcttcaaggaggccgagaagttcttcgtgagcgtgggcctgccaaacatgacacagggcttttgggagaacagcatgctgaccgaccctggcaacgtgcagaaggccgtgtgccacccaacagcctgggacctgggcaagggcgatttcagaatcctgatgtgcaccaaggtgacaatggacgattttctgaccgcccacaatgagatgggcaacatccagtatgatatggcatacgcagcacagcccttcctgctgaggaatggagccaacgagggctttcacgaggccgtgggcgagatcatgtctctgagcgccgccacacccaagcacctgaagtccatcggcctgctgtctcctgacttccaggaggataacgagaccgagatcaattttctgctgaagcaggccctgaccatcgtgggcacactgcccttcacctatatgctggagaagtggagatggatggtgtttaagggcgagatccctaaggaccagtggatgaagaagtggtgggagatgaagagggagatcgtgggagtggtggagcccgtgcctcacgacgagacctactgtgatcccgccagcctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgcgcctgggcaagtccgagccatggaccctggccctggagaacgtggtgggagccaagaatatgaacgtgcggcccctgctgaattatttcgagcctctgtttacatggctgaaggatcagaataagaacagctttgtgggctggtctaccgactggagcccctacgccgatcagtccatcaaggtgcgcatcagcctgaagtccgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcctagaatctcttttaacttctttgtgacagccccaaagaatgtgagcgatatcatccccagaaccgaggtggagaaggccatccggatgagccggtccagaatcaacgacgccttccggctgaatgataactctctggagtttctgggcatccagccaaccctgggcccacctaatcagccacccgtgagcgagcccaagagctccgacaagacccacacatgccctccatgtccggcgccagaggccgccggaggaccaagcgtgttcctgtttccacccaagcccaaggacaccctgatgatctcccggaccccagaggtgacatgcgtggtggtgagcgtgtcccacgaggaccccgaggtgaagtttaactggtacgtggatggcgtggaggtgcacaatgccaagacaaagccccgggaggagcagtacaattctacctatagagtggtgagcgtgctgacagtgctgcaccaggattggctgaacggcaaggagtataagtgtaaggtgagcaataaggccctgccagcccccatcgagaagaccatctccaaggccaagggccagcctcgcgagccccaggtgtataccctgccccctagcagagacgagctgacaaagaaccaggtgtccctgacctgcctggtgaagggcttctacccttccgatatcgccgtggagtgggagtctaatggccagccagagaacaattataagaccacaccacccgtgctggactccgatggctctttctttctgtacagcaagctgacagtggacaagtccaggtggcagcagggcaacgtgttcagctgttccgtgatgcacgaggccctgcacaatcactacacccagaagtctctgagcttaagccctggaggaggaggaggctctggaggaggaggcagcggcggaggaggctctcagagcacaatcgaggagcaggccaagaccttcctggacaagtttaaccacgaggccgaggatctgttctatcagagctccctggcctcctggaactacaataccaacatcacagaggagaatgtgcagaacatgaacaatgccggggacaagtggtctgcctttctgaaggagcagagcacactggcccagatgtacccactgcaggagatccagaacctgaccgtgaagctgcagctgcaggccctgcagcagaatggctctagcgtgctgagcgaggataagtccaagaggctgaatacaatcctgaacaccatgtctacaatctatagcaccggcaaggtgtgcaatcctgacaacccacaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactatgaggtgaacggcgtggacggctacgattatagccggggccagctgatcgaggatgtggagcacacattcgaggagatcaagcccctgtatgagcacctgcacgcctacgtgcgggccaagctgatgaacgcctatccctcttacatcagccctatcggatgcctgccagcccacctgctgggcgacatgtggggcagattctggacaaacctgtacagcctgaccgtgccctttggccagaagcctaatatcgacgtgaccgatgccatggtggaccaggcctgggatgcccagaggatcttcaaggaggccgagaagttcttcgtgagcgtgggcctgccaaacatgacacagggcttttgggagaacagcatgctgaccgaccccggcaacgtgcagaaggccgtgtgccaccctacagcctgggacctgggcaagggcgatttccgcatcctgatgtgcaccaaggtgacaatggacgattttctgaccgcccacaatgagatgggcaacatccagtatgatatggcatacgcagcacagcccttcctgctgaggaatggagccaacgagggctttcacgaggccgtgggcgagatcatgtccctgtctgccgccacaccaaagcacctgaagtccatcggcctgctgtctcccgacttccaggaggataacgagaccgagatcaattttctgctgaagcaggccctgaccatcgtgggcacactgcccttcacctatatgctggagaagtggcgctggatggtgtttaagggcgagatccctaaggaccagtggatgaagaagtggtgggagatgaagcgggagatcgtgggagtggtggagccagtgccccacgacgagacctactgtgatcccgcctccctgttccacgtgtccaacgactattcttttatcaggtactatacccgcacactgtaccagttccagtttcaggaggccctgtgccaggcagcaaagcacgagggcccactgcacaagtgtgatatcagcaactccacagaggccggacagaagctgttcaatatgctgagactgggcaagagcgagccatggaccctggccctggagaacgtggtgggagccaagaatatgaacgtgaggccactgctgaattatttcgagcccctgtttacatggctgaaggatcagaataagaacagctttgtgggctggagcaccgactggtccccttacgccgatcagtccatcaaggtgcgcatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcccagaatctcttttaacttctttgtgacagcccctaagaatgtgagcgatatcatcccacgcaccgaggtggagaaggccatccggatgtctcggagcagaatcaacgacgccttccggctgaatgataactctctggagtttctgggcatccagcctaccctgggccctccaaatcagccccctgtgagc

SEQ ID NO: 40 - protein, 969 aa, synthetic construct
  source 1..969: note = Chain H1 variant
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 41 - DNA, 2907 bp, synthetic construct
  source 1..2907: mol_type = other DNA; note = Chain H1 variant
gagccaaagagctccgacaagacccacacatgccccccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgagccccaggtgtataccctgccccctagcagagacgagctgacaaagaaccaggtgtccctgacctgcctggtgaagggcttctacccttccgatatcgccgtggagtgggagtctaatggccagccagagaacaattataagaccacaccacccgtgctggactccgatggctctttctttctgtacagcaagctgacagtggacaagtccaggtggcagcagggcaacgtgttcagctgttccgtgatgcacgaggccctgcacaatcactacacccagaagtctctgagcttaagccctggaggaggaggaggctctggaggaggaggcagcggcggaggaggctctcagagcacaatcgaggagcaggccaagaccttcctggacaagtttaaccacgaggccgaggatctgttctatcagagctccctggcctcctggaactacaataccaacatcacagaggagaatgtgcagaacatgaacaatgccggggacaagtggtctgcctttctgaaggagcagagcacactggcccagatgtacccactgcaggagatccagaacctgaccgtgaagctgcagctgcaggccctgcagcagaatggctctagcgtgctgagcgaggataagtccaagaggctgaatacaatcctgaacaccatgtctacaatctatagcaccggcaaggtgtgcaatcctgacaacccacaggagtgtctgctgctggagcctggcctgaatgagatcatggccaacagcctggattacaatgagaggctgtgggcatgggagagctggcgctccgaagtgggcaagcagctgcggccactgtacgaggagtatgtggtgctgaagaacgagatggccagagccaatcactacgaggactatggcgattactggaggggcgactatgaggtgaacggcgtggacggctacgattatagccggggccagctgatcgaggatgtggagcacacattcgaggagatcaagcccctgtatgagcacctgcacgcctacgtgcgggccaagctgatgaacgcctatccctcttacatcagccctatcggatgcctgccagcccacctgctgggcgacatgtggggcagattctggacaaacctgtacagcctgaccgtgccctttggccagaagcctaatatcgacgtgaccgatgccatggtggaccaggcctgggatgcccagaggatcttcaaggaggccgagaagttcttcgtgagcgtgggcctgccaaacatgacacagggcttttgggagaacagcatgctgaccgaccccggcaacgtgcagaaggccgtgtgccaccctacagcctgggacctgggcaagggcgatttccgcatcctgatgtgcaccaaggtgacaatggacgattttctgaccgcccacaatgagatgggcaacatccagtatgatatggcatacgcagcacagcccttcctgctgaggaatggagccaacgagggctttcacgaggccgtgggcgagatcatgtccctgtctgccgccacaccaaagcacctgaagtccatcggcctgctgtctcccgacttccaggaggataacgagaccgagatcaattttctgctgaagcaggccctgaccatcgtgggcacactgcccttcacctatatgctggagaagtggcgctggatggtgtttaagggcgagatccctaaggaccagtggatgaagaagtggtgggagatgaagcgggagatcgtgggagtggtggagccagtgccccacgacgagacctactgtgatcccgcctccctgttccacgtgtccaacgactattcttttatcaggtactatacccgcacactgtaccagttccagtttcaggaggccctgtgccaggcagcaaagcacgagggcccactgcacaagtgtgatatcagcaactccacagaggccggacagaagctgttcaatatgctgagactgggcaagagcgagccatggaccctggccctggagaacgtggtgggagccaagaatatgaacgtgaggccactgctgaattatttcgagcccctgtttacatggctgaaggatcagaataagaacagctttgtgggctggagcaccgactggtccccttacgccgatcagtccatcaaggtgcgcatctccctgaagtctgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcccagaatctcttttaacttctttgtgacagcccctaagaatgtgagcgatatcatcccacgcaccgaggtggagaaggccatccggatgtctcggagcagaatcaacgacgccttccggctgaatgataactctctggagtttctgggcatccagcctaccctgggccctccaaatcagccccctgtgagc

SEQ ID NO: 42 - protein, 969 aa, synthetic construct
  source 1..969: note = Chain H1 variant
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 43 - DNA, 2907 bp, synthetic construct
  source 1..2907: mol_type = other DNA; note = Chain H1 variant
gagccaaagagctccgacaagacccacacatgccccccttgtccggcgccagaggcagcaggaggaccaagcgtgttcctgtttccacccaagcccaaagacaccctgatgattagccgaacccctgaagtcacatgcgtggtcgtgtccgtgtctcacgaggacccagaagtcaagttcaactggtacgtggatggcgtcgaggtgcataatgccaagacaaaaccccgggaggaacagtacaacagcacctatagagtcgtgtccgtcctgacagtgctgcaccaggattggctgaacggcaaggaatataagtgcaaagtgtccaataaggccctgcccgctcctatcgagaaaaccatttctaaggcaaaaggccagcctcgcgagcctcaggtgtacgtgctgccaccttcccgcgacgagctgaccaagaatcaggtgtctctgctgtgcctggtgaagggcttctatccaagcgatatcgcagtggagtgggagtccaacggacagcccgagaacaattacctgacctggccacccgtgctggacagcgatggctccttctttctgtattccaagctgacagtggacaagtctagatggcagcagggcaacgtgttcagctgttccgtgatgcacgaggccctgcacaaccactacacccagaagtctctgagcttaagccctggaggaggaggaggctccggcggaggaggctctggaggaggaggcagccagtccacaatcgaggagcaggccaagaccttcctggacaagtttaaccacgaggccgaggatctgttctatcagagctccctggccagctggaactacaataccaacatcacagaggagaatgtgcagaacatgaacaatgccggggacaagtggtctgcctttctgaaggagcagagcacactggcccagatgtacccactgcaggagatccagaacctgaccgtgaagctgcagctgcaggccctgcagcagaatggctctagcgtgctgtctgaggataagagcaagcggctgaatacaatcctgaacaccatgtccacaatctattctaccggcaaggtgtgcaatcccgacaaccctcaggagtgtctgctgctggagcctggcctgaatgagatcatggccaactccctggattacaatgagaggctgtgggcatgggagtcttggcgcagcgaagtgggcaagcagctgcggccactgtacgaggagtatgtggtgctgaagaacgagatggccagagccaatcactacgaggactatggcgattactggaggggcgactacgaggtgaacggcgtggacggctacgattatagccggggccagctgatcgaggatgtggagcacacattcgaggagatcaagcccctgtatgagcacctgcacgcctacgtgcgggccaagctgatgaacgcctatccatcctacatctctccaatcggatgcctgcctgcccacctgctgggcgacatgtggggcagattctggacaaacctgtactccctgaccgtgccatttggccagaagcccaatatcgacgtgaccgatgccatggtggaccaggcctgggatgcccagaggatcttcaaggaggccgagaagttctttgtgagcgtgggcctgccaaacatgacacagggcttttgggagaacagcatgctgaccgacccaggcaacgtgcagaaggccgtgtgccacccaacagcatgggacctgggcaagggcgatttccgcatcctgatgtgcaccaaggtgacaatggacgattttctgaccgcccacaatgagatgggcaacatccagtatgatatggcatacgcagcacagcccttcctgctgcggaatggagccaacgagggctttcacgaggccgtgggcgagatcatgtctctgagcgccgccacacctaagcacctgaagagcatcggcctgctgtccccagacttccaggaggataacgagaccgagatcaattttctgctgaagcaggccctgaccatcgtgggcacactgcccttcacctatatgctggagaagtggcgctggatggtgtttaagggcgagatccctaaggaccagtggatgaagaagtggtgggagatgaagcgggagatcgtgggcgtggtggagcctgtgccacacgacgagacctactgtgatcccgccagcctgttccacgtgagcaacgactattcctttatcaggtactatacccgcacactgtaccagttccagtttcaggaggccctgtgccaggcagcaaagcacgagggcccactgcacaagtgtgatatctccaactctacagaggccggacagaagctgttcaatatgctgagactgggcaagtccgagccatggaccctggccctggagaacgtggtgggagccaagaatatgaacgtgaggcctctgctgaattatttcgagccactgtttacatggctgaaggatcagaataagaactctttcgtgggctggtctaccgactggagcccttacgccgatcagagcatcaaggtgagaatcagcctgaagtccgccctgggcgacaaggcctatgagtggaatgataacgagatgtacctgttcagatcctctgtggcctatgccatgaggcagtacttcctgaaggtgaagaaccagatgatcctgtttggcgaggaggacgtgcgggtggccaatctgaagcccagaatctcctttaacttctttgtgacagcccccaagaacgtgagcgatatcatccctcgcaccgaggtggagaaggccatccggatgagccggtccagaatcaacgacgccttcaggctgaatgataactccctggagtttctgggcatccagcctaccctgggcccacctaatcagccacccgtgtct

SEQ ID NO: 44 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGCNEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLCLENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 45 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTACNEMGNIQYDMAYAAQPFLLRNGANEGFHEAVGEIMSLSCATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 46 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQACKHEGPLHKCDISNSTECGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 47 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKECEKFFVSVGLPNMTQGFWENSMLTDPGNVQKAVCHPTAWDLGKGDFRILMCTKVTMDDFLTCHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 48 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDCQRIFKEAEKFFVSVGLPNMTQGFWENSMLTDPGNVQKAVCHPTAWDLGKGDFRILMCCKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 49 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYCMRQYFLKVKNQMILFGEEDVRVANLKPRISFNFFVTAPKNVSDIIPRTEVEKAIRMCRSRINDAFRLNDNSLEFLGIQPTLGPPNQPPVS

SEQ ID NO: 50 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDCTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGCLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 51 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTIYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 52 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFYKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 53 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKVVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 54 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIIGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 55 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDIEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 56 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFFEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 57 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFYEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 58 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKFLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 59 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCIKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 60 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFLYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 61 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSIAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 62 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVLNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 63 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLHAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 64 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILWCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 65 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILFCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 66 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYMFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 67 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLWEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 68 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLIAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 69 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFHEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 70 - skipped

SEQ ID NO: 71 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVIAPKNVSDIIPRTEVEKAIRMSRSRINDAFRLNDNSLEFLGIQPTLGPPNQPPVS

SEQ ID NO: 72 - protein, 723 aa, synthetic construct
  source 1..723: note = knock out and spike variant of Q18-S740
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSLAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

(19) United States
(12) Patent Application Publication
(10) Pub. No.: US 2024/0327815 A1
(43) Pub. Date: Oct. 3, 2024

(54) MULTIVALENT POLYPEPTIDE CONSTRUCTS CAPABLE OF BINDING VIRAL SPIKE PROTEINS AND METHODS OF USING THE SAME

(71) Applicant: Zymeworks BC Inc., Vancouver (CA)

(72) Inventors: Surjit Bhimarao Dixit, Vancouver (CA); Eric Escobar-Cabrera, Vancouver (CA); Florian Heinkel, Vancouver (CA); Meghan Marie Verstraete, Vancouver (CA)

(21) Appl. No.: 18/606,452

(22) Filed: Mar. 15, 2024

Related U.S. Application Data
(63) Continuation of application No. PCT/CA2022/051370, filed on Sep. 14, 2022.
(60) Provisional application No. 63/398,482, filed on Aug. 16, 2022.
(60) Provisional application No. 63/245,786, filed on Sep. 17, 2021.

(51) Int. Cl. C12N 9/48; A61K 38/00; A61P 31/14

(52) U.S. Cl. CPC C12N 9/485; A61P 31/14; C12Y 304/17023; A61K 38/00; C07K 2319/30

(57) ABSTRACT

The present disclosure describes multivalent (e.g., decavalent or dodecavalent) polypeptide constructs capable of binding to one or more viral spike protein(s) and capable of intercepting the interaction of a spike protein of a viral particle with a surface-bound ACE2 protein of a cell, pharmaceutical compositions comprising such constructs, and methods of producing and using the constructs for the treatment of, e.g., viral infections in a subject in need thereof.

### CROSS-REFERENCE

This application is a continuation of PCT/CA2022/051370, filed on Sep. 14, 2022, which claims the benefit of U.S. Provisional Application No. 63/245,786, filed Sep. 17, 2021, and U.S. Provisional Application No. 63/398,482, filed Aug. 16, 2022, which applications are incorporated herein by reference in their entirety for all purposes.

### SEQUENCE LISTING

The instant application contains a Sequence Listing which is hereby incorporated by reference in its entirety. Said XML copy, created on May 30, 2024, is named ZWI-079USWOC1_SL.xml, and is 157,023 bytes in size.

### TECHNICAL FIELD

The present disclosure generally relates to multivalent polypeptide constructs capable of binding viral spike protein(s), compositions (e.g., pharmaceutical compositions) comprising such constructs and methods of producing and using the constructs and related compositions for the treatment of a variety of viral diseases including those caused by coronaviruses, such as SARS-CoV and SARS-CoV-2.

### BACKGROUND

SARS-coronavirus 2 (SARS-CoV-2) infections are a public health emergency. Angiotensin Converting Enzyme 2 (ACE2) has been shown to be the cellular entry receptor for the SARS coronavirus 2 (SARS-CoV-2). Virus entry is mediated by interactions between the viral spike protein (trimeric complex of protein S, also known individually as protein S subunits (e.g., S1 and S2), and the ACE2 protein, an enzyme on a host cell membrane. The viral spike protein binds ACE2 via a receptor-binding domain (RBD) on the protein S1 subunit. See, e.g., Jing W, Procko E. ACE2-based decoy receptors for SARS coronavirus 2. Proteins. 2021; 1-14. Anti-spike protein antibodies have been developed and used to block ACE2 engagement and additional monoclonal antibody therapies are in clinical development. However, such approaches have only shown limited clinical success thus far and were shown to have decreased efficacy against newly emerging virus variants, such as delta and omicron (see, e.g., FDA revocation letter for bamlanivimab, Apr. 16, 2021).

Coronaviruses can have high mutation rates and new variants (e.g., the delta and omicron variants) with increased transmissibility and partial immune escape have emerged. Thus, there remains an urgent need for therapeutic modalities with improved anti-spike protein binding properties to limit or avoid the mutational escape by newly emerging virus variants.

### SUMMARY

One embodiment of the present disclosure relates to polypeptide constructs comprising an ACE2 protein and capable of binding a viral spike protein with the ACE2 protein. Hence, in some embodiments, the polypeptide constructs herein can be characterized as spike protein binding molecules. The polypeptide constructs described herein can comprise a soluble portion of the ACE2 protein, e.g., the portion responsible for spike protein binding and which is not anchored to the cell membrane (i.e., can be characterized as “soluble”). The polypeptide constructs described herein can be capable of intercepting and preventing the interaction between a spike protein associated with a viral particle and a surface-bound ACE2 protein of a target cell by competing for, and occupying the binding sites of, the viral spike protein using the ACE2 protein(s) of the construct. Hence, in various embodiments, an ACE2-containing polypeptide construct of this disclosure can prevent binding of the viral particle to the host or target cell via the ACE2 receptor by acting as a “decoy” for viral spike proteins through its ACE2 protein-containing binding domains. Thus, the polypeptide constructs of the present disclosure may also be referred to as ACE2 decoy polypeptide constructs.

In some embodiments, an ACE2 decoy polypeptide construct of the present disclosure can comprise an immunoglobin (Ig) Fc domain, or an Ig Fc scaffold comprising multiple (e.g., two or more) Fc domains, and at least one, two, three, five, ten or more binding units, wherein each binding unit can be coupled to an Fc polypeptide (e.g., IgG or an IgM Fc polypeptide) and comprises an angiotensin-converting enzyme 2 (ACE2) protein, or portion thereof, and wherein the ACE2 protein, or portion thereof, is capable of binding the receptor binding domain (RBD) of a spike protein of a virus. In various cases, the virus that can be targeted by an ACE2 decoy polypeptide construct of this disclosure can be a coronavirus, e.g., a severe acute respiratory syndrome (SARS) associated virus, such as SARS-CoV or SARS-CoV-2.

In some embodiments, the immunoglobulin Fc domain of an ACE2 decoy polypeptide construct described herein comprises an IgG Fe, IgA Fe, IgM Fc domain, or a combination thereof. In various embodiments, an ACE2 decoy polypeptide construct provided herein comprises one or more IgG Fc domain(s). Such one or more IgG Fc domain(s) can each be dimeric, e.g., homo- or heterodimeric, comprising a first and a second Fc polypeptide (see, e.g., FIGS. 1G-1H). In some embodiments, an ACE2 decoy polypeptide construct provided herein comprises one or more IgA Fc domain(s). Such one or more IgA Fc domain(s) can each be dimeric, e.g., homo- or heterodimeric, comprising a first and a second Fc polypeptide. In some cases, two or more dimeric IgA Fe domains form an IgA Fc scaffold in which the C-termini of the Fc polypeptides are coupled to one another, e.g., via a tailpiece assembly, a J-chain, or a combination thereof (see, e.g., FIGS. 1D-1F). In various other embodiments, an ACE2 decoy polypeptide construct provided herein comprises an IgM Fc scaffold, such as a pentameric IgM Fc scaffold comprising five dimeric Fe domains (see, e.g., FIG. 1A), or a hexameric IgM Fc scaffold comprising six dimeric Fe domains (see, e.g., FIG. 1B).

Hence, in some embodiments, an ACE2 decoy polypeptide construct described herein comprises a trimeric, tetrameric, pentameric or hexameric Fc scaffold. In some embodiments, an ACE2 decoy polypeptide construct provided herein comprises a pentameric or hexameric Fc scaffold. Such Fc scaffold be an IgM- or an IgM-derived Fc scaffold, or an IgA- or an IgA-derived scaffold. A scaffold that is derived from either IgA or IgM include those that either (i) comprise Fc polypeptides having amino acid sequence variation compared to IgA or IgM wildtype Fc polypeptides such as having about 80%, 90%, 95%, or 99% sequence identity compared to respective wildtype sequences, or (ii) contain additional molecules that can be coupled to the Fc scaffold, such as a binding domain comprising an ACE2 protein, or other atoms or molecules such as radioisotope(s), fluorophore(s), therapeutic molecule(s), etc.

In some embodiments, an ACE2 decoy polypeptide construct described herein that comprises a pentameric or a hexameric Fc scaffold (i.e., an Fc scaffold comprising either five or six dimeric Fe domains) can further comprise five to ten binding domains or six to twelve binding domains, respectively, wherein each binding domain comprises an ACE2 protein capable of binding a spike protein of a SARS-associated virus, and wherein each binding domain is coupled to an Fc polypeptide of the Fc scaffold such that each dimeric Fc domain of the pentameric or a hexameric Fc scaffold is coupled to at least one binding domain. In various cases, the ACE2 protein can comprise or consist of the extracellular (e.g., “soluble”) domain of a wildtype ACE2 protein such as a sequence set forth in SEQ ID NO: 2 or 3, or a modified variant thereof, as described herein.

Hence, one embodiment of the present disclosure relates to an ACE2 decoy polypeptide construct comprising a binding domain comprising an ACE2 extracellular domain, such binding domain comprising or consisting of amino acids Q18-S740 (SEQ ID NO: 2) or Q18-A614 (SEQ ID NO: 3) of the wild-type ACE2 extracellular domain protein with a sequence set forth in SEQ ID NO: 1.

Another embodiment of the present disclosure describes an ACE2 decoy polypeptide construct comprising a modified ACE2 extracellular domain protein binding unit comprising one or more amino acid substitutions relative to the sequence of amino acid residues Q18-S740 or Q18-A614 of SEQ ID NO: 1. In various aspects, such one or more amino acid substitutions increase the thermal stability of the modified ACE2 extracellular domain protein binding unit compared to an ACE2 extracellular domain protein binding unit that does not contain such mutations, e.g., a wildtype ACE2 domain. In various cases, such one or more amino acid substitutions are not located in the region of the ACE2 extracellular domain binding unit which contacts and interacts with the spike protein. As described herein, an increase in thermal stability of a modified binding domain of the present disclosure can be measured as an increase in melting temperature relative to an unmodified binding domain that lacks those one or more amino acid substitutions. Such increase in melting temperature can be about 1° C., 2° C., 3° C., 4° C., 5° C., 6° C., 7° C., or about 8° C., compared to a respective domain that does not comprise such amino acid modifications, and as measured, e.g., using differential scanning calorimetry (DSC). In some embodiments, such binding domain consists of an ACE2 protein having the sequence set forth in SEQ ID NO: 1, or a portion thereof, such as a sequence set forth in any one of SEQ ID Nos: 2-4.

In some embodiments, such one or more amino acid substitutions that can increase the stability (e.g., thermal stability) of an ACE2 protein and thereby enhance the developability of a polypeptide construct can be at any one or more of the positions 73, 180, 226, 293, 304, 311, 342, 360, 362, 372, 373, 396, 411, 412, 417, 423, 447, 453, 507, 520, 522, 526, 533, 550, 569, 647, 650, 686 and 707, based on the sequence set forth in SEQ ID NO: 1. In some embodiments, such one or more substitutions may introduce cysteine residue(s) to form additional disulfide bond(s). Such substitutions can include any one or more of A396C_596C, H373C_A412C, A533C_A550C, A311C_A372C, A304C_T362C_A650C_S707C and V293C_L423C. ACE2 proteins comprising such substitutions can comprise or consist of any one of the sequences set forth in SEQ ID NOs: 44-50 herein. In other embodiments, a modified ACE2 protein can have an increased stability by containing one or more amino acid substitutions that increase non-covalent interactions between certain segments of the ACE2 protein. Such substitutions can be selected from any one or more of L520I, L73Y, A342V, V447I, V226I, Q526F, Q526Y, H417F, T362I, T453L, V647I, S507L, S411H, M360W, M360F, Q522M, Y180W, S411I, Q526H, T686I and V647L. ACE2 proteins comprising such substitutions can comprise or consist of any one of the sequences set forth in SEQ ID NOs: 51-72, also referred to herein as stability “S” variants.

Another embodiment of the present disclosure relates to an ACE2 decoy polypeptide construct comprising an ACE2 extracellular domain binding unit comprising an amino acid sequence that is at least about 80%, 90%, 95%, 97% or at least about 99% identical to the ACE2 extracellular domain binding unit having the amino acid sequence set forth in any one of SEQ ID NOs: 1-4. In various embodiments, however, at least the region of the ACE2 protein that interacts with a spike protein is wildtype, i.e., is not modified using amino acid or other modifications. In some aspects, using such wildtype ACE2 sequence for engaging a viral spike protein in vitro or in vivo may reduce the probability of emerging escape viral mutants compared to instances in which a modified ACE2 sequence is used to intercept viral spike proteins, as further described herein.

Another embodiment of the present disclosure relates to an ACE2 decoy polypeptide construct comprising a pentameric or a hexameric Fc scaffold, wherein such scaffold is an IgM scaffold. In some embodiments, such ACE2 decoy polypeptide construct comprises a pentameric or hexameric IgM Fc scaffold comprising five or six dimeric Fc domains coupled to ten or twelve binding domains, respectively, wherein each binding domain comprises an ACE2 protein and is capable of binding the spike protein of a SARS-associated viruses, and wherein each of such binding domains is coupled either directly or via a first linker to an N-terminus of a first or second Fc polypeptide of one of the dimeric IgM Fc domains.

In some of such embodiment of the present disclosure, an ACE2 decoy polypeptide construct can further comprise a tailpiece assembly, wherein each first and second Fc polypeptide of the dimeric Fc domains comprises a C-terminal tailpiece sequence that, when bound together following expression of the FC polypeptide chains, e.g., through covalent (e.g., disulfide bonds) and/or non-covalent interactions, form the tailpiece assembly. In some embodiments, e.g., when the polypeptide construct comprises a pentameric IgM scaffold, each Fc polypeptide can comprise a tailpiece sequence having at least about 90%, 95% or 100% sequence identity to the sequence set forth in SEQ ID NO: 9. In various embodiments, such polypeptide construct further comprises a J-chain. Such J-chain can comprise or consist of an amino acid sequence having at least about 90%, 95%, 97%, 99% or 100% sequence identity to the amino acid sequence set forth in SEQ ID NO: 10 or SEQ ID NO: 12. In some embodiments, a J-chain can also be coupled to a binding domain comprising an ACE2 protein as described herein. Such an embodiment is illustrated in FIG. 1C. Hence, in some embodiments herein, the number of binding domains comprising an ACE protein of a polypeptide construct comprising a multimeric IgA or IgM scaffold can be higher than the number of Fc polypeptides of such construct, i.e., when the additional binding domain(s) is coupled to a tailpiece assembly and/or a J-chain.

In some embodiments, provided herein is an ACE2 decoy polypeptide construct comprising one or more linkers, such as a first linker, second linker, etc., and wherein each of such linker is a peptide linker, each comprising up to 50 amino acids, such as being from about 5 to about 25 amino acids in length. Such linker can be used to couple a binding domain to an Fc polypeptide chain, for example.

In some embodiments, an ACE2 decoy polypeptide construct of the present disclosure binds a spike protein of a SARS associated virus, including SARS-coronavirus (SARS-CoV) or SARS-coronavirus 2 (SARS-CoV-2).

One embodiment of the present disclosure provides an ACE2 decoy polypeptide construct capable of binding a spike protein of one or more SARS-CoV-2 variants, wherein such variants include the alpha, beta, gamma, delta, mu, eta, iota, kappa, lambda, epsilon, theta, zeta, and/or the omicron variant, or any other variant and/or subvariants (e.g., omega subvariants BA.3, BA.4, BA.5, etc.) expressing a spike protein. Hence, in various embodiments, an ACE2 decoy polypeptide construct has a binding affinity for spike proteins of two, three, four, five, or more different variants with a K_{D} of ≤10⁻⁹ M. Such a polypeptide construct can also be referred to herein as variant-agnostic.

In various embodiments, such variant-agnostic ACE2 decoy polypeptide construct herein is the decavalent construct v36708 or v36709 comprising a pentameric IgM Fc scaffold. In some embodiments, the variant-agnostic ACE2 decoy polypeptide construct is v36708.

Another embodiment of the present disclosure provides a pharmaceutical composition comprising an ACE2 decoy polypeptide construct of this disclosure and a pharmaceutically acceptable carrier and/or a diluent. In various instances, such pharmaceutical composition can be formulated into a dosage from for, e.g., intranasal administration to a subject (e.g., a human or a rodent).

Another embodiment of the present disclosure provides a host cell comprising a nucleic acid molecule that encodes an ACE2 decoy polypeptide construct of the present disclosure. Such host cell can further be able to express the polypeptide construct, or a functional subunit thereof, such as ACE2 protein coupled to an Fc polypeptide.

Hence, one embodiment of the present disclosure provides a method of producing an ACE2 decoy polypeptide construct of the present disclosure, the method comprising (i) culturing a host cell culture comprising a host cell comprising a nucleic acid molecule that encodes the polypeptide construct, (ii) expressing the polypeptide construct in the host cell, and (iii) recovering the polypeptide construct from the host cell culture. In some cases, such method further comprises purifying the recovered polypeptide construct using, e.g., an appropriate chromatography method known in the art and as described herein, to yield a purity of at least 90%, 95%, or at least about 97% for the expressed construct.

Another embodiment of the present disclosure relates to a method of administering to a subject in need thereof an effective amount of an ACE2 decoy polypeptide construct of the present disclosure. In some embodiments, administering the polypeptide construct comprises parenteral, oral, mouth inhalation, nasal inhalation, intranasal or pulmonary administration. In certain embodiments, administering the polypeptide construct comprises using a medical device that allows for mouth inhalation, nasal inhalation, intranasal or pulmonary administration. As further described herein, in some embodiments, the polypeptide construct can be administered to the subject either (i) therapeutically following a diagnosis of a viral infection caused by a SARS associated virus, or (ii) prophylactically as the subject may be considered of being at risk of becoming infected with the SARS associated virus. In some embodiments, the SARS associated virus is a SARS-CoV or SARS-CoV-2 associated virus and can cause a disease in the subject selected from SARS-CoV-2 infection is Coronavirus Disease 2019 (COVID-19), Multisystem inflammatory syndrome in adults (MIS-A) and Multisystem inflammatory syndrome in children (MIS-C).

In various embodiments herein, the subject being treated with an ACE2 decoy polypeptide construct of this disclosure is a human, a non-human primate, or a rodent.

### FIGURES OF THE DISCLOSURE

The implementations disclosed herein are illustrated by way of example, and not by way of limitation, in the accompanying drawings. The description and drawings are only for the purpose of illustration and as an aid to understanding and are not intended as a definition of the limits of the compounds, conjugates, compositions, and methods of the present disclosure.

FIGS. 1A-1M show illustrations of ACE2 decoy polypeptide constructs according to embodiments of the present disclosure having varying valency for ACE2 based on IgM (FIGS. 1A-1C), IgA (FIGS. 1D-1F) and IgG (FIGS. 1G-1M) domains and/or scaffolds.

FIGS. 2A-2C show dissociation constants K_{D} (FIG. 2A) as well as kinetic parameters including rate constants K_{ON} (FIG. 2B) and K_{OFF} (FIG. 2C) as determined in an SPR experiment of ACE2 decoy polypeptide constructs described herein of varying valency for ACE2 and with either WT or catalytic knockout (referred to herein as “KO”) ACE2 proteins, to immobilized SARS-CoV-2 Spike protein. ACE2 valency (given as a number in parentheses) for the tested constructs were as follows: v34803 (1), 34801 (2), v34797 (4) and v34799 (10), and for the ACE2 catalytic knockout (KO) variants: 34804 (KO, 1), 34802 (KO, 2), v34798 (KO, 4) and v34800 (KO, 10). Data for multiple different immobilization levels are also shown.

FIGS. 3A-3D show on-cell binding data for ACE2 decoy polypeptide constructs of the present disclosure of varying valency and with (i) WT ACE2 (v34803 (1), 34801 (2), v34797 (4) and v34799 (10)), (ii) KO ACE2 (34804 (KO, 1), 34802 (KO, 2), v34798 (KO, 4) and v34800 (KO, 10)), as well as (iii) recombinant ACE2 and (iv) a negative control as positive and negative control samples, respectively, to SARS-CoV-2 Spike protein transfected HEK2936e cells. Data are shown for binding of the tested polypeptide constructs to wildtype (WT) Spike protein (FIG. 3A), B.1.1.7 variant Spike protein (FIG. 3B) and B.1.351 variant Spike (FIG. 3C). FIG. 3D shows a summary of the calculated EC₅₀ binding data for the tested constructs against the WT and 2 variant (B.1.1.7 and B.1.351) spike proteins.

FIGS. 4A-4I show data for (i) ACE2 decoy polypeptide constructs of varying valency and with WT and KO ACE2 as well as (ii) for three clinical benchmarks (bamlanivimab, REGN10933, REGN10987) for their ability to disrupt the Spike-ACE2 interaction between SARS-CoV-2 and target cells as assessed by a mesoscale discovery (MSD) mutliplexed assay. Inhibition curves for varying construct concentrations are shown for WT Spike receptor binding domain (RBD) (FIG. 4A), WT complete Spike protein (FIG. 4B), B.1.1.7 Spike RBD (FIG. 4C), B.1.1.7 complete Spike protein (FIG. 4D), B.1.351 Spike RBD (FIG. 4E), B.1.351 complete Spike protein (FIG. 4F), P.1 Spike RBD (FIG. 4G), and P.1 complete Spike protein (FIG. 4H). FIG. 4I shows the calculated EC₅₀ values for the tested constructs for neutralizing the tested spike proteins and thereby prevent their interaction with ACE2 proteins of target cells.

FIGS. 5A-5C show data from a WT SARS-CoV-2 Spike pseudovirus neutralization assay for (i) ACE2 decoy polypeptide constructs of varying valency and with WT and KO ACE2 as well as (ii) for three clinical benchmarks (bamlanivimab, REGN10933, REGN10987). Data are shown as percent pseudovirus inhibition (% inhibition, FIG. 5A) and in relative light units (RLU, FIG. 5B). FIG. 5C shows the EC₅₀ values calculated for the tested constructs for the Spike pseudovirus neutralization assay.

FIGS. 6A-6H show results obtained from an in vivo study that tested the IgM-based decavalent ACE2 decoy antibody construct v36708 as well as the bivalent IgG-based ACE2 decoy antibody construct v36710 at different doses (both constructs tested at 0.1, 1, and 10 mg/kg) using a Syrian hamster model of a SARS-CoV-2 Delta variant infection. FIGS. 6A-6B show the impact of intranasal treatment with v36708 (FIG. 6A) or v36710 (FIG. 6B) on body weight change after viral challenge. Error bars represent standard error of the mean. FIGS. 6C-6D show the impact of ACE2 decoy treatment with v36708 (FIG. 6C) or v36710 (FIG. 6D) on viral RNA quantified from hamster oral swab at days 2, 3, 4, and 5 post-challenge. Data were plotted as box-and-whisker plot and the dotted line represents the limit of detection (LOD=5 viral gRNA copies/mL (Log₁₀)). FIGS. 6E-6F show the impact of ACE2 decoy treatment using v36708 (FIG. 6E) or v36710 (FIG. 6F) on viral titres detected in BAL and lung tissue 5 days after infection (**** indicates significant differences with p<0.0001). Data were plotted as box-and-whisker plot and the dotted line represents the limit of detection (LOD=3.1 Log₁₀ (PFU/ml BAL) and 2.5 Log₁₀ (PFU/g lung tissue). Histopathological analysis of hematoxylin-eosin stained hamster lungs at 5 days post infection (FIG. 6G) revealed percent of area of lung exhibiting pathology due to SARS-CoV-2 Delta infection was lower in v36708 treated vs IgM isotype control dose-matched at 10 mg/kg (FIG. 6H). Histology scores are represented as heat map for these two test groups where 1=mild, 2=moderate, and 3=severe pathology.

### DETAILED DESCRIPTION

### I. Introduction

In various embodiments, the present disclosure relates to monovalent and multivalent ACE2 decoy polypeptide constructs comprising one or more ACE2 proteins (either wildtype (wt) or variants thereof), respectively, and which are capable of binding one or more viral spike protein(s) based on their ACE2 valency. Such polypeptide construct can generally comprise (i) an immunoglobulin Fc domain or a multimeric Fc scaffold and (ii) an ACE2 protein, wherein the ACE2 protein is capable of binding a viral spike protein and is coupled to an Fc polypeptide of the Fc domain or Fc scaffold. In various embodiments, a polypeptide construct of the present disclosure is multivalent, i.e., capable of binding to two or more spike proteins. In some embodiments, a multivalent polypeptide construct of this disclosure can bind to two or more spike proteins that belong to different virus variants or subvariants, e.g., different variants of a coronavirus such as SARS-CoV-2. Such constructs, e.g., those that are ACE2 multivalent and comprise ACE2 proteins with wildtype spike protein binding domains having a K_{D} of ≤10⁻⁹ M for spike proteins of two or more virus strains, can be referred to herein as variant-agnostic constructs.

Further described herein are pharmaceutical compositions comprising one or more ACE2 decoy polypeptide constructs, as well as methods for producing and using such constructs and compositions, e.g., for the prophylaxis and/or treatment of a viral infection in a subject.

Unless defined otherwise herein, all technical and scientific terms used herein have the same meaning as commonly understood by one of ordinary skill in the art.

As used herein, the terms “polypeptide construct,” “spike protein binding molecule,” “soluble spike protein binding molecule” and “ACE2 decoy polypeptide construct” can be used interchangeably and refer to a molecule herein that is capable of binding a spike protein of a virus and capable of intercepting the binding of the spike protein of the virus particle with a cell surface-bound ACE2 molecule. Such property of intercepting and preventing the interaction between a virus particle and a target cell can be referred to as a “decoy” function, and thus the multivalent constructs of this disclosure can also be referred to as “decoy constructs, e.g., “ACE2 decoy polypeptide constructs.”

The term “about,” as used herein in the context of a numerical value or range, generally refers to ±10%, 9%, 8%, 7%, 6%, 5%, 4%, 3%, 2%, or 1% of the numerical value or range recited or claimed, unless otherwise specified. In various embodiments, the term “about” refers to an approximately ±10% variation from a given value. In other embodiments, the term “about” refers to an approximately ±5% variation from a given value. In yet other embodiments, the term “about” refers to an approximately ±1% variation from a given value. It is to be understood that such a variation is always included in any given value provided herein, whether it is specifically referred to or not.

The use of the word “a” or “an” when used herein in conjunction with the term “comprising” can mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a construct, a composition, use or method, denotes that additional elements and/or method steps can be present, but that these additions do not materially affect the manner in which the recited construct, composition, method or use functions. The term “consisting of,” when used herein in connection with a construct, a composition, use or method, excludes the presence of additional elements and/or method steps. A construct, composition, use, or method described herein as comprising certain elements and/or steps can also, in certain embodiments consist essentially of those elements and/or steps, and in other embodiments consist of those elements and/or steps, whether or not these embodiments are specifically referred to.

The terms “subject” and “patient” as used herein refer to an animal in need of treatment. An animal in need of treatment can be a human or a non-human animal, such as a mammal, bird, or fish. In certain embodiments, the subject or patient is a mammal. In some embodiments, the subject is a human. In other cases, the subject is a rodent or a non-human primate.

An “effective amount” of a construct described herein in respect of a particular result to be achieved is an amount sufficient to achieve the desired result. For example, an “effective amount” of a polypeptide construct disclosed herein when referred to in respect of neutralizing an interaction of a cell surface-bound ACE2 protein and a spike protein of a virus, refers to an amount of polypeptide construct sufficient to produce a measurable neutralizing effect.

It is to be understood that the positive recitation of a feature in one embodiment, serves as a basis for excluding the feature in an alternative embodiment. In particular, where a list of options is presented for a given embodiment or claim, it is to be understood that one or more option can be deleted from the list and the shortened list can form an alternative embodiment, whether or not such an alternative embodiment is specifically referred to.

As used herein, the term “ACE2” refers to angiotensin converting enzyme 2 (ACE2), which has been identified as the point of entry into host cells for viral particles utilizing their spike protein for ACE2 binding. It is further known that ACE2 is an important enzyme in the renin-angiotensin-aldosterone system (RAAS), scarcely present in the circulation, but widely expressed in organs and able to regulate blood pressure and fluid balance. ACE2 is generally widely expressed throughout a mammalian (e.g., human) body, including epithelia of the respiratory system (e.g., tracheal and bronchial epithelial cells, alveolar epithelial cells, type 2 pneumocytes), the cardiovascular system (e.g., endothelium of coronary arteries, myocites, epicardial adipocites, vascular endothelial, and smooth cells), the gastrointestinal tract (e.g., esophagus keratinocytes, gastrointestinal epithelial cells, intestinal epithelial cells, duodenum, small intestine, rectum), the urogenital system (e.g., kidney proximal tubules, bladder urothelial cells, luminal surface of tubular epithelial cells, testis, seminal vesicle), as well as in the liver and gallbladder and in the nervous system. Any of such cell types, as well as other cell types known to express relevant levels of ACE2, are encompassed by the term “target cell” used herein in the context of an interaction between a viral particle comprising a spike protein and its potential target cell for entry and subsequent viral replication.

It is contemplated that any embodiment discussed herein can be implemented with respect to any method, use or composition disclosed herein, and vice versa.

All publications, patents and patent applications mentioned in this specification are herein incorporated by reference into the specification to the same extent as if each individual publication, patent, or patent application was specifically and individually indicated to be incorporated herein by reference.

### II. Polypeptide Constructs

In various embodiments, the present disclosure describes polypeptide constructs capable of binding a viral spike protein. The viral spike protein can belong to a SARS associated virus, or another virus, as described herein. In various embodiments, a polypeptide construct of the present disclosure generally comprises (i) an immunoglobulin Fc domain or Fc scaffold and (ii) a binding domain comprising or consisting of an ACE2 protein. The ACE2 protein of the binding domain can be capable of binding the viral spike protein.

In various embodiments, a polypeptide construct of the present disclosure comprises a binding domain comprising the extracellular portion of a full-length ACE2 protein. Hence, the ACE2 proteins used in the polypeptide construct of the present disclosure can lack the membrane anchor sequence and can therefore be characterized as soluble, unlike a full-length ACE2 protein which may be membrane-anchored. A polypeptide construct of this disclosure can act as a competitive interceptor of virus particles, e.g., SARS associated viruses, comprising spike proteins by preventing binding of the virus particle via its spike protein to a surface-bound, full-length ACE2 receptor of a cell targeted by the virus. Accordingly, the polypeptide constructs of the present disclosure are also referred to herein as decoy polypeptide constructs or decoy molecules, eluting to their ability to intercept and bind SARS associated viral particles and hence prevent them from binding to ACE2 on the surface of cells targeted by the virus for entry and multiplication.

In this context, described herein are polypeptide constructs that can be used to prevent and/or treat an infection in a subject caused by, e.g., a SARS associated virus, or another virus, that utilizes ACE2 as a receptor for cell entry. Since enzymatic activity of the ACE2 protein may not be required for spike protein binding, and therefore for the decoy effect of a polypeptide construct herein, both enzymatically active and non-active (knockout or KO) ACE2 proteins are contemplated and used in the constructs as further described herein. Additional embodiments herein contemplate modified ACE2 proteins having increased thermal stability compared to wildtype ACE2 proteins.

As described herein, a polypeptide construct of this disclosure can comprise one or more, two or more, three or more, four or more, five or more, six or more, eight or more, ten or more, or twelve or more binding domains, wherein each of such binding domain can comprise or, in some embodiments, consist of an ACE2 protein, or a portion thereof, as further described herein.

### A. Binding Domains Comprising or Consisting of an ACE2 Protein

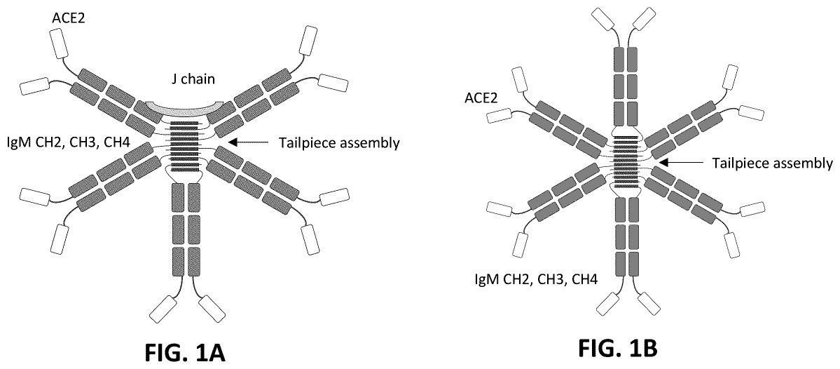

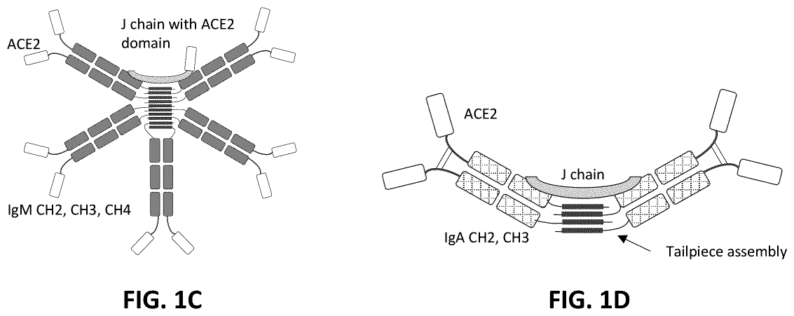

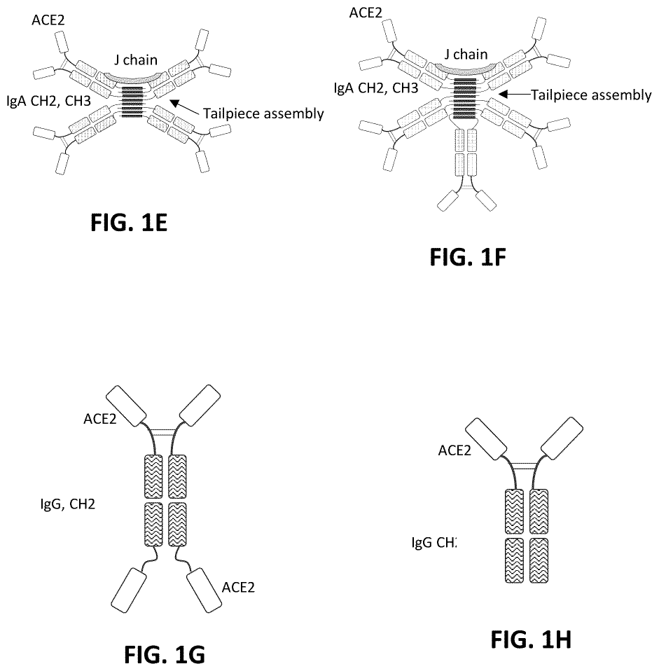

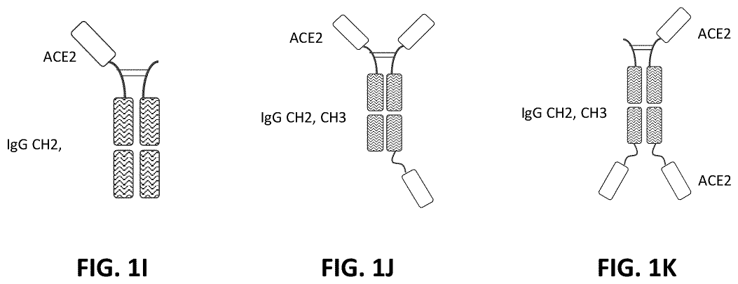

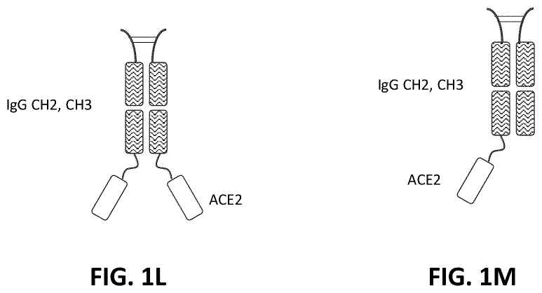

A polypeptide construct of this disclosure can be monovalent (see, e.g., FIG. 1I and FIG. 1M) or multivalent (see, e.g., FIGS. 1A-1H) with respect to ACE2. A multivalent polypeptide construct of this disclosure can be bivalent, trivalent, tetravalent, hexavalent, octavalent, decavalent, or dodecavalent for ACE2, among other valencies contemplated herein. Hence, a polypeptide construct of the present disclosure can comprise one or more binding domains, wherein each of such one or more binding domains comprises or consists of an ACE2 protein.

Generally, and unless otherwise specified herein, an ACE2 protein of a polypeptide construct herein generally refers to an extracellular portion of a wildtype ACE2 protein, which is generally membrane-bound when consisting of the full length sequence, and can be one that comprises or consists of either (i) the wildtype amino acid sequence set forth in any one of SEQ ID NOs: 2-3, (ii) a fragment of the amino acid sequence set forth in SEQ ID NOs: 2-3, wherein such fragment is still capable of binding a viral spike protein, (iii) an amino acid sequence which has about 80%, 90%, 95%, 97%, 99%, or 100% sequence identity to any of the amino acid sequences set forth in SEQ ID NOs: 2-3, or (iv) a combination of (i)-(iii).

In addition to its one or more binding domains, a polypeptide construct herein comprises an immunoglobulin (Ig) Fc domain or scaffold to which the one or more binding domains are coupled, either directly or via linkers. FIGS. 1A-1M show polypeptide constructs of the present disclosure having various formats (e.g., can be IgG, IgA, or IgM-based) and different valencies for ACE2.

Thus, in various embodiments, provided herein is a polypeptide construct, comprising: (i) at least three binding domains, each binding domain comprising an ACE2 protein capable of binding to a viral spike protein; (ii) at least three dimeric Fc domains, each dimeric Fc domain comprising a first Fc polypeptide and a second Fc polypeptide; and (iii) a tailpiece assembly, wherein: a) at least one of the first and the second Fc polypeptide of each dimeric Fc domain is coupled via its N-terminus to one of the at least three binding domains, and b) the first and second Fc polypeptides of the three dimeric Fc domains are each directly or indirectly coupled to each other via their C-terminal regions (e.g., covalently or non-covalently via their tailpiece sequences), thereby forming the tailpiece assembly.

In various embodiments, such polypeptide construct comprises at least four, five, or six binding domains, wherein each binding domain is coupled to the N-terminus of a first or second Fc polypeptide. A polypeptide construct herein is considered to have full valency for ACE2 when each Fc polypeptide chain is coupled to at least one binding domain comprising an ACE2 protein.

Hence, in various embodiments, a polypeptide construct having three dimeric Fc domains can comprise six binding domains, such that each binding domain is coupled to the N-terminus of a first or second Fc polypeptide, thereby forming a hexavalent polypeptide construct.

In other embodiments, a polypeptide construct herein can comprise more than three dimeric Fc domains, such as four, five or six dimeric Fc domains.

In such embodiments, a polypeptide construct can comprise five dimeric Fc domains. Such five dimeric Fc domains can belong to a pentameric IgM Fc scaffold or IgA scaffold. Such a construct can have a full valency for ACE2 when it comprises at least ten binding domains each comprising an ACE2 protein, and when each binding domain is coupled to the N-terminus of a first or second Fc polypeptide of each of the five dimeric Fc domains, thereby forming a decavalent polypeptide construct.

In other cases, a polypeptide construct can comprise six dimeric Fc domains. Such six dimeric Fc domains can belong to a hexameric IgM Fc scaffold or IgA scaffold. Such a construct can have a full valency for ACE2 when it comprises at least twelve binding domains each comprising an ACE2 protein, and when each binding domain is coupled to the N-terminus of a first or second Fc polypeptide of each of the six dimeric Fc domains, thereby forming a dodecavalent polypeptide construct.

In various embodiments, provided herein is a polypeptide construct, comprising: (i) ten binding domains, each binding domain comprising an ACE2 protein capable of binding to a viral spike protein; (ii) five dimeric Fc domains, each dimeric Fc domain comprising a first Fc polypeptide and a second Fc polypeptide; and (iii) a tailpiece assembly, wherein a) each of the ten Fc polypeptides is coupled via its N-terminus to one of the ten binding domains, and b) the first and second Fc polypeptides of the five dimeric Fc domains are each directly or indirectly coupled to each other via their C-terminal regions (e.g., covalently or non-covalently via their tailpiece sequences), thereby forming the tailpiece assembly. In various embodiments, the C-terminal region of each Fc polypeptide that forms the tailpiece assembly comprises or consists of a tailpiece sequence. In some embodiments, such tailpiece sequence can have an amino acid sequence having at least about 90%, 95%, or 100% sequence identity to the amino acid sequence set forth in SEQ ID NO: 9. At least one of the binding domains can comprise an ACE2 protein comprising or consisting of an amino acid sequence having at least about 80%, 90%, 95%, 97%, 99%, or 100% sequence identity to any of the amino acid sequences set forth in SEQ ID NOs: 1-3. In various other embodiments, each binding domain comprises an ACE2 protein comprising or consisting of an amino acid sequence having at least about 80%, 90%, 95%, 97%, 99%, or 100% sequence identity to any of the amino acid sequences set forth in SEQ ID NOs: 1-3. In various embodiments, when an ACE2 protein comprises one or more amino acid modifications, such modifications are located outside the domain responsible for interaction with a viral spike protein.

In yet other embodiments, each binding domain comprises or consists of an ACE protein comprising or consisting of any one of the amino acid sequences set forth in SEQ ID NOs: 1-3. In some embodiments, each binding domain of the decavalent construct comprises or consists of the amino acid sequence set forth in SEQ ID NO: 2. In some embodiments, each binding domain of the decavalent construct comprises or consists of the amino acid sequence set forth in SEQ ID NO: 3.

In some embodiments, at least one of the ACE2 proteins used in a polypeptide construct herein can be a modified ACE2 protein comprising one or more amino acid modifications relative to a wildtype sequence. Such one or more amino acid modifications can comprise one or more amino acid substitutions, one or more amino acid insertions, one or more amino acid deletions, or a combination thereof. In various cases, the one or more amino acid modifications comprise or consist of one or more amino acid substitutions.

Hence, in some embodiments, at least one of the binding domains comprises an ACE2 protein comprising one or more amino acid substitutions relative to any of the amino acid sequences set forth in SEQ ID NOs: 1-3. In such instances, the one or more amino acid substitutions are located outside the region that interacts with the binding domain of the viral spike protein, and thereby not or not materially affect spike protein binding of the construct.

In various embodiments, the one or more amino acid substitutions can reduce or eliminate the catalytic function of the ACE2 protein. Hence, such one or more amino acid substitutions can comprise the catalytic knock-out (“KO”) mutations H374N_H378N relative to the sequence set forth in SEQ ID NO: 1, resulting, e.g., in a catalytic KO sequence of the extracellular domain of ACE2 set forth in SEQ ID NO: 4.

In some embodiments, a polypeptide construct of this disclosure comprises at least one binding domain that comprises or consists of an ACE2 protein which itself comprises or consists of the amino acid sequence set forth in SEQ ID NO: 4.

In yet other embodiments, the one or more amino acid substitutions comprise substitution(s) that increase the stability of the binding domain relative to a binding domain comprising an ACE2 protein without the one or more amino acid substitutions, and wherein the increase in stability is measured as an increase in melting temperature of at least about 1° C., 2° C., 3° C., 4° C., 5° C., 6° C., 7° C., or about 8° C. as measured using differential scanning calorimetry (DSC).

Such one or more amino acid substitutions that can increase the stability (e.g., thermal stability) of an ACE2 protein and thereby enhance the developability of a polypeptide construct are also located outside the binding region involved in spike protein binding, and hence can be at any one or more of the positions 73, 180, 226, 293, 304, 311, 342, 360, 362, 372, 373, 396, 411, 412, 417, 423, 447, 453, 507, 520, 522, 526, 533, 550, 569, 647, 650, 686 and 707, based on the sequence set forth in SEQ ID NO: 1. In some embodiments, such one or more substitutions may introduce cysteine residue(s) to form additional disulfide bonds. Such substitutions can include any one or more of A396C_596C, H373C_A412C, A533C_A550C, A311C_A372C, A304C_T362C_A650C_S707C and V293C_L423C. ACE2 proteins comprising such substitutions can comprise or consist of any one of the sequences set forth in SEQ ID NOs: 44-50. In other embodiments, a modified ACE2 protein can have an increased stability by containing one or more amino acid substitutions that increase non-covalent interactions. Such substitutions can be selected from any one or more of L520I, L73Y, A342V, V447I, V226I, Q526F, Q526Y, H417F, T362I, T453L, V647I, S507L, S411H, M360W, M360F, Q522M, Y180W, S411I, Q526H, T686I and V647L, based on the sequence set forth in SEQ ID NO: 1.

ACE2 proteins comprising such substitutions can comprise or consist of any one of the sequences set forth in SEQ ID NOs: 51-72.

### B. Fc Domains and Fc Scaffolds of Polypeptide Constructs

As further described herein, a polypeptide construct of the present disclosure, in addition to its one more (e.g., 2, 3, 4, 6, 8, 10, 12, or more) binding domains, further comprises an Fc domain or an Fc scaffold. As used herein, the term “Fc domain” generally refers to a single dimeric Fc domain comprising a first and second Fc polypeptide, wherein such first and second Fc polypeptides comprise one or more of CH2, CH3, and/or CH4 domains, e.g., depending on whether the Fc domain is IgG, IgA, or IgM based. Moreover, as used herein, the term “Fc scaffold” generally refers to an Fc structure comprising two or more dimeric Fc domains, e.g., four, five or six dimeric Fc domains as in, e.g., IgM Fc scaffolds or IgA Fc scaffolds, see, e.g., FIGS. 1A-1C and 1E-1F.

In various embodiments of the present disclosure, an Fc domain (e.g., an IgG, IgA, or IgM Fc domain) is a dimeric Fc domain comprising a first and a second Fc polypeptide. In some embodiments, such dimeric Fc domain is homodimeric, e.g., the first and second Fc polypeptide comprise or consist of an identical amino acid sequence. In other cases, a dimeric Fc domain can be heterodimeric, e.g., when the first and second Fc polypeptide each comprise or consist of a different amino acid sequence, e.g., sharing no more than 99%, 97%, 95%, or no more than 90% sequence identity. This can, for example, be the case when both the first and second Fc polypeptide each comprise one or more asymmetric amino acid modification that allow for preferential formation of the heterodimeric Fc domain, compared to the formation of a corresponding homodimeric Fc domain.

In some embodiments, a polypeptide construct disclosed herein can comprise a dimeric Fc domain selected from an IgG Fc domain, an IgA Fc domain, an IgM Fc domain, or a combination thereof. Such combination of one or more of an IgG Fc domain, one or more of an IgA Fc domain, and/or one or more of an IgM Fc domain, can form an Fc scaffold comprising two or more dimeric Fc domains of different Ig classes. In other embodiments herein, a polypeptide constructs disclosed herein comprises an Fc scaffold comprising two or more dimeric Fc domains, wherein such two or more dimeric Fc domains are of the same class, i.e., the two or more dimeric Fc domains are all IgG, IgA, or IgM Fc domains.

Accordingly, a polypeptide construct herein that comprises one or more dimeric Fc domains can comprise, in some embodiments, at least one IgG Fc domain. In some embodiments, a polypeptide construct herein comprises only IgG Fc domain(s), e.g., one or more IgG Fc domain(s). In such instances, and in cases in which a polypeptide construct is multivalent for ACE2, i.e., comprises more than one binding domain comprising an ACE2 protein, such polypeptide construct can comprise an IgG Fc domain wherein each of the two Fc polypeptides comprises a CH2 and a CH3 domain, and wherein each Fc polypeptide can be coupled to one or more than one, e.g., two, binding domains, e.g., one coupled to its N-terminus and another one coupled to its C-terminus. Such embodiments are illustrated, e.g., in FIGS. 1J and 1K.

In other embodiments herein, a polypeptide construct comprising one or more dimeric Fc domains can comprise at least one IgA Fc domain. In such embodiments, a polypeptide construct can comprise only IgA Fc domain(s), e.g., one or more IgA Fc domain(s). Hence, in some embodiments, a polypeptide construct can comprise an IgA Fc scaffold comprising more than one dimeric IgA Fc domain, e.g., two dimeric IgA Fc domains, three dimeric IgA Fc domains, four dimeric IgA Fc domains, or five dimeric IgA Fc domains. Such embodiments of the present disclosure are illustrated in FIGS. 1D-1F, respectively. Hence, in instances where a polypeptide construct comprises an IgA Fc scaffold, such scaffold can comprise a tailpiece assembly and/or a J-chain, thereby linking the C-termini of the dimeric Fc domains together. In various embodiments, the first and second Fc polypeptides of the dimeric IgA Fc domains that can make up the multimeric IgA Fc scaffold can each comprise a CH2 and/or a CH3 domain. In some instances, the first and second Fc polypeptides each comprise a CH2 and a CH3 domain.

In various embodiments, a polypeptide construct described herein comprises an IgM Fc scaffold comprising two or more dimeric IgM Fc domains. In some instances, such IgM Fc scaffold comprises three, four, five, six, or more dimeric IgM domains. As described herein, at least one Fc polypeptide, e.g., at least a first or second Fc polypeptide, of an IgM scaffold can comprise one or more of a CH2 domain, CH3 domain, and a CH4 domain. In various cases, each Fc polypeptide of an IgM scaffold of a polypeptide construct described herein comprises a CH2 domain, a CH3 domain, and a CH4 domain. Such CH2 domain, CH3 domain, and CH4 domains of the one or more IgM Fc polypeptides can each comprise or consist of an amino acid sequence that has at least about 80%, 90%, 95%, 97%, 99%, or 100% sequence identity to the amino acid sequences set forth in SEQ ID NOs: 5-7, respectively. In various embodiments, the CH2 domain, CH3 domain, and CH4 domains of all IgM Fc polypeptides of an IgM scaffold comprise or consist of the amino acid sequences set forth in SEQ ID NOs: 5-7, respectively. In other embodiments, the CH4 domains of the IgM Fc polypeptides herein comprise or consist of the amino acid sequence set forth in SEQ ID NO: 8 comprising the sequence set forth in SEQ ID NO: 7 in addition to a tailpiece sequence set forth in SEQ ID NO: 9.

In various embodiments, a polypeptide construct described herein comprises an IgM Fc scaffold comprising five dimeric Fc domains. Such five dimeric Fc domains can be heterodimeric or homodimeric. In cases of homodimeric Fc domains, the first and second Fc polypeptides of the five dimeric Fc domains comprise or consist of an identical amino acid sequence. Hence, in some embodiments, an IgM scaffold of a polypeptide construct herein can comprise five homodimeric IgM Fc domains, wherein each of the ten Fc polypeptides comprises or consists of an identical amino acid sequence. Examples of such polypeptide constructs are the constructs v36708 and 36709. FIG. 1A illustrates the structure of a polypeptide construct comprising a pentameric IgM scaffold, a tailpiece assembly formed by the C-terminal regions (e.g., tailpiece sequences) of the Fc polypeptides, and a J-chain. In such and some other embodiments of this disclosure, each Fc polypeptide, e.g., each first and second Fc polypeptide of each dimeric Fc domain, comprises, from N- to C-terminus, a CH2 domain, a CH3 domain and a CH4 domain, and wherein each Fc polypeptide is coupled via its N-terminus to a binding domain comprising an ACE2 protein, thereby making this a decavalent construct capable of binding one or more viral spike proteins, e.g., on the surface of one or more viral particles, as described herein.

In some embodiments, a polypeptide construct described herein comprises an IgM Fc scaffold comprising six dimeric Fc domains. Such six dimeric Fc domains can be heterodimeric or homodimeric. In cases of homodimeric Fc domains, the first and second Fc polypeptides of such six dimeric Fc domains comprise or consist of an identical amino acid sequence. Hence, in some embodiments, an IgM scaffold of a polypeptide construct herein can comprise six homodimeric Fc domains, wherein each Fc polypeptide comprises or consists of an identical amino acid sequence. FIG. 1B illustrates the structure of a polypeptide construct comprising a hexameric IgM scaffold and a tailpiece assembly formed by the C-terminal regions (e.g., tailpiece sequences) of the Fc polypeptides. In such and some other embodiments of this disclosure, each Fc polypeptide, e.g., each first and second Fc polypeptide of each dimeric Fc domain, comprises, from N- to C-terminus, a CH2 domain, a CH3 domain and a CH4 domain, and wherein each Fc polypeptide is coupled via its N-terminus to a binding domain comprising an ACE2 protein, thereby making this a dodecavalent construct capable of binding one or more viral spike proteins, e.g., on the surface of one or more viral particles, as described herein.

As described herein, in some embodiments, a polypeptide construct comprises an Ig Fc scaffold, such as an IgM scaffold, that comprises a J-chain. Such J-chain can comprise or consist of an amino acid sequence having at least about 80%, 90%, or 100% sequence identity to the amino acid sequence set forth in SEQ ID NO: 10 or SEQ ID NO: 12. In some cases, a J-chain can also be coupled to a binding domain comprising an ACE2 protein as described herein. Such an embodiment is illustrated in FIG. 1C.

Two or more of the various structural components, e.g., binding domain(s), Fc polypeptide(s), etc., of a polypeptide construct described herein can be covalently or non-covalently coupled to each other. In cases of a covalent coupling of one component to another, e.g., a binding domain to an Fc polypeptide, such coupling can occur either (i) directly, i.e., without a linker moiety, or (ii) indirectly via a linker. In some embodiments, at least one or all of the binding domains of a construct herein are each coupled directly, i.e., without a linker, to an Fc polypeptide. In other embodiments, at least one or all of the binding domains of a construct herein are each coupled indirectly, i.e., via a linker, to an Fc polypeptide.

### C. Linkers and Tailpiece Sequences

In various embodiments, a polypeptide construct of the present disclosure can comprise one or more linkers. Such one or more linkers can be peptide linkers, e.g., comprising or consisting of an amino acid sequence having from about 5 to about 50 amino acids.

Hence, in some embodiments, at least one of the binding domains of a construct herein is coupled to the N-terminus of an Fc polypeptide via a first linker. In some instances, all binding domains of a construct herein can be coupled to their respective Fc polypeptides via a first linker, i.e., all linkers coupling the binding domains to the Fc polypeptides have the same first linker amino acid sequence.

In embodiments herein in which a polypeptide construct comprises a multimeric Fc scaffold comprising a tailpiece assembly (e.g., penta- or hexameric IgM or IgA Fc scaffolds), at least one of the Fc polypeptides can comprise a C-terminal region that forms at least a part of the tailpiece assembly. In some cases, all Fc polypeptides of a construct herein comprise a C-terminal region that together can form the tailpiece assembly, e.g., by covalently (e.g., through disulfide bonds) or non-covalently coupling the C-terminal regions of the Fc polypeptides to one another, thereby forming the tailpiece assembly. In some embodiments, such C-terminal region of the IgM Fc polypeptides is a tailpiece sequence that can comprise or consist of an amino acid sequence having at least about 80%, 90%, or 100% sequence identity to the amino acid sequence set forth in SEQ ID NO: 9. In some instances, the tailpiece sequences of a polypeptide construct comprise or consist of the amino acid sequence set forth in SEQ ID NO: 9.

### D. Certain Embodiments of Multivalent Polypeptide Constructs

In various embodiments, provided herein is a polypeptide construct comprising: (i) ten binding domains, each binding domain comprising or consisting of an ACE2 protein capable of binding to a viral spike protein, and comprising or consisting of an amino acid sequence having at least about 80%, 90%, 95%, 97%, 99%, or 100% sequence identity to any of the amino acid sequences set forth in SEQ ID NOs: 2-4; (ii) a pentameric IgM scaffold comprising five dimeric Fc domains, each dimeric Fc domain comprising a first Fc polypeptide and a second Fc polypeptide, and each first and second Fc polypeptide comprising a tailpiece sequence; a tailpiece assembly; and a J-chain, wherein (a) each of the ten Fc polypeptides is coupled via its N-terminus to one of the ten binding domains, thereby forming ten Fc-binding domain fusion polypeptides, and (b) the C-terminal tailpiece sequences of the Fc polypeptides form the tailpiece assembly. Each Fc polypeptide can further comprise IgM CH2, CH3 and CH4 domains. In some of these embodiments, at least one of the ten “Fc-binding domain” fusion polypeptides comprises or consists of an amino acid sequence having at least about 80%, 90%, 95%, 97%, 99%, or 100% sequence identity to the amino acid sequence set forth in SEQ ID NO: 28 or SEQ ID NO: 30. Hence, in some instances, a polypeptide construct herein can be the construct v36708 or v36709. FIG. 1A illustrates the structure and configuration of such a construct. In various embodiments, a polypeptide construct herein can be the construct v36708.

In other embodiments, provided herein is a polypeptide construct comprising: (i) twelve binding domains, each binding domain comprising or consisting of an ACE2 protein capable of binding to a viral spike protein, and comprising or consisting of an amino acid sequence having at least about 80%, 90%, 95%, 97%, 99%, or 100% sequence identity to any of the amino acid sequences set forth in SEQ ID NOs: 2-4; (ii) a hexameric IgM scaffold comprising six dimeric Fc domains, each dimeric Fc domain comprising a first Fc polypeptide and a second Fc polypeptide, and each first and second Fc polypeptide comprising a tailpiece sequence; a (iii) tailpiece assembly, wherein (a) each of the twelve Fc polypeptides is coupled via its N-terminus to one of the twelve binding domains, thereby forming twelve “Fc-binding domain” fusion polypeptides, and (b) the C-terminal tailpiece sequences of the Fc polypeptides form the tailpiece assembly.

In yet other embodiments, provided herein is a polypeptide construct comprising: (i) ten binding domains, each binding domain comprising or consisting of an ACE2 protein capable of binding to a viral spike protein, and comprising or consisting of an amino acid sequence having at least about 80%, 90%, 95%, 97%, 99%, or 100% sequence identity to any of the amino acid sequences set forth in SEQ ID NOs: 2-4; (ii) a pentameric IgA scaffold comprising five dimeric IgA Fc domains, each dimeric Fc domain comprising a first Fc polypeptide and a second Fc polypeptide comprising IgA CH2 and CH3 domains and a tailpiece sequence; a tailpiece assembly; and a J-chain, wherein (a) each of the ten Fc polypeptides is coupled via its N-terminus to one of the ten binding domains, thereby forming ten “Fc-binding domain” fusion polypeptides, and (b) the C-terminal tailpiece sequences of the Fc polypeptides form the tailpiece assembly. FIG. 1F illustrates the structure and configuration of such a construct.

As described herein, the polypeptide constructs disclosed herein can be capable of binding to a viral spike protein, e.g., one that is located on the surface of a viral particle. In various instances, the constructs contemplated herein are virus variant-agnostic as described herein, and among other properties that a construct described herein can possess.

### E. Properties of Polypeptide Constructs

In various embodiments, provided herein is a polypeptide construct capable of binding to a viral spike protein, e.g., one that is located on the surface of a viral particle. The viral spike protein can be from a SARS associated virus. The SARS associated virus can be SARS-coronavirus (SARS-CoV) or SARS-coronavirus 2 (SARS-CoV-2). In various cases, the polypeptide construct is multivalent. In some instances, the multivalent polypeptide construct can be a decavalent IgM-based construct as described herein.

In some embodiments, such multivalent polypeptide construct can have an apparent K_{D} for binding to a viral spike protein of ≤10⁻⁸ M, ≤10⁻⁹ M, or ≤10⁻¹⁰ M. In various cases, such binding is virus variant agnostic, i.e., the construct can have an apparent K_{D} of ≤10⁻⁹ M or ≤10⁻¹⁰ M for two or more spike proteins from two or more different virus variants, such as from one or more of the SARS-CoV-2 variants alpha, delta, omicron, etc., as described herein. The binding affinity of a construct to various viral spike proteins can be measured using methods known in the art, e.g., SPR, as further described herein.

In various embodiments, a multivalent polypeptide construct, e.g., those that are deca- or dodecavalent for ACE2 and IgM or IgA based, can have an EC₅₀ value for neutralizing the interaction between an ACE2 receptor which is surface-bound to a target cell (e.g., a tracheal or bronchial epithelial cell, as further described herein) and a spike protein of a SARS-CoV-2 viral particle of ≤10⁻⁹ M, ≤5×10⁻¹⁰ M, or ≤10⁻¹⁰ M, wherein the SARS-CoV-2 viral particle is selected from any one or more of the following SARS-CoV-2 variants: WT, B.1.1.7, B.1.351, B.1.617.2, B.1.1.529 and P.1. In such instances, a multivalent polypeptide construct, e.g., those that are deca- or dodecavalent for ACE2 and IgM based, can have an EC₅₀ value for neutralizing the interaction between ACE2 surface-bound to a target cell and a spike protein of a SARS-CoV-2 viral particle of ≤10⁻⁹ M. In some instances, a multivalent polypeptide construct, e.g., those that are deca- or dodecavalent for ACE2 and IgM based, can have an EC₅₀ value for neutralizing the interaction between ACE2 surface-bound to a target cell and a spike protein of a SARS-CoV-2 viral particle of ≤5×10⁻¹⁰ M. The ability of constructs described herein to neutralize the interaction of viral spike proteins with target cell surface-bound ACE2 can be assessed, e.g., in vitro using an electrochemiluminescence-based multiplex immune assay.

In further embodiments, a multivalent polypeptide construct, e.g., those that are deca- or dodecavalent for ACE2 and IgM based, can have an EC₅₀ value, given as %-inhibition, for neutralizing the interaction of cell surface-bound ACE2 and a spike protein of a SARS-CoV-2 pseudotyped virus, e.g., lentivirus, of ≤10⁻⁹ M, ≤10⁻¹⁰ M, ≤10⁻¹¹ M, or ≤10⁻¹² M. Such neutralization can be assessed using, e.g., a SARS-CoV-2 Pseudovirus Neutralization Assay quantifying the inhibition of pseudoviral entry into ACE2 expressing target cells in the presence of the polypeptide constructs tested. In some instances, a decavalent IgM-based polypeptide construct described herein can have an EC₅₀ value for neutralizing the interaction of cell surface-bound ACE2 and a spike protein of a SARS-CoV-2 pseudotyped lentivirus of ≤10⁻¹⁰ M.

Hence, in various embodiments herein, provided are multivalent polypeptide constructs having virus-neutralizing activity in vitro and in vivo and that can show surprisingly superior activity over conventional antibody-based therapeutics currently used in the clinic for the treatment of coronavirus-induced diseases, such as COVID-19. Such superior activity, e.g., in instances of a decavalent polypeptide construct as described herein, can—without being bound by any theory—induced by its increased avidity based on the decavalent format compared to that of conventional drugs currently in use which can be mono- or bivalent. Hence, in various cases, the superior performance of the constructs described herein, e.g., EC₅₀ for neutralizing spike-ACE2 interaction between virus and target cells, is due to their enhanced avidity rather than by using a modified ACE2 protein engineered to possess increased affinity against a viral spike protein. The use of ACE2 proteins containing wildtype interface residues that interact with viral spike proteins may also explain the surprising activity of the constructs described herein against multiple different coronavirus strains (i.e., their variant-agnostic activity), e.g., when compared to molecules currently used in the clinic, as such constructs described herein may not distinguish between virus variants as long as such variants utilize ACE2 for cell entry. Furthermore, as the ACE2 interface that is used in the constructs described herein mimics the cellular ACE2 receptor the virus encounters when interacting with target cells, the potential for selection of escape mutants can be significantly reduced when compared to the use of anti-spike binding domains engineered for enhanced affinity, thus, a general issue that vaccines and other conventional therapeutic approaches based on anti-spike monoclonal antibodies or affinity engineered ACE2 decoy molecules can cause.

As described herein, some of the polypeptide constructs presently disclosed can comprise one or more ACE2 proteins comprising catalytic knockout (KO) mutations, which can essentially render such ACE2 protein(s) inactive for their endogenous proteolytic activity. Hence, in some cases, and without being bound by any theory, it is contemplated herein that a polypeptide construct comprising one or more KO ACE2 proteins may provide an improved safety profile when administered in a therapeutically effective amount to a subject and compared to a corresponding polypeptide construct that does not contain such KO ACE2 proteins.

In certain other embodiments, contemplated herein are polypeptide constructs comprising an ACE2 protein modified for increased affinity for one or more virus-specific spike proteins. Furthermore, the mono- and multivalent IgG, IgA, and IgM-based polypeptide constructs of the present disclosure can, in yet other embodiments, have other molecules such as other protein(s) attached to its Fc domain and/or Fc scaffold instead of one or more ACE2 proteins. Such other molecules may also enable a construct to intercept a viral particle and reduce or prevent it from binding to its target cell using a variety of mechanisms.

Further provided herein are nucleic acid molecules encoding the polypeptide constructs of the present disclosure. Host cells, as further described herein, for producing the polypeptide constructs of the present disclosure are also contemplated herein.

### III. Pharmaceutical Compositions

Further provided herein are pharmaceutical compositions comprising one or more of the mono- or multivalent polypeptide constructs described herein. In various cases, such pharmaceutical composition can further comprise a pharmaceutically acceptable excipient, carrier, buffer, stabiliser, or other materials well known to those skilled in the art. Such materials are generally non-toxic and do not interfere with the efficacy of the active ingredient (i.e., polypeptide construct). The precise nature of a carrier or other material can depend on the route of administration. Hence, a pharmaceutical composition herein can be formulated for various used and administration routes, e.g., for oral, intravenous, cutaneous, subcutaneous, intranasal, intramuscular, or intraperitoneal administration routes.

In various embodiments, a pharmaceutical composition comprising a polypeptide construct of the present disclosure, e.g., on that is deca- or dodecavalent for ACE2 and IgM based, is administered intranasally. Such intranasal administration can comprise using a liquid solution comprising the polypeptide construct, and, in some instances, a delivery device that can aid in administration, e.g., by producing a spray which can be applied directly into the nose of a subject.

In other cases, a pharmaceutical composition is formulated for oral administration, which can be in tablet, capsule, powder, or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose or other saccharide solution or glycols such as ethylene glycol, propylene glycol or polyethylene glycol can be included.

For intravenous, cutaneous, or subcutaneous injection of a pharmaceutical composition comprising a polypeptide construct, the active ingredient (i.e., polypeptide construct) can be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

Generally, for polypeptide constructs according to the present disclosure that are administered to a subject as a pharmaceutical composition, administration is preferably in a “therapeutically effective amount” that is sufficient to show benefit to the individual, as further described herein. A “prophylactically effective amount” can also be administered, when sufficient to show benefit to the individual, wherein such individual may not have been diagnosed with an applicable disease, e.g., viral infection, but may be regarded as being at risk for developing such disease, e.g., infection.

The actual amount administered to a subject, and rate and time-course of administration, can depend on the nature and severity of the disease (e.g., viral infection) being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

### IV. Kits

The present disclosure also provides for kits comprising one or more of the polypeptide constructs described herein, or a pharmaceutical composition comprising such polypeptide construct(s), and instructions for use. In certain embodiments, described herein are kits comprising vectors for expressing a polypeptide construct described herein and instructions for use. In certain other embodiments, described herein are kits comprising host cells comprising a vector for expressing a polypeptide construct and instructions for use. In yet other embodiments, provided herein are kits comprising a purified polypeptide construct and instructions for use. In such cases, the purified polypeptide construct can be lyophilized or provided in a dry form, such as a powder or granules, and the kit can additionally contain a suitable solvent for reconstitution of the lyophilized or dried component(s).

A kit can comprise a container and a label and/or package insert on or associated with the container. The label or package insert contains instructions customarily included in commercial packages of therapeutic products, providing information or instructions about the indications, usage, dosage, administration, contraindications and/or warnings concerning the use of such therapeutic products (e.g., a polypeptide construct described herein). The label or package insert can further include a notice in the form prescribed by a governmental agency regulating the manufacture, use or sale of pharmaceuticals or biological products, which notice reflects approval by the agency of manufacture, for use or sale for human or animal administration. The container can hold a composition comprising a polypeptide construct. In some embodiments, the container can have a sterile access port.

In addition to the container containing a composition comprising a polypeptide construct, the kit can further comprise one or more additional containers comprising other components of the kit. For example, a pharmaceutically acceptable buffer (such as bacteriostatic water for injection) (BWFI), phosphate-buffered saline, Ringer's solution, or dextrose solution), or other buffers or diluents can be included in such kit.

Suitable containers include, for example, bottles, e.g., spray bottles, vials, syringes, intravenous solution bags, and the like. The containers can be formed from a variety of materials such as glass or plastic. If appropriate, one or more components (e.g., a polypeptide construct) of the kit can be lyophilized or provided in a dry form, such as a powder or granules, and the kit can additionally contain a suitable solvent for reconstitution of the lyophilized or dried component(s).

A kit herein can further include other materials desirable from a commercial or user standpoint, such as filters, needles, and syringes.

### V. Methods

The present disclosure provides methods for producing and using the polypeptide constructs described herein.

### A. Methods of Producing a Polypeptide Construct

Provided are methods for preparing the polypeptide constructs described herein. In various cases, a polypeptide construct of the present disclosure can be produced using standard recombinant methods known in the art (see, for example, U.S. Pat. No. 4,816,567 and “Antibodies: A Laboratory Manual,” 2^{nd} Edition, Ed. Greenfield, Cold Spring Harbor Laboratory Press, New York, 2014).

For recombinant production of a polypeptide construct described herein, a poly-nucleotide or set of polynucleotides encoding the polypeptide construct can be generated and inserted into one or more vectors for further cloning and/or expression in a host cell. Polynucleotide(s) encoding the polypeptide construct can be produced by standard methods known in the art (see, for example, Ausubel et al., Current Protocols in Molecular Biology, John Wiley & Sons, New York, 1994 & update, and “Antibodies: A Laboratory Manual,” 2^{nd} Edition, Ed. Greenfield, Cold Spring Harbor Laboratory Press, New York, 2014). As would be appreciated by one of skill in the art, the number of polynucleotides required for expression of the polypeptide construct may be dependent on the format of the polypeptide construct, including, for example, the number of polypeptide chains comprised within a mono-, bi-, tri-tetra-, deca-, or dodecavalent polypeptide construct. In instances in which two or more polynucleotides are used to encode all chains of a polypeptide construct, such two or more polynucleotides can be incorporated into one vector or into more than one vector (e.g., two, three, or more separate vectors).

Generally, for expression, the polynucleotide or set of polynucleotides encoding a polypeptide construct herein can be incorporated into an expression vector together with one or more regulatory elements, such as transcriptional elements, which can be used for efficient transcription of the polynucleotide(s). Examples of such regulatory elements include, but are not limited to, promoters, enhancers, terminators, and polyadenylation signals. One skilled in the art will appreciate that the choice of regulatory elements can be dependent on the host cell selected for expression of the polypeptides of the polypeptide construct and that such regulatory elements can be derived from a variety of sources, including bacterial, fungal, viral, mammalian or insect genes. The expression vector can optionally further contain heterologous nucleic acid sequences that facilitate expression or purification of the expressed polypeptide. Examples include, but are not limited to, signal peptides and affinity tags such as metal-affinity tags, histidine tags, avidin/streptavidin encoding sequences, glutathione-S-transferase (GST) encoding sequences and biotin encoding sequences. The expression vector can be an extrachromosomal vector or an integrating vector. Hence, in some embodiments, the amino acid sequences of the polypeptide chains of an expressed polypeptide construct described herein can comprise a signal peptide sequence. Such signal peptide sequences may vary depending on the expression system and conditions used for producing a polypeptide construct.

Certain embodiments for producing a polypeptide construct of the present disclosure relate to vectors (such as expression vectors) comprising one or more polynucleotides encoding at least a portion of a polypeptide construct described herein. The polynucleotide(s) can be comprised by a single vector or by more than one vector. In some embodiments, the polynucleotides are comprised by a multicistronic vector. Expression vectors that can be used to express polynucleotides include but are not limited to pTT5 and pUC15 cells comprising vectors encoding a polypeptide construct.

Suitable host cells for cloning or expression of the polypeptide constructs described herein include various prokaryotic or eukaryotic cells known in the art. Eukaryotic host cells include, for example, mammalian cells, plant cells, insect cells and yeast cells (such as Saccharomyces or Pichia cells). Prokaryotic host cells include, for example, E. coli, A. salmonicida or B. subtilis cells. In certain embodiments, the host cell used to produce an antibody construct herein is a transient or stable higher eukaryotic cell line, such as a mammalian cell line.

Certain embodiments of the present disclosure relate to a method of making a polypeptide construct comprising culturing a host cell into which one or more polynucleotides encoding the polypeptide construct, or one or more expression vectors encoding the polypeptide construct, have been introduced, under conditions suitable for expression of the polypeptide construct. Such method can further comprise recovering the polypeptide construct from the host cell (or from host cell culture medium). In some cases, such method can further comprise purifying the polypeptide construct.

Hence, in some embodiments, provided herein is a method of producing a polypeptide construct of the present disclosure, the method comprising: (a) expressing the polypeptide construct in a host cell of a host cell culture, the host cell comprising a nucleic acid molecule or a set of nucleic acid molecules encoding the polypeptide construct, and (b) recovering the polypeptide construct from the host cell culture. In some cases, such method further comprises, following step (b), purifying the polypeptide construct such that the polypeptide construct has a chemical purity of ≥95%.

### B. Methods of Use

Further provided herein are methods of using a polypeptide construct of the present disclosure, e.g., for the treatment of a viral infection, such an infection caused by a virus of the SARS-CoV family. Such method of treating a viral infection can comprise administering an effective amount of a polypeptide construct of the present disclosure to a subject in need thereof, thereby treating the viral infection in the subject. The subject can be a mammal such as a rodent, a non-human primate, or a human. In various cases, the subject treated using a method disclosed herein has been diagnosed with a viral infection (e.g., an infection caused by a virus of the SARS-CoV family). In other instances, the subject treated using a method disclosed herein can be at risk of developing the viral infection, in which case administration of a polypeptide construct of this disclosure to the subject can be considered a prophylactic measure for reducing the risk of developing an infection. In various embodiments, the viral infection is caused by a SARS-CoV-2 variant, e.g., an alpha variant, delta variant, omicron variant, or another variant and/or subvariants (e.g., omega subvariants BA.3, BA.4, BA.5, etc.).

In some embodiments, provided herein is a method of blocking the interaction of a cell surface-bound ACE2 protein and a spike protein of a viral particle, the method comprising contacting a mixture comprising a cell comprising surface-bound ACE2 proteins (e.g., a tracheal or bronchial epithelial cell) and viral particles comprising surface spike proteins (e.g., SARS-CoV-2 virus particles) with an effective amount of a polypeptide construct described herein, thereby intercepting and blocking the interaction between the cell surface-bound ACE2 protein and the spike protein of the viral particle in the mixture. In some cases, the viral particle is a SARS associated viral particle, such as a SARS-CoV or SARS-CoV-2 viral particle. In various cases, the polypeptide construct has an EC₅₀ value for blocking the interaction of the cell surface-bound ACE2 protein and the spike protein of the viral particle in the mixture of ≤10⁻⁹ M, wherein the viral particle is selected from any one of the following SARS-CoV-2 variants: WT, B.1.1.7, B.1.351, B.1.617.2, B.1.1.529 and P.1. In such instances, the polypeptide construct has an EC₅₀ value for blocking the interaction of the cell surface-bound ACE2 protein and the spike protein of the viral particle in the mixture of ≤5×10⁻¹⁰ M, ≤10⁻¹⁰ M, or ≤10⁻¹¹ M.

In various instances, administering a polypeptide construct to the subject, as part of any of the methods described herein, can comprise one or more of parenteral, oral, intranasal, or pulmonary administration. Administering the polypeptide construct to the subject can also comprise using a medical device that allows for mouth inhalation, nasal inhalation, intranasal or pulmonary administration. In some cases, the medical device is a nebulizer. In various embodiments, a method herein comprises intranasal administration of the polypeptide construct to the subject.

As described herein, in various instances, a polypeptide construct can be administered either (i) therapeutically to a subject that has previously been diagnosed with an infection caused by a virus, e.g., a SARS associated virus, or (ii) prophylactically to a subject that may not have been diagnosed with a viral infection but may be considered at risk of becoming infected with the virus, e.g., SARS associated virus, without intervention. In some cases, a method herein comprises treating a disease in a subject caused by an infection with a SARS associated virus, such disease can be selected from SARS-CoV-2 infection is Coronavirus Disease 2019 (COVID-19), Multisystem inflammatory syndrome in adults (MIS-A) and Multisystem inflammatory syndrome in children (MIS-C). In various cases, the subject is a human, a non-human primate, or a rodent.

In various cases, therapeutic effectiveness of a polypeptide construct herein can be assessed in vivo using various direct and/or indirect biomarkers, such as body weight and viral loads of certain organ or cell populations (e.g., the amount of viral RNA detected at such locations) as further described herein.

### VI. Sequences and Sequence Identity

As described in this disclosure, certain embodiments herein relate to an isolated polypeptide or a set of isolated polypeptides forming a polypeptide construct, as well as to a polynucleotide or a set of polynucleotides encoding a polypeptide construct described herein. A polynucleotide in this context can encode all (e.g., all polypeptide chains) or part of a polypeptide construct.

The terms “nucleic acid,” “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers.

A polynucleotide that “encodes” a given polypeptide is a polynucleotide that is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A transcription termination sequence can be located 3′ to the coding sequence.

In certain embodiments, the present disclosure relates to polynucleotide and/or polypeptide sequences that are identical or substantially identical to another polynucleotide and/or polypeptide sequence. The term “identical,” in the context of two or more polynucleotide or polypeptide sequences, refers to two or more sequences or subsequences that are the same, i.e., have the identical sequence of nucleotide or amino acid monomers (i.e., 100% sequence identity), respectively. Polypeptide or polynucleotide sequences herein share “sequence identity” if they have a percentage or a certain number of amino acid residues or nucleotides, respectively, that are at least about 80%, about 85%, about 90%, about 95%, about 97%, or at least about 99% identity over a specified region when compared and aligned for maximum correspondence over a comparison window or over a designated region as measured using one of the commonly used sequence comparison algorithms as known to persons of ordinary skill in the art or by manual alignment and visual inspection. This definition also refers to the complement of a test polynucleotide sequence. The identity can exist over a region that is at least about 50 amino acids or nucleotides in length, or over a region that is from about 75 to about 100 amino acids or nucleotides in length, or, where not specified, across the entire sequence of a polypeptide or polynucleotide. For sequence comparison, typically test sequences are compared to a designated reference sequence. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent (%) sequence identities for the test sequences relative to the reference sequence, based on the program parameters.

The term “comparison window,” as used herein, refers to a segment of a sequence comprising contiguous amino acid or nucleotide positions which can be from about 20 to about 1000 contiguous amino acid or nucleotide positions, for example from about 50 to about 600 or from about 100 to about 300 or from about 150 to about 200 contiguous amino acid or nucleotide positions over which a test sequence can be compared to a reference sequence of the same number of contiguous positions after the two sequences are optimally aligned. Longer segments up to and including the full-length sequence may also be used as a comparison window in certain embodiments. Methods of alignment of sequences for comparison are known to those of ordinary skill in the art. Optimal alignment of sequences for comparison can be conducted, for example, by the local homology algorithm of Smith & Waterman, 1970, Adv. Appl. Math., 2:482c; by the homology alignment algorithm of Needleman & Wunsch, 1970, J. Mol. Biol., 48:443; by the search for similarity method of Pearson & Lipman, 1988, Proc. Natl. Acad. Sci. USA, 85:2444, or by computerized implementations of these algorithms (for example, GAP, BESTFIT, FASTA or TFASTA in the Wisconsin Genetics Software Package, Genetics Computer Group, Madison, WI), or by manual alignment and visual inspection (see, for example, Ausubel et al., Current Protocols in Molecular Biology, (1995 supplement), Cold Spring Harbor Laboratory Press). Examples of available algorithms suitable for determining percent sequence identity are the BLAST and BLAST 2.0 algorithms, which are described in Altschul et al., 1997, Nuc. Acids Res., 25:3389-3402, and Altschul et al., 1990, J. Mol. Biol., 215:403-410, respectively. Software for performing BLAST analyses is publicly available through the website for the National Center for Biotechnology Information (NCBI).

Certain embodiments described herein relate to variant sequences (e.g., variant ACE2 sequences) that comprise one or more amino acid modification, e.g., one or more amino acid insertions, one or more amino acid deletions, and/or one or more amino acid substitutions, when compared to, e.g., a reference such as a wildtype sequence. In certain embodiments, the one or more amino acid modification of a variant sequence comprises one or more amino acid substitutions when compared to a reference such as a wildtype sequence. In such embodiments, the one or more amino acid substitutions are one or more non-conservative substitutions. In other embodiments, the one or more amino acid substitutions are one or more conservative substitutions. In general, a “conservative substitution,” as used herein, is considered to be a substitution of one amino acid with another amino acid having similar physical, chemical and/or structural properties. Common conservative substitutions are listed under Column 1 of TABLE A. One skilled in the art will appreciate that the main factors in determining what constitutes a conservative substitution are usually the size of the amino acid side chain and its physical/chemical properties, but that certain environments allow for substitution of a given amino acid with a broader range of amino acids than those listed in Column 1 of TABLE A. These additional amino acids tend to either have similar properties to the amino acid being substituted but to vary more widely in size or be of similar size but vary more widely in physical/chemical properties. This broader range of conservative substitutions is listed under Column 2 of TABLE A. The skilled person can readily ascertain the most appropriate group of substituents to select from in view of the particular protein environment in which the amino acid substitution is being made.

TABLE A
Conservative Amino Acid Substitutions

| Original Amino | | |
|---|---|---|
| Acid | Column 1 | Column 2 |
| Ala (A) | Gly, Ile, Leu, Met, Norleucine, | Cys, Gly, Ile, Leu, Met, Norleucine, |
| | Val | Phe, Trp, Tyr, Val |
| Arg (R) | His, Lys | His, Lys |
| Asn (N) | Cys, Gln, Ser, Thr | Asp, Cys, Gln, Glu, Ser, Thr |
| Asp (D) | Glu | Asn, Cys, Gln, Glu, Ser, Thr |
| Cys (C) | Asn, Gln, Ser, Thr | Asn, Asp, Gln, Glu, Ser, Thr |
| Gln (Q) | Asn, Cys, Ser, Thr | Asn, Asp, Cys, Glu, Ser, Thr |
| Glu (E) | Asp | Asp, Asn, Cys, Gln, Ser, Thr |
| Gly (G) | Pro | Ala, Ile, Leu, Met, Norleucine, Pro, Val |
| His (H) | Arg, Lys | Arg, Lys, Phe, Trp, Tyr |
| Ile (I) | Ala, Gly, Leu, Met, | Ala, Cys, Gly, Leu, Met, Norleucine, |
| | Norleucine, Val | Phe, Trp, Tyr, Val |
| Leu (L) | Ala, Gly, Ile, Met, Norleucine, | Ala, Cys, Gly, Ile, Met, Norleucine, |
| | Val | Phe, Trp, Tyr, Val |
| Lys (K) | Arg, His | Arg, His |
| Met (M) | Ala, Gly, Ile, Leu, Norleucine, | Ala, Cys, Gly, Ile, Leu, Norleucine, Phe, |
| | Val | Trp, Tyr, Val |
| Phe (F) | Tyr, Trp | Ala, Cys, Gly, His, Ile, Leu, Met, |
| | | Norleucine, Trp, Tyr, Val |
| Pro (P) | Gly | Gly |
| Ser (S) | Asn, Cys, Gln, Thr | Asp, Asn, Cys, Gln, Glu, Thr |
| Thr (T) | Asn, Cys, Gln, Ser | Asp, Asn, Cys, Gln, Glu, Ser |
| Trp (W) | Phe, Tyr | Ala, Cys, Gly, His, Ile, Leu, Met, |
| | | Norleucine, Phe, Tyr, Val |
| Tyr (Y) | Phe, Trp | Ala, Cys, Gly, His, Ile, Leu, Met, |
| | | Norleucine, Phe, Trp, Val |
| Val (V) | Ala, Gly, Ile, Leu, Met, | Ala, Cys, Gly, Ile, Leu, Met, |
| | Norleucine | Norleucine, Phe, Trp, Tyr |

### VII. Additional Embodiments of the Disclosure

Provided herein are polypeptide constructs, pharmaceutical compositions comprising such constructs, as well as methods for producing and using the constructs, e.g., for the treatment of viral infections. Additional embodiments of the disclosure can be according to any one of embodiments 1-31, or combinations thereof.

Embodiment 1. A polypeptide construct comprising (i) an immunoglobin Fc domain (or “Fc domain”) or an Fc scaffold, and (ii) at least three binding domains, wherein each binding domain comprises an angiotensin-converting enzyme 2 (ACE2) protein, wherein the ACE2 protein is capable of binding to the receptor binding domain (RBD) of a spike protein of a severe acute respiratory syndrome (SARS) associated virus, and wherein the at least three binding domains are coupled to the Fc domain.

Embodiment 2. The polypeptide construct of embodiment 1, comprising four, five, ten, or twelve binding domains, wherein each binding domain comprises an ACE2 protein and wherein the ACE2 protein is capable of binding to the RBD of the spike protein of the SARS associated virus.

Embodiment 3. The polypeptide construct of embodiment 1 or 2, wherein the Fc domain is an IgG, IgA, or IgM Fc domain.

Embodiment 4. The polypeptide construct of embodiment 2 or 3, wherein the Fc domain is a trimeric, tetrameric, pentameric or hexameric Fc scaffold.

Embodiment 5. The polypeptide construct of embodiment 4, wherein the Fc domain is a pentameric or hexameric Fc scaffold.

Embodiment 6. The polypeptide construct of embodiment 5, wherein the Fc scaffold is a pentameric IgM Fc scaffold and is coupled to five, six, seven, eight, nine, or ten binding domains, wherein each binding domain comprises an ACE2 protein capable of binding to the spike protein of the SARS-associated virus.

Embodiment 7. The polypeptide construct of embodiment 6, wherein the pentameric IgM Fc scaffold comprises five dimeric Fc domains, each dimeric Fc domain comprising a first Fc polypeptide and a second Fc polypeptide, and each Fc polypeptide comprising a CH2, CH3 and CH4 domain having amino acid sequences having at least about 90%, 95%, 97%, or 99% sequence identity to the sequences set forth in SEQ ID NOs: 5-7, respectively, and wherein the CH4 domain can also comprise an amino acid sequences having at least about 90%, 95%, 97%, or 99% sequence identity to the sequence set forth in SEQ ID NO: 8, instead of SEQ ID NO: 7.

Embodiment 8. The polypeptide construct of any one of embodiments 1-7, wherein each binding domain comprises the extracellular domain of the ACE2 protein.

Embodiment 9. The polypeptide construct of any one of embodiments 1-8, wherein the ACE2 protein of each binding domain comprises or consists of (i) the wild-type ACE2 extracellular domain protein amino acid sequence set forth in SEQ ID NO: 1, (ii) a fragment of the amino acid sequence set forth in SEQ ID NO: 1, or (iii) a sequence having at least about 90%, 95%, 97%, or 99% sequence identity to the sequence set forth in SEQ ID NO: 1.

Embodiment 10. The polypeptide construct of embodiment 9, wherein the fragment of the amino acid sequence set forth in SEQ ID NO: 1 comprises or consists of amino acid residues Q18-S740 (SEQ ID NO: 2) or Q18-A614 (SEQ ID NO: 3) of SEQ ID NO: 1.

Embodiment 11. The polypeptide construct of embodiment 9, wherein the ACE2 protein is a modified ACE2 protein comprising one or more amino acid substitutions relative to the ACE2 wildtype sequences set forth in any one of SEQ ID NOs: 1-3, and wherein such one or more amino acid substitutions are (i) not located in the region of the ACE2 extracellular domain binding domain contacting the spike protein binding domain, and (ii) increase the stability of the binding domain relative to a binding domain comprising an ACE2 protein without the one or more amino acid substitutions, and wherein the increase in stability is measured as an increase in melting temperature of at least about 1° C., 2° C., 3° C., 4° C., 5° C., 6° C., 7° C., or about 8° C. as measured, e.g., using differential scanning calorimetry (DSC). Such one or more amino acid substitutions that can increase the stability (e.g., thermal stability) of an ACE2 protein and thereby enhance the developability of a polypeptide construct can be at any one or more of the positions 73, 180, 226, 293, 304, 311, 342, 360, 362, 372, 373, 396, 411, 412, 417, 423, 447, 453, 507, 520, 522, 526, 533, 550, 569, 647, 650, 686 and 707, based on the sequence set forth in SEQ ID NO: 1. In some embodiments, such one or more substitutions may introduce cysteine residue to form additional disulfide bonds. Such substitutions can include any one or more of A396C_596C, H373C_A412C, A533C_A550C, A311C_A372C, A304C_T362C_A650C_S707C and V293C_L423C. ACE2 proteins comprising such substitutions can comprise or consist of any one of the sequences set forth in SEQ ID NOs: 44-50. In other embodiments, a modified ACE2 protein can have an increased stability by containing one or more amino acid substitutions that increase non-covalent interactions. Such substitutions can be selected from any one or more of L520I, L73Y, A342V, V447I, V226I, Q526F, Q526Y, H417F, T362I, T453L, V647I, S507L, S411H, M360W, M360F, Q522M, Y180W, S411I, Q526H, T686I and V647L. ACE2 proteins comprising such substitutions can comprise or consist of any one of the sequences set forth in SEQ ID NOs: 51-72.

Embodiment 12. The polypeptide construct of embodiment 11, wherein the modified ACE2 protein comprises or consists of the amino acid sequence set forth in SEQ ID NO: 4.

Embodiment 13. The polypeptide construct of embodiment 5 or 6, comprising a pentameric IgM Fc scaffold coupled to ten binding domains, wherein each first and second Fc polypeptide of the five dimeric Fc domains is coupled to one of the ten binding domains.

Embodiment 14. The polypeptide construct of embodiment 13, wherein each binding domain is coupled to an Fc polypeptide either directly or via a peptide linker.

Embodiment 15. The polypeptide construct of any one of embodiments 4-14, wherein the Fc scaffold further comprises a tailpiece assembly, wherein the C-terminal tailpiece sequences (e.g., SEQ ID NO: 9) of the Fc polypeptides form the tailpiece assembly.

Embodiment 16. The polypeptide construct of embodiment 15, wherein the polypeptide construct further comprises a J-chain.

Embodiment 17. The polypeptide construct of embodiment 14, wherein the peptide linker comprises up to about 50 amino acids.

Embodiment 18. The polypeptide construct of embodiment 13, comprising: (i) a pentameric IgM Fc scaffold comprising five dimeric Fc domains, each comprising a first and a second Fc polypeptide, and each Fc polypeptide comprising a tailpiece sequence, (ii) ten binding domains each comprising an ACE2 protein comprising an amino acid sequence having at least about 90%, 95%, 97%, 99%, or 100% sequence identity to any of the sequences set forth in SEQ ID NOs: 1-4, wherein each first and second Fc polypeptide of the five dimeric Fc domains is coupled via its N-terminus to one of the ten binding domains, and (iii) a tailpiece assembly, wherein the C-terminal tailpiece sequences of the Fc polypeptides form the tailpiece assembly.

Embodiment 19. The polypeptide construct of embodiment 18, wherein the polypeptide construct is the construct with variant number v36708 or v36709.

Embodiment 20. The polypeptide construct of any one of embodiments 1-19, wherein the SARS associated virus is SARS-coronavirus (SARS-CoV) or SARS-coronavirus 2 (SARS-CoV-2).

Embodiment 21. The polypeptide construct of embodiment 20, wherein the SARS-CoV-2 is a variant, wherein the variant is any one of alpha, beta, gamma, delta, omicron, mu, eta, iota, kappa, lambda, epsilon, theta, or zeta variant, or any variant having a spike protein capable of binding to an ACE2 protein of the polypeptide construct.

Embodiment 22. A pharmaceutical composition comprising the polypeptide construct of any one of embodiments 1-21, and a pharmaceutically acceptable carrier.

Embodiment 23. A nucleic acid molecule or a set of nucleic acid molecules encoding the polypeptide construct of any one of embodiments 1-21.

Embodiment 24. A host cell comprising the nucleic acid molecule or the set of nucleic acid molecules according to embodiment 23.

Embodiment 25. A method of producing the polypeptide construct of any one of embodiments 1 to 21, the method comprising (i) expressing the polypeptide construct in a host cell of a host cell culture, the host cell comprising a nucleic acid molecule or a set of nucleic acid molecules encoding the polypeptide construct, and (ii) recovering the polypeptide construct from the host cell culture.

Embodiment 26. A method of administering to a subject in need thereof an effective amount of a polypeptide construct of any one of embodiments 1-21.

Embodiment 27. The method of embodiment 26, wherein administering the polypeptide construct comprises parenteral, oral, mouth inhalation, nasal inhalation, intranasal or pulmonary administration.

Embodiment 28. The method of embodiment 26 or 27, wherein administering the polypeptide construct comprises using a medical device that allows for mouth inhalation, nasal inhalation, intranasal or pulmonary administration.

Embodiment 29. The method of any one of embodiments 26-28, wherein the polypeptide construct is administered to the subject either (i) therapeutically following a diagnosis of a viral infection caused by a SARS associated virus, or (ii) prophylactically as the subject is considered of being at risk of becoming infected with the SARS associated virus.

Embodiment 30. The method of embodiment 29, wherein the SARS associated virus is a SARS-CoV or SARS-CoV-2 associated virus, and causes a disease in the subject selected from SARS-CoV-2 infection is Coronavirus Disease 2019 (COVID-19), Multisystem inflammatory syndrome in adults (MIS-A) and Multisystem inflammatory syndrome in children (MIS-C).

Embodiment 31. The method of any one of embodiments 26-30, wherein the subject is a human, a non-human primate, or a rodent.

Embodiment 32. A polypeptide construct, comprising: (i) at least three binding domains, each binding domain comprising an ACE2 protein capable of binding to a viral spike protein; (ii) at least three dimeric Fc domains, each dimeric Fc domain comprising a first Fc polypeptide and a second Fc polypeptide, the first and the second Fc polypeptide each comprising a tailpiece sequence; and (iii) a tailpiece assembly, wherein: a) at least one of the first and the second Fc polypeptide of each dimeric Fc domain is coupled via its N-terminus to one of the at least three binding domains, and b) the tailpiece sequences of the first and second Fc polypeptides form the tailpiece assembly.

Embodiment 33. The polypeptide construct of embodiment 32, comprising at least four, five, or six binding domains, wherein each binding domain is coupled to the N-terminus of a first or second Fc polypeptide.

Embodiment 34. The polypeptide construct of any one of embodiments 32-33, comprising six binding domains, each binding domain coupled to the N-terminus of a first or second Fc polypeptide, thereby forming a hexavalent polypeptide construct.

Embodiment 35. The polypeptide construct of any one of embodiments 32-34, comprising four, five or six dimeric Fc domains.

Embodiment 36. The polypeptide construct of embodiment 35, comprising five dimeric Fc domains.

Embodiment 37. The polypeptide construct of any one of embodiments 35-36, comprising five dimeric Fe domains and ten binding domains, wherein each binding domain is coupled to the N-terminus of a first or second Fc polypeptide of the five dimeric Fc domains, thereby forming a decavalent polypeptide construct.

Embodiment 38. The polypeptide construct of embodiment 35, comprising six dimeric Fc domains.

Embodiment 39. The polypeptide construct of embodiment 38, comprising twelve binding domains, wherein each binding domain is coupled to the N-terminus of a first or second Fc polypeptide of the six dimeric Fc domains, thereby forming a dodecavalent polypeptide construct.

Embodiment 40. A polypeptide construct, comprising: (i) ten binding domains, each binding domain comprising an ACE2 protein capable of binding to a viral spike protein; (ii) five dimeric Fc domains, each dimeric Fc domain comprising a first Fc polypeptide and a second Fc polypeptide, the first and the second Fc polypeptide each comprising a tailpiece sequence; and (iii) a tailpiece assembly, wherein: a) each of the ten Fc polypeptides is coupled via its N-terminus to one of the ten binding domains, and b) the tailpiece sequences of the first and second Fc polypeptides form the tailpiece assembly.

Embodiment 41. The polypeptide construct of any one of embodiments 32-40 wherein at least one of the binding domains comprises an ACE2 protein comprising or consisting of an amino acid sequence having at least about 80%, 90%, 95%, 97%, 99%, or 100% sequence identity to any of the amino acid sequences set forth in SEQ ID NOs: 1-3.

Embodiment 42. The polypeptide construct of any one of embodiments 32-41, wherein each binding domain comprises an ACE2 protein comprising or consisting of an amino acid sequence having at least about 80%, 90%, 95%, 97%, 99%, or 100% sequence identity to any of the amino acid sequences set forth in SEQ ID NOs: 1-3.

Embodiment 43. The polypeptide construct of any one of embodiments 32-42, wherein each binding domain comprises or consists of an ACE protein comprising or consisting of any one of the amino acid sequences set forth in SEQ ID NOs: 1-3.

Embodiment 44. The polypeptide construct of any one of embodiments 32-43, wherein at least one of the binding domains comprises an ACE2 protein comprising one or more amino acid substitutions relative to any of the amino acid sequences set forth in SEQ ID NOs: 1-3.

Embodiment 45. The polypeptide construct of embodiment 44, wherein the one or more amino acid substitutions are located outside the region interacting with the binding domain of the viral spike protein.

Embodiment 46. The polypeptide construct of any one of embodiments 44-45, wherein the one or more amino acid substitutions comprise the catalytic knock-out (KO) mutations H374N_H378N relative to the sequence set forth in SEQ ID NO: 1, resulting in a catalytic KO sequence of the extracellular domain of ACE2 set forth in SEQ ID NO: 4.

Embodiment 47. The polypeptide construct of any one of embodiments 44-46, wherein the one or more amino acid substitutions comprise substitution(s) that increase the stability of the binding domain relative to a binding domain comprising an ACE2 protein without the one or more amino acid substitutions, and wherein the increase in stability is measured as an increase in melting temperature of at least about 1° C., 2° C., 3° C., 4° C., 5° C., 6° C., 7° C., or about 8° C. as measured using differential scanning calorimetry (DSC).

Embodiment 48. The polypeptide construct of any one of embodiments 32-46, wherein at least one of the dimeric Fc domains is an IgG or an IgA Fc domain.

Embodiment 49. The polypeptide construct of embodiment 48, wherein all of the dimeric Fc domains are either IgG Fc domains or IgA Fc domains.

Embodiment 50. The polypeptide construct of any one of embodiments 32-49, wherein all of the dimeric Fc domains are IgM Fc domains, thereby forming a multimeric IgM Fc scaffold.

Embodiment 51. The polypeptide construct of any one of embodiments 32-50, wherein each first and second Fc polypeptide of each dimeric Fc domain comprises a CH2 domain, a CH3 domain, a CH4 domain, or a combination thereof.

Embodiment 52. The polypeptide construct of embodiment 51, wherein polypeptide construct comprises a pentameric IgM Fc scaffold comprising five dimeric Fc domains, each comprising a first and a second Fc polypeptide, and wherein each first and second Fc polypeptide comprises a CH2 domain, a CH3 domain and a CH4 domain.

Embodiment 53. The polypeptide construct of embodiment 52, wherein (i) the CH2 domain comprises or consists of an amino acid sequence having at least about 80%, 90%, 95%, 97%, 99%, or 100% sequence identity to the amino acid sequence set forth in SEQ ID NO: 5, (ii) the CH3 domain comprises or consists of an amino acid sequence having at least about 80%, 90%, 95%, 97%, 99%, or 100% sequence identity to the amino acid sequence set forth in SEQ ID NO: 6, and/or (iii) the CH4 domain comprises or consists of an amino acid sequence having at least about 80%, 90%, 95%, 97%, 99%, or 100% sequence identity to the amino acid sequence set forth in SEQ ID NO: 7.

Embodiment 54. The polypeptide construct of any one of embodiments 32-53, wherein each binding domain is coupled to the N-terminus of a first or a second Fc polypeptide directly and without a linker.

Embodiment 55. The polypeptide construct of any one of embodiments 32-53, wherein (i) at least one binding domain is coupled to the N-terminus of a first or a second Fc polypeptide via a first linker, or (ii) all binding domains are each coupled to the N-terminus of a first or a second Fc polypeptide via a first linker.

Embodiment 56. The polypeptide construct of any one of embodiments 32-55, wherein the tailpiece sequences of the first and second Fc polypeptides form the tailpiece assembly through covalent interactions, non-covalent interactions, or a combination thereof.

Embodiment 57. The polypeptide construct of embodiment 56, wherein the second linker comprises or consists of an amino acid sequence having at least about 80%, 90%, or 100% sequence identity to the amino acid sequence set forth in SEQ ID NO: 9.

Embodiment 58. The polypeptide construct of any one of embodiments 32-57, further comprising a J-chain.

Embodiment 59. The polypeptide construct of embodiment 58, wherein the J-chain comprises or consists of an amino acid sequence having at least about 80%, 90%, or 100% sequence identity to the amino acid sequence set forth in SEQ ID NO: 10 or SEQ ID NO: 12.

Embodiment 60. The polypeptide construct of any one of embodiments 50-59, wherein the polypeptide construct comprises: (i) ten binding domains, each binding domain comprising or consisting of an ACE2 protein capable of binding to a viral spike protein, and comprising or consisting of an amino acid sequence having at least about 80%, 90%, 95%, 97%, 99%, or 100% sequence identity to any of the amino acid sequences set forth in SEQ ID NOs: 2-4; (ii) a pentameric IgM scaffold comprising five dimeric Fc domains, each dimeric Fc domain comprising a first Fc polypeptide and a second Fc polypeptide, the first and the second Fc polypeptide each comprising a tailpiece sequence; a tailpiece assembly; and a J-chain, wherein: a) each of the ten Fc polypeptides is coupled via its N-terminus to one of the ten binding domains, thereby forming ten Fc-binding domain fusion polypeptides, and b) the tailpiece sequences of the first and second Fc polypeptides form the tailpiece assembly.

Embodiment 61. The polypeptide construct of embodiment 60, wherein at least one of the ten Fc-binding domain fusion polypeptides comprises or consists of an amino acid sequence having at least about 80%, 90%, 95%, 97%, 99%, or 100% sequence identity to the amino acid sequence set forth in SEQ ID NO: 28 or SEQ ID NO: 30.

Embodiment 62. The polypeptide construct of any one of embodiments 60-61, wherein polypeptide construct is v36708 or v36709.

Embodiment 63. The polypeptide construct of any one of embodiments 32-62, wherein the viral spike protein is derived from a SARS associated virus.

Embodiment 64. The polypeptide construct of embodiment 63, wherein the SARS associated virus is SARS-coronavirus (SARS-CoV) or SARS-coronavirus 2 (SARS-CoV-2).

Embodiment 65. The polypeptide construct of any one of embodiments 32-64, wherein the polypeptide construct has an apparent K_{D} for binding a viral spike protein of ≤10⁻⁹ M or ≤10⁻¹⁰ M.

Embodiment 66. The polypeptide construct of any one of embodiments 32-65, wherein the polypeptide construct has an EC₅₀ value for neutralizing the interaction of cell surface-bound ACE2 and a spike protein of a SARS-CoV-2 viral particle of ≤5×10⁻¹⁰ M, wherein the SARS-CoV-2 viral particle is selected from any one of the following SARS-CoV-2 variants: WT, B.1.1.7, B.1.351 and P.1.

Embodiment 67. The polypeptide construct of any one of embodiments 32-66, wherein the polypeptide construct has an EC₅₀ value, given as %-inhibition, for neutralizing the interaction of cell surface-bound ACE2 and a spike protein of a SARS-CoV-2 pseudotyped lentivirus of ≤10⁻¹¹ M or ≤10⁻¹² M.

Embodiment 68. A pharmaceutical composition comprising the polypeptide construct of any one of embodiments 32-67, and a pharmaceutically acceptable carrier or excipient.

Embodiment 69. The pharmaceutical composition of embodiment 68, formulated for nasal or oral administration.

Embodiment 70. A nucleic acid molecule or a set of nucleic acid molecules encoding the polypeptide construct of any one of embodiments 32-69.

Embodiment 71. A host cell comprising the nucleic acid molecule or the set of nucleic acid molecules according to embodiment 70.

Embodiment 72. A method of producing the polypeptide construct of any one of embodiments 32-69, the method comprising: (a) expressing the polypeptide construct in a host cell of a host cell culture, the host cell comprising a nucleic acid molecule or a set of nucleic acid molecules encoding the polypeptide construct, and (b) recovering the polypeptide construct from the host cell culture.

Embodiment 73. The method of embodiment 72, further comprising, following step (b), purifying the polypeptide construct such that the polypeptide construct has a chemical purity of ≥95%.

Embodiment 74. A method of blocking the interaction of a cell surface-bound ACE2 protein and a spike protein of a viral particle, the method comprising contacting a mixture comprising a cell comprising surface-bound ACE2 proteins and viral particles comprising surface spike proteins with an effective amount of a polypeptide construct of any one of embodiments 32-69, thereby blocking the interaction of the cell surface-bound ACE2 protein and the spike protein of the viral particle in the mixture.

Embodiment 75. The method of embodiment 74, wherein the viral particle is a SARS associated viral particle, such as a SARS-CoV or SARS-CoV-2 viral particle.

Embodiment 76. The method of any one of embodiments 74-75, wherein the polypeptide construct has an EC₅₀ value for blocking the interaction of the cell surface-bound ACE2 protein and the spike protein of the viral particle in the mixture of ≤5×10⁻¹⁰ M, wherein the viral particle is selected from any one of the following SARS-CoV-2 variants: WT, B.1.1.7, B.1.351, P.1, B.1.617.2 and B.1.1.529.

Embodiment 77. A method of treating a viral infection in a subject in need thereof, the method comprising administering to the subject an effective amount of a polypeptide construct of any one of embodiments 32-69, thereby treating the viral infection in the subject.

Embodiment 78. The method of embodiment 77, wherein administering the polypeptide construct to the subject comprises parenteral, oral, intranasal, or pulmonary administration.

Embodiment 79. The method of any one of embodiments 77-78, wherein administering the polypeptide construct to the subject comprises using a medical device that allows for mouth inhalation, nasal inhalation, intranasal or pulmonary administration.

Embodiment 80. The method of embodiment 79, wherein the medical device is a nebulizer.

Embodiment 81. The method of any one of embodiments 77-80, wherein the viral infection is caused by a SARS associated virus, optionally a SARS-CoV associated virus or a SARS-CoV-2 associated virus.

Embodiment 82. The method of embodiment 81, wherein the SARS associated virus causes a disease in the subject selected from SARS-CoV-2 infection is Coronavirus Disease 2019 (COVID-19), Multisystem inflammatory syndrome in adults (MIS-A) and Multisystem inflammatory syndrome in children (MIS-C).

Embodiment 83. The method of any one of embodiments 77-82, wherein the subject is a human, a non-human primate, or a rodent.

Embodiment 84. A polypeptide construct of any one of embodiments 32-69 for use in the treatment of a viral infection.

Embodiment 85. Use of a polypeptide construct of any one of embodiments 32-69 in the manufacture of a medicament for the treatment of a viral infection.

### EXAMPLES

The following Examples are provided for illustrative purposes and are not intended to limit the scope of the invention in any way. The reagents employed in the examples are commercially available or can be prepared using commercially available instrumentation, methods, or reagents known in the art, unless specified otherwise. The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the appended claims.

### Example 1

### Design and Production of Multivalent ACE2 Decoy Polypeptide Constructs

Multivalent ACE2 decoy polypeptide constructs were designed based on IgG, IgA, and/or IgM domains and scaffold, respectively, expressed in transient mammalian cell cultures, and purified as described herein.

### Methods

### Design of ACE2 Decoy Polypeptide Constructs

Schematic representations of the spike protein-binding polypeptide constructs according to certain embodiments of this disclosure are illustrated in FIGS. 1A-1M. The constructs were designed using IgG Fc, IgA Fc, or IgM Fc domains or scaffolds, respectively, to achieve different valency of ACE2 per construct. The extracellular domain of the ACE2 protein spanning residues Q18-S740 (SEQ ID NO: 2) or Q18-615 (SEQ ID NO: 3) of human wildtype ACE2 (accession number: Q9BYF1, SEQ ID NO: 1), or an ACE2 protein containing the catalytic knockout (KO) mutations H374N_H378N compared to wild type human ACE2 (residue numbering based on SEQ ID NO: 1), resulting in a KO polypeptide with SEQ ID NO: 4 (see, e.g., Moore, M. J. et al. Retroviruses pseudotyped with the severe acute respiratory syndrome coronavirus spike protein efficiently infect cells expressing angiotensin-converting enzyme 2. J Virol 78, 10628-10635, 2004), was used for the construction of the ACE2 decoy polypeptide constructs described herein.

For IgM-based ACE2 decoy polypeptide constructs, IgM Fc polypeptides (containing domains CH2, CH3 and CH4, e.g., SEQ ID NOs: 5-7, respectively) with a tailpiece sequence (KPTLYNVSLVMSDTAGTCY, SEQ ID NO: 9) was used to enable pentameric multimerization of five dimeric Fc domains in the presence of the J-chain (v23742, SEQ ID NO: 12) to give a pentameric IgM Fc scaffold. In addition, either wildtype (wt) ACE2 (wt ACE2, SEQ ID NO: 2) or ACE2 with catalytic knockout mutations (ACE2 catalytic KO, SEQ ID NO: 4) was fused to the N-terminus of the IgM CH2 domains of the IgM Fc scaffold, resulting either in the wt ACE2-IgM Fc construct v26010 (SEQ ID NO: 28) or the ACE2 catalytic KO IgM Fc v26014 (SEQ ID NO: 30), respectively. Co-expression of the ACE2 IgM heavy chain (HC, IgM Fc polypeptides) and J-chain led to covalent assembly of the pentameric IgM-derived construct comprising 5 dimeric IgM Fc domains, each comprising either a total of ten wt ACE2-IgM Fc polypeptides, v26010, or ten ACE2 catalytic KO IgM Fc polypeptides, v26014, respectively, and hence being decavalent for ACE2 (see, TABLE 1, and FIG. 1A illustrating the structure of the resulting polypeptide constructs).

An IgA-based ACE2 decoy polypeptide constructs was designed using IgA1 Fc with an IgA2 hinge region and an intact tailpiece sequence required for multimerization. The IgA2 hinge domain boundaries were used as described previously (see, e.g., Borrok, M. J. et al. Enhancement of antibody-dependent cell-mediated cytotoxicity by endowing IgG with FcαRI (CD89) binding. mAbs 7, 743-751, 2015).

For IgA-based ACE2 decoy constructs, in order to eliminate covalent interaction with the secretory component, a mutation was introduced in the IgA1 CH2 domain (C5092S (C192S) compared to wildtype IgA1 CH2). This cysteine in wildtype IgA1 can form a disulfide bond with the secretory component. Wt ACE2 (SEQ ID NO: 2) or ACE2 with catalytic KO (SEQ ID NO: 4) was fused to the N-terminus of the IgA2 hinge-IgA1 Fc chimera resulting in wt ACE2-IgA Fc v26009 or ACE2 catalytic KO-IgA Fc v26013, respectively, and co-expressed with J-chain (v23742, SEQ ID NO: 12) to attain dimeric IgA molecule that is tetravalent for ACE2 (see, TABLE 1, and FIGS. 1D-F illustrating the structure of the resulting polypeptide constructs).

In addition, IgG-based ACE2 decoy polypeptide constructs with any one of monovalent, bivalent, or tetravalent ACE2 were also designed and produced herein. In these variants, ACE2 was fused either (i) directly to the N-terminus of the IgG1 hinge resulting in wt ACE2 N-term IgG Fc v26008 or v27618, or ACE2 catalytic KO N-term IgG Fc v26012 or v27620, (ii) to the C-terminus of the IgG Fc using a (G₄S)₃ linker resulting in constructs ACE2 catalytic KO C-term IgG Fc v27656 or v27657), or (iii) to both the IgG1 N- and C-termini to yield the construct ACE2 catalytic KO N- and C-term IgG Fc v27621), obtaining IgG-based constructs with either one, two, or four ACE2 domains attached, respectively (see, e.g., TABLE 1, and FIGS. 1G-M illustrating the structure of the resulting polypeptide constructs).

For certain polypeptide constructs, and to allow for selective heterodimeric pairing of Fc polypeptides of monovalent ACE2 IgG decoy variants, mutations were introduced into the CH3 domains of the IgG Fc polypeptides as described previously (see, e.g., Von Kreudenstein, T. S. et al. Improving biophysical properties of a bispecific antibody domain to aid developability: quality by molecular design. mAbs 5, 646-654, doi:10.4161/mabs.25632 (2013)).

Furthermore, the mutations L234A_L235A_D265S—as compared to a wild type human IgG1 CH2 domain—were also introduced in both CH2 domains of the first and second Fc polypeptides to reduce binding to Fe gamma receptors. IgG-based ACE2 decoy construct designs included the following constructs: IgG with two N-terminal ACE2 domains (e.g., construct(s) with wt ACE2, v36710, and those with ACE2 catalytic KO, v36711), IgG with one N-terminal ACE2 domain (e.g., construct(s) with wt ACE2, v36712, and those with ACE2 catalytic KO, v36713), IgG with two C-terminal ACE2 domains (e.g., construct(s) with ACE2 catalytic KO, v36792), IgG with one C-terminal ACE2 domain (e.g., construct(s) with ACE2 catalytic KO v36800), and IgG with two N-terminal and two C-terminal ACE2 domains (e.g., construct(s) with ACE2 catalytic KO v36715) (see, also, TABLE 1).

Some constructs contained a single G₄S linked myc-tag (GGGGSEQKLISEEDL (SEQ ID NO: 73)) for detection purposes by fusion of the myc-tag to the C-terminus of the J-chain (CL_#26045) for IgA ACE2 decoys (wt ACE2 v34797 and ACE2 catalytic KO v34798) and IgM ACE2 decoys (wt ACE2 v34799 and ACE2 catalytic KO v34800) (see, also, TABLE 1). A single myc-tag per IgG variant was achieved using mutations introduced in the CH3 domains of the IgG Fc as described previously (Von Kreudenstein, T. S. et al. Improving biophysical properties of a bispecific antibody domain to aid developability: quality by molecular design. MAbs 5, 646-654, doi:10.4161/mabs.25632 (2013)). IgG with G₄S linked C-terminal myc-tag (wt ACE2 CL_#26007 and ACE2 catalytic KO CL_#26011) was used to generate either myc-tagged IgG with one N-terminal ACE2 (wt ACE2 v34803 and ACE2 catalytic KO v34804) or two N-terminal ACE2 domains (wt ACE2 v34801 and ACE2 catalytic KO v34802) (see, also, TABLE 1).

A hexameric IgM ACE2 decoy polypeptide construct was designed and produced by expressing ACE2 (either wt ACE2 IgM Fc CL_#26010, SEQ ID NO: 2, or ACE2 catalytic KO IgM Fc CL_#26014, SEQ ID NO: 4) fused to an IgM Fc domain polypeptide in the absence of a J-chain to obtain the IgM hexamer that comprised six dimeric Fc domains and was dodecavalent for ACE2 and corresponding spike protein binding.

### Expression and Purification of ACE2 Decoy Polypeptide Constructs

Sequences of ACE2 decoy polypeptide constructs designed as shown in TABLE 1 herein were ported into expression vectors and expressed and purified as follows. ACE2 decoy constructs were produced by transient transfection of expression plasmids into Expi293F™ cells using ExpiFectamine™ 293 Transfection Kit (Thermo Fisher Scientific) as per manufacturer's protocol. The ratio of plasmids used for expression was optimized to produce ACE2 decoy constructs in suitable yield and high purity. Briefly, DNA was diluted in Opti-MEM™ I Reduced Serum Medium, mixed with pre-diluted ExpiFectamine™ reagent for 10-20 minutes at room temperature, and added into Expi293 cells (3×106 cells/mL). 5-7 days post transfection, the supernatants from transfected cells were harvested for purification.

The IgG-based ACE2 decoy constructs were purified using protein A affinity chromatography (mAb Select SuRe, Cytiva Life Sciences) followed by size-exclusion chromatography (SEC) (Superdex 200 Increase 10/300 GL, Cytiva Life Sciences) to remove aggregates. IgA and IgM-based ACE2 decoy polypeptide constructs were purified using anion exchange chromatography (Foresight™ Nuvia™ HP-Q column, Bio-Rad Laboratories), followed by an additional step using ceramic hydroxyapatite mixed-mode chromatography (Foresight™ CHT™ type II column, Bio-Rad Laboratories). The final samples were analyzed on reduced and non-reduced capillary electrophoresis-SDS (LabChip® GXII Touch™, PerkinElmer) or SDS-PAGE and analytical SEC to confirm >95% purity of the produced decoy polypeptide constructs. Purified proteins were quantitated based on A280 measurements (Nanodrop) and stored at −80° C. until further use.

### Certain ACE2-Containing Decoy Polypeptide Constructs Designed and Produced Herein

TABLE 1
Exemplary Mono- and Multivalent ACE2 Decoy Polypeptide Constructs

| Construct | Exemplary | | ACE2 | Chain H1 | Chain H2 | J-Chain |
|---|---|---|---|---|---|---|
| Number | Schematic | Construct Description | valency | variant | variant | variant |
| 34803 | FIG. 1I | ACE2 IgG OAA with myc tag | 1 | 26007 | 12153 | NA |
| 34804 | FIG. 1I | ACE2 catalytic KO IgG OAA | 1 | 26011 | 12153 | NA |
| | | with myc tag | | | | |
| 34801 | FIG. 1H | ACE2 IgG FSA with myc tag | 2 | 26007 | 26008 | NA |
| 34802 | FIG. 1H | ACE2 catalytic KO IgG FSA | 2 | 26011 | 26012 | NA |
| | | with myc tag | | | | |
| 34797 | FIG. 1D | ACE2 IgA dimer with myc tag | 4 | 26009 | NA | 26045 |
| 34798 | FIG. 1D | ACE2 catalytic KO IgA dimer | 4 | 26013 | NA | 26045 |
| | | with myc tag | | | | |
| 34799 | FIG. 1A | ACE2 IgM pentamer | 10 | 26010 | NA | 26045 |
| | | with myc tag | | | | |
| 34800 | FIG. 1A | ACE2 catalytic KO IgM pentamer | 10 | 26014 | NA | 26045 |
| | | with myc tag | | | | |
| 36712 | FIG. 1I | ACE2 IgG OAA | 1 | 26008 | 12155 | NA |
| 36713 | FIG. 1I | ACE2 catalytic KO IgG OAA | 1 | 26012 | 12155 | NA |
| 36710 | FIG. 1H | ACE2 IgG FSA | 2 | 27618 | NA | NA |
| 36711 | FIG. 1H | ACE2 catalytic KO IgG FSA | 2 | 27620 | NA | NA |
| 36706 | FIG. 1D | ACE2 IgA dimer | 4 | 26009 | NA | 23742 |
| 36707 | FIG. 1D | ACE2 catalytic KO IgA dimer | 4 | 26013 | NA | 23742 |
| 36708 | FIG. 1A | ACE2 IgM pentamer | 10 | 26010 | NA | 23742 |
| 36709 | FIG. 1A | ACE2 catalytic KO IgM | 10 | 26014 | NA | 23742 |
| | | pentamer | | | | |
| 36715 | FIG. 1G | ACE2 catalytic KO tetravalent | 4 | 27621 | NA | NA |
| | | IgG | | | | |
| 36792 | FIG. 1L | ACE2 catalytic KO C-terminal | 2 | 27656 | NA | NA |
| | | bivalent IgG | | | | |
| 36800 | FIG. 1M | ACE2 catalytic KO C-terminal | 1 | 27657 | 12155 | NA |
| | | monovalent IgG | | | | |

For further reference, in FIGS. 1A-1M, ACE2 domains are white, IgM CH domains are colored with small, checkered pattern, IgA CH domains are colored with cross-hatch pattern, and IgG CH domains are colored with zig-zag pattern. The intercalated tailpieces of IgA and IgM are represented by a dark grey rectangular stacked assembly, and the J-chain is represented as light grey U-shape. Myc-tag is denoted as black rectangle with “myc” text. FIGS. 1A-1M abbreviations: H1, heavy chain 1; H2, heavy chain 2; J, J-chain; OAA, one armed antibody; FSA, full size antibody; KO, knockout; NA, not applicable.

### Results

ACE2 decoy polypeptide construct of various formats and ACE2 valency were constructed and purified from mammalian cell culture supernatants and determined to have >95% purity by SDS-PAGE and analytical SEC with >1 mg yield.

### Example 2

### Binding of ACE2 Decoy Polypeptide Constructs to SARS-CoV-2 Spike Protein

The ACE2 decoy polypeptide constructs described and produced in EXAMPLE 1 herein and as shown and described in TABLE 1 above, were assessed for binding to SARS-CoV-2 spike protein receptor binding domain (RBD) using surface plasmon resonance (SPR) and flow cytometry.

### Methods

A Biacore T200 instrument (Cytiva/Danaher, Washington D.C., USA, product #28975001) was used to measure the binding kinetics of ACE2 decoy constructs to SARS-CoV-2 spike protein. SPR experiments were conducted at a temperature of 25° C. using HBS EP+(Cytiva/Danaher, Washington D.C., USA), 10 mM HEPES, 150 mM NaCl, 3 mM EDTA, and 0.05% v/v surfactant P20 as running buffer. Series S Sensor chip CM5, (Cytiva/Danaher, Washington D.C., USA, product #29149603), HBS EP+10× buffer (Cytiva/Danaher, Washington D.C., USA, product #BR100669), 10 mM Sodium Acetate pH 5.5 (Cytiva/Danaher, Washington D.C., USA, product #BR100352), NaOH 50 (Cytiva/Danaher, Washington D.C., USA, product #BR100358), 10 mM Glycine-HCL pH3.0 (Cytiva/Danaher, Washington D.C., USA, product #BR100357), amine coupling kit (Cytiva/Danaher, Washington D.C., USA, product #BR100050) were all purchased from Cytiva. SARS-CoV-2 spike RBD-His was purchased from R&D Systems (R&D Systems, Minneapolis, MN, USA, cat #10500-CV).

Binding kinetics of IgG, IgA, and IgM ACE2 decoy constructs for SARS-CoV-2 spike RBD-His were measured under the different ligand densities of SARS-CoV-2 spike RBD-His on the sensor chip. SARS-CoV-2 spike RBD-His was covalently immobilized on the CM5 Series S sensor chip by amine coupling method. To that end, EDC/NHS mix was injected for 7 mins at 10 μl/min for activating the sensor chip surface. Then 1.5, 1.875, 2, and 3 μg/ml SARS-CoV-2 spike RBD-His in 10 mM sodium acetate pH 5.5 was injected on sample flow cells at 10 μl/min to reach surface ligand densities of 7.6, 14.4, 115, and 409 resonance units (RUs), respectively. Buffer was injected on the reference cell. Ethanolamine was introduced for 7 mins at 10 μl/min to deactivate the surface.

IgG, IgA, and IgM ACE2 decoy constructs were then injected on both reference and sample flow cells as analytes. Nine concentrations following a two-fold dilution series of IgG, IgA, and IgM ACE2 decoy constructs were injected at a flow rate of 30 μl/min. The concentration ranges and association/dissociation times used are reported in TABLE 2. 10 mM glycine-HCl pH 3.0 was used to regenerate the surface between each cycle for 30 s at 30 μl/min. The binding kinetics were analyzed using the 1:1 binding model and kinetic analyses were performed using Biacore T200 Evaluation Software v3.0. Concentration ranges and association/dissociation times used in SPR experiments to measure ACE2 decoy construct and SARS-CoV-2 spike RBD protein binding kinetics are shown in TABLE 2:

TABLE 2
Spike Protein Binding Assay Parameters

| | Concentration | | |
|---|---|---|---|
| | range of ACE2 | Association | Disassociation |
| | decoy construct | time | time |
| Construct | tested (nM) | (s) | (s) |
| ACE2 IgG | 1.95-500 | 400 | 600 |
| OAA (v34803) | | | |
| ACE2 IgG | 1.56-200 | 400 | 600 |
| FSA (v34801) | | | |
| ACE2 IgA | 0.78-100 | 400 | 600 |
| dimer (v34797) | | | |
| ACE2 IgM | 0.78-100 | 400 | 600 |
| pentamer (v34799) | | | |

### Generation of Transient Spike Expressing HEK2936e Cells

HEK2936e cells (>90% viable, National Research Council (NRC) License) were transiently transfected with human full length wild type (WT), B.1.1.7 or B.1.351 spike (pCMV3 backbone, Sino Biological, #VG40589-UT, VG40771-UT, VG40772-UT, respectively) or mock GFP negative control (pD2610-CMV(v23)-GFP, in-house preparation) using 293fectin Transfection Reagent (Themo, #12347019).

On the day of transfection, cells were seeded at 1 million/mL in complete growth media (Freestyle 293+1% FBS Gibco, #12338-018 and 12483020, respectively) and incubated at 37° C.+5% CO₂ with shaking at 125 rpm while transfection reagents were prepared according to the manufacturer's protocol.

A combination of DNA (1 μg DNA per 1 million cells) and OptiMEM (Thermo, #31985062) or 293fectin and OptiMEM, were incubated in separate tubes for 5 min at room temperature. Following the incubation, reagents were mixed in equivalent volume and incubated at room temperature for 20 minutes. The DNA:293Fectin mixture was added dropwise to pre-warmed cells and the culture was incubated overnight at 37° C., 5% CO₂ with shaking at 125 rpm.

Following transfection, cells were counted and frozen in 10% DMSO (Sigma Aldrich, #D8418+90% FBS or resuspended in FACS buffer (PBS containing 2% FBS) in preparation for quantitative flow cytometry.

### Assessment of Spike Expression Using Quantitative Flow Cytometry

Quantification of spike protein expression on transfected HEK2936e cells was performed by flow cytometry using Quantum™ Simply Cellular® anti-Human IgG beads (Bangs Laboratories, #816).

Suspension cells were harvested from culture vessels, counted using a Vi-CELL (Beckman Coulter, #383721), washed and resuspended in FACS buffer (PBS containing 2% FBS) at 2×106 cells/mL. Subsequently, 25 μL of cell suspension was added in triplicate to a 96-well V-bottom plate (Sarstedt, #82.1583.001) and 25 μL of each bead population was added to separate Eppendorf tubes. Anti-spike-AF647 (eBioscience, #51-6490-82) was added at a final concentration of 15 μL/mL to cells. The antibody master mix for cells additionally contained LIVE/DEAD Fixable Violet Stain (Invitrogen, #L34955) at a final concentration of 1:1000. Each bead population was correspondingly stained with anti-spike-AF647. Cells and beads were incubated with the antibodies for 1 hr at 4° C. in the dark and then washed, resuspended, and analyzed by flow cytometry on a BD FACSCelesta cytometer (BD Biosciences).

For analysis, a standard curve was generated using a spreadsheet provided by a Bangs Laboratories for the specific lot of beads and the surface antigen binding capacity (ABC) was calculated by entering the geometric means of the cell populations into this spreadsheet. ABC values represent the number of molecules of receptor expressed on the cell surface assuming a monovalent binding model.

### Binding Assessment of Variants to Wild Type and Mutant Spike by Flow Cytometry

Spike+HEK2936e and negative control mock GFP HEK2936e cells were pre-mixed in equivalent volume (50,000 cells of each population), incubated in a two-step staining protocol and analyzed by flow cytometry to evaluate the binding of (i) myc-tagged ACE2 decoy constructs, (ii) untagged IgG benchmarks, or (iii) recombinant ACE2 (Acro Biosystems, #AC2-H82E6) to ectopically expressed full length wild type or mutant SARS-CoV-2 spike protein. Test myc-tagged ACE2 decoy constructs or recombinant ACE2 were diluted three-fold in FACS buffer along 7 points from a top concentration of 50 nM in a 96 well plate (Sarstedt, #82.1583.001). Spike and GFP+ cells were thawed and resuspended to 5 million cells/mL in FACS buffer, and subsequently 20 μL of 1:1 pre-mixed suspension was aliquoted per well into assay plates containing test variants. To verify spike expression by the transfected cells, a control well containing only spike+ cells without test variant was added to the plate.

Samples were incubated for 45 minutes at 4° C. During the primary incubation, the unconjugated anti-myc secondary (clone 4A6, mouse monoclonal IgG1, Millipore, #05-724-25UG) was labelled with AF647 using a Zenon™ Mouse IgG1 Labeling Kit AF647 (Thermo, #Z25008) according to the manufacturer's protocol.

To fluorochrome label the unconjugated anti-MYC, the antibody was first diluted to 1 mg/mL. Component A (Labelling Reagent) was added, and the sample was incubated for 5 minutes at room temperature. Component B (Blocking Reagent) was then added to the mixture containing Component A and anti-myc, and the sample was incubated for an additional 5 minutes at room temperature. After 45 minutes of incubation, test construct was washed from the cells with 200 μL/well of FACS buffer and cells were resuspended in 20 μL of 1 μL/mL (final concentration) of the appropriate secondary detection antibody plus LIVE/DEAD Fixable Viability dye to 1:1000 final concentration. Secondary antibodies include Zenon labelled anti-MYC-AF647, streptavidin AF647 (Biolegend, #405237), or goat anti-human IgG Fc AF647 (Jackson ImmunoResearch, #109-606-170). Anti-spike AF647 (eBioscience, #51-6490-82) was added to the control well containing spike+ cells alone to measure spike expression. Samples were incubated with secondary for 45 minutes at room temperature.

Following incubation, plates were washed twice in 200 μL/well FACS buffer and analyzed on a Celesta flow cytometer. The resulting data was reported as raw geometric mean value or as fold over mock GFP (background); calculated in Formula A below. A normalization technique was applied to account for the difference in secondary antibody applied as calculated using Formula B.

Fold over background=AF647 geometric mean spike+cellsAF647 geometric mean mock GFP+cells/AF647 geometric mean spike+cellsAF647 geometric mean mock GFP+cells Formula A:

Normalization ratio=Geometric mean MYC-tagged ACE2 IgG1 FSA (anti-human IgG Fc AF647 detection)/Geometric mean MYC-tagged ACE2 IgG1 FSA (anti-myc AF647 detection) Geometric mean MYC-tagged ACE2 IgG1 FSA (anti-human IgG Fc AF647 detection)/Geometric mean MYC-tagged ACE2 IgG1 FSA (anti-myc AF647 detection)

Normalized data=Geometric mean MYC-tagged ACE2 IgG1 FSA (anti-human IgG Fc AF647 detection)/Average normalization ratio value Geometric mean MYC-tagged ACE2 IgG1 FSA (anti-human IgG Fc AF647 detection)/Average normalization ratio value Formula B:

The normalization ratio across each tested concentration was averaged and used to calculate the normalized data value for each titration point.

### Results

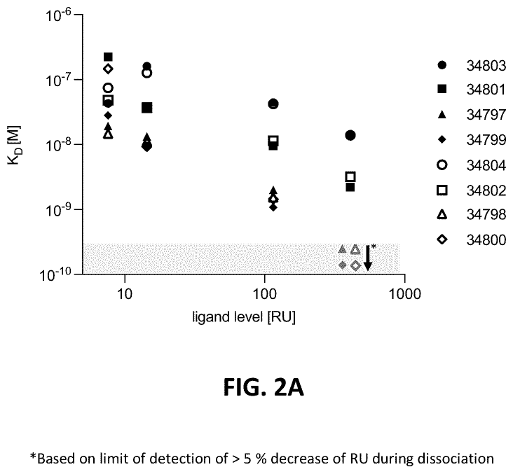

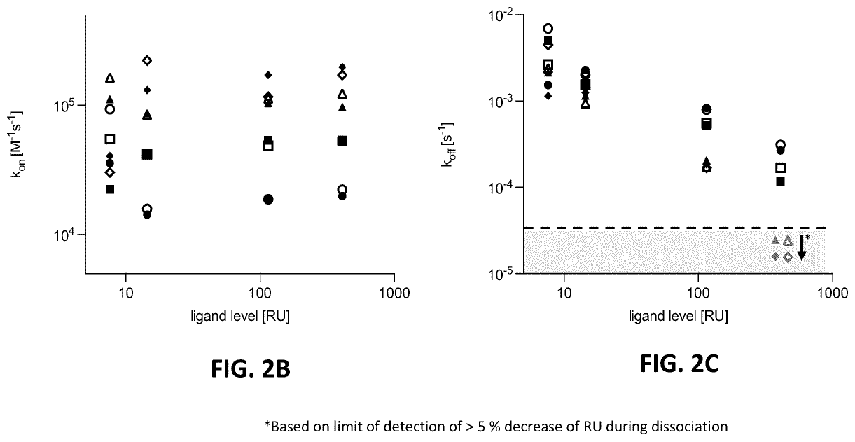

All ACE2 decoy constructs (ACE2 valency given as a number in parentheses: v34803 (1), 34801 (2), v34797 (4) and v34799 (10), 34804 (KO, 1), 34802 (KO, 2), v34798 (KO, 4) and v34800 (KO, 10)) that were assessed for binding to WT SARS-CoV-2 Spike by SPR showed association while the valency of the construct correlated with the measured affinity, whereby higher valency constructs showed higher apparent affinity (see, FIGS. 2A-2C). As further shown in TABLE 3, all multivalent ACE2 decoys demonstrated additional apparent affinity increases at higher immobilization levels of spike RBD due to increased off-rates, indicating the contribution of avidity. In contrast, the monovalent construct v34803 showed no further affinity enhancements with increased ligand density due to the independence of 1:1 binding kinetics to ligand concentrations.

Multivalent constructs based on an IgM scaffold showed the highest apparent affinity, followed by IgA and IgG, while monovalent, IgG based variants showed the lowest apparent affinity. Notably, the increased apparent affinity of higher valency constructs was more apparent at higher immobilization levels (i.e., higher ligand level) of Spike RBD, demonstrating that the increase in binding affinity of constructs with higher valency for ACE2 (e.g., deca- and dodecavalent constructs) compared to constructs with lower valency was due to avidity. At the highest Spike RBD densities tested, the dissociation rate of IgA and IgM based constructs dropped below the detection limit of the experiment. When sequences of ACE2 catalytic mutants (e.g., 34800) were used in the constructs, affinities to Spike were similar to decoys based on WT ACE2 (e.g., 34799 or 36708).

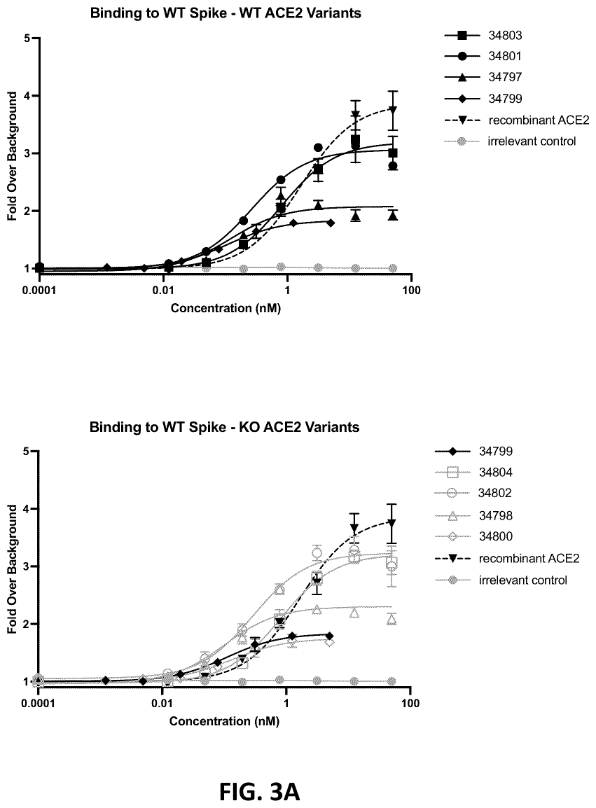

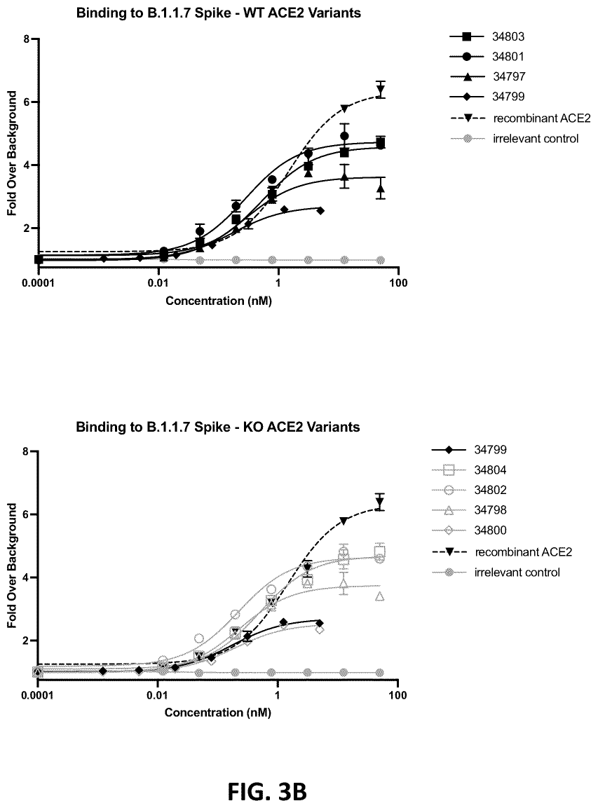

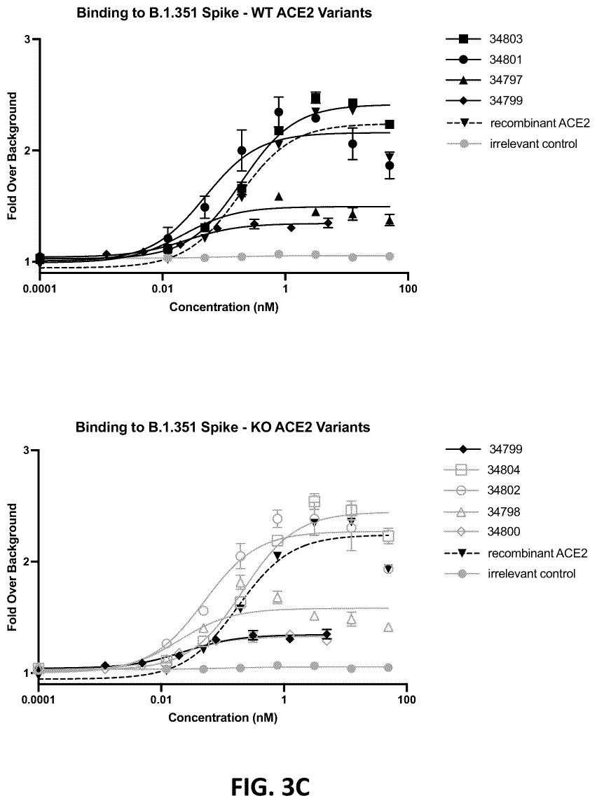

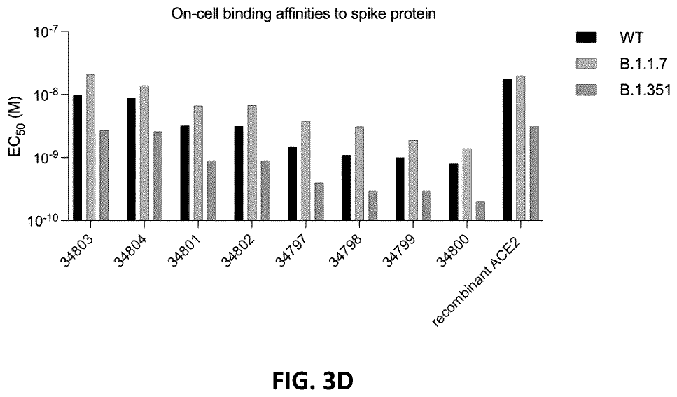

Trends seen in SPR binding experiments were confirmed when binding of these polypeptide constructs was measured to cells transfected with WT SARS-CoV-2 Spike as well as Spike from SARS-CoV-2 variants of concern (e.g., B.1.1.7, B.1.351) by flow cytometry (see, FIGS. 3A-3D). Notably, as seen in SPR, constructs using ACE2 catalytic mutants performed similarly to those using WT ACE2, and higher valency correlated with stronger binding as evidenced by a lower EC₅₀. Bₘₐₓ was also reduced for the higher valency constructs based on IgA and IgM scaffolds compared to those with lower valency based on IgG Fc domains, further corroborating the avidity seen in binding potency.

The decavalent and dodecavalent IgM-based ACE2 decoy constructs (e.g., v34799, 36708, 36709 and v34800) were confirmed as the highest-affinity constructs, followed by IgA and IgG, while monovalent IgG showed the lowest affinity. Additionally, when the Spike sequence variants with a reported higher affinity for ACE2 were used (e.g., B.1.351, B.1.1.7) a higher apparent affinity was seen in on-cell binding by the ACE2 decoys when compared to the binding experiment using WT Spike.

Specifically, the decavalent IgM ACE2 construct had the highest apparent affinity of <0.097 nM, followed by 0.564 nM for the tetravalent IgA ACE2 construct, 3.38 nM for the bivalent IgG ACE2 construct, and lastly the monovalent (OAA) ACE2 decoy construct bound the weakest with a K_{D} of 81.2 nM.

This example illustrates that the ACE2 decoy polypeptide constructs of the present disclosure, also referred throughout herein as ACE2 decoy constructs or simply polypeptide constructs, do not lose their ability to bind mutant Spike proteins of other SARS-CoV-2 strains, as long as those other SARS-CoV-2, SARS-CoV, coronavirus or any other virus utilizes ACE2 binding to obtain entry into the host cell of a subject.

TABLES 3 and 4 below show the numerical values measured in the spike protein binding experiments described herein.

TABLE 3
Assessment of Binding of Polypeptide Constructs to Spike Protein using SPR

| | K_{D} | | | | Kₐ | |
|---|---|---|---|---|---|---|
| | Spike RBD immobilization level (RU) | | | | | |
| TA^{#} | 7.6 | 14.4 | 115 | 409 | 7.6 | 14.4 |
| 34803 | 4.3E−08 | 1.6E−07 | 4.4E−08 | 1.4E−08 | 3.6E+04 | 1.4E+04 |
| 34804 | 7.5E−08 | 1.3E−07 | 4.2E−08 | 1.4E−08 | 9.3E+04 | 1.6E+04 |
| 34801 | 2.2E−07 | 3.8E−08 | 9.6E−09 | 2.2E−09 | 2.3E+04 | 4.2E+04 |
| 34802 | 4.8E−08 | 3.7E−08 | 1.1E−08 | 3.2E−09 | 5.5E+04 | 4.2E+04 |
| 34797 | 1.9E−08 | 1.3E−08 | 2.0E−09 | <5.1E−10* | 1.1E+05 | 8.7E+04 |
| 34798 | 1.5E−08 | 1.1E−08 | 1.5E−09 | <4.2E−10* | 1.6E+05 | 8.4E+04 |
| 34799 | 2.8E−08 | 9.6E−09 | 1.1E−09 | <2.5E−10* | 4.1E+04 | 1.3E+05 |
| 34800 | 1.5E−07 | 9.3E−09 | 1.5E−09 | <2.9E−10* | 3.0E+04 | 2.2E+05 |

| | Kₐ | | K_{d} | | | |
|---|---|---|---|---|---|---|
| | Spike RBD immobilization level (RU) | | | | | |
| TA^{#} | 115 | 409 | 7.6 | 14.4 | 115 | 409 |
| 34803 | 1.9E+04 | 2.0E+04 | 1.5E−03 | 2.3E−03 | 8.3E−04 | 2.7E−04 |
| 34804 | 1.9E+04 | 2.2E+04 | 7.0E−03 | 2.0E−03 | 7.9E−04 | 3.1E−04 |
| 34801 | 5.4E+04 | 5.4E+04 | 5.0E−03 | 1.6E−03 | 5.2E−04 | 1.2E−04 |
| 34802 | 4.9E+04 | 5.3E+04 | 2.7E−03 | 1.6E−03 | 5.5E−04 | 1.7E−04 |
| 34797 | 1.0E+05 | 9.8E+04 | 2.2E−03 | 1.2E−03 | 2.1E−04 | <5.0E−05* |
| 34798 | 1.1E+05 | 1.2E+05 | 2.4E−03 | 9.5E−04 | 1.7E−04 | <5.0E−05* |
| 34799 | 1.7E+05 | 2.0E+05 | 1.1E−03 | 1.3E−03 | 1.9E−04 | <5.0E−05* |
| 34800 | 1.2E+05 | 1.7E+05 | 4.5E−03 | 2.1E−03 | 1.7E−04 | <5.0E−05* |

^{#}“TA” refers to “test article” and the ACE2 decoy constructs tested
*Based on limit of detection of >5% decrease of RU during dissociation

TABLE 4
On-cell Binding of ACE2 Decoy Polypeptide Constructs to
SARS-CoV-2 Spike Protein Assessed by Flow Cytometry

| | Apparent K_{D} (M) | | | Bₘₐₓ (Fold over Background) | | |
|---|---|---|---|---|---|---|
| | SARS-CoV-2 Spike Variant | | | SARS-CoV-2 Spike Variant | | |
| Construct # | WT | B.1.1.7 | B.1.351 | WT | B.1.1.7 | B.1.351 |
| 34803 | 9.80E−09 | 2.10E−08 | 2.70E−09 | 3.3 | 6.1 | 2.8 |
| 34804 | 8.80E−09 | 1.40E−08 | 2.60E−09 | 3.4 | 5.9 | 2.9 |
| 34801 | 3.30E−09 | 6.70E−09 | 9.00E−10 | 3.2 | 5.6 | 2.5 |
| 34802 | 3.20E−09 | 6.80E−09 | 9.00E−10 | 3.3 | 5.8 | 2.6 |
| 34797 | 1.50E−09 | 3.80E−09 | 4.00E−10 | 2.7 | 4.7 | 1.9 |
| 34798 | 1.10E−09 | 3.10E−09 | 3.00E−10 | 2.7 | 4.7 | 2 |
| 34799 | 1.00E−09 | 1.90E−09 | 3.00E−10 | 2.4 | 3.8 | 1.7 |
| 34800 | 8.00E−10 | 1.40E−09 | 2.00E−10 | 2.2 | 3.3 | 1.6 |
| Acro Biosystems | 1.80E−08 | 2.00E−08 | 3.20E−09 | 3.3 | 6.1 | 2.5 |
| (#AC2-H82E6) | | | | | | |

Additionally, binding of the mono- and multivalent polypeptide constructs was also measured for SARS-CoV-2 Delta and Omicron variant RBDs and, similarly, apparent affinity correlated with ACE2 valency (see, e.g., TABLE 5). Across the different SARS-CoV-2 variant RBDs tested, decavalent binding of the IgM-based ACE2 decoy polypeptide construct (v36708) increased the apparent affinity to spike RBD by two orders of magnitude compared with monovalent OAA ACE2 construct, and also appeared to be agnostic of SARS-CoV-2 variant RBD tested (e.g., no significant loss in affinity when tested in WT, Delta (B.1.617.2) and Omicron (B.1.1.529) variant).

The binding affinities of the two anti-spike mAbs used in Regeneron's REGEN-COV cocktail granted EUA, casirivimab (REGN10933) and imdevimab (REGN1987), for SARS-CoV-2 variant RBDs were also measured by SPR for comparison. Both benchmark antibodies bound to SARS-CoV-2 Wuhan (WT) and Delta spike RBDs with sub-nanomolar affinities, similar to the decavalent IgM ACE2 construct (TABLE 5). In contrast, REGEN-COV mAb Imdevimab suffered complete loss in binding to SARS-CoV-2 Omicron spike RBD and Casirivimab K_{D} was 1.18 μM, while IgM ACE2 decoy retained high binding affinity with K_{D} of 2.4 nM (TABLE 5).

TABLE 5
Binding Assessment to Spike proteins from Relevant Variants

| | SARS-CoV-2 strain | | |
|---|---|---|---|
| | Wuhan (WT) | Delta Variant | Omicron Variant |
| Construct | K_{D} (M)^{a} | K_{D} (M) | K_{D} (M) |
| v36708 | 9.66E−11 | 4.85E−10 | 2.44E−09 |
| v36706 | 3.54E−10 | 7.99E−10 | 2.74E−09 |
| v36710 | 3.38E−09 | 6.02E−09 | 2.29E−08 |
| v36712 | 8.12E−09 | 1.13E−08 | 1.29E−07 |
| REGN10933 | 7.08E−11 | 6.96E−11 | 1.18E−06^{b} |
| REGN10987 | 8.69E−11 | 1.05E−10 | No binding |
| IgM ctrl | No binding | No binding | No binding |
| IgG ctrl | No binding | No binding | No binding |

^{a}Values derived using a 1:1 model and reported as apparent K_{D} values for multivalent constructs
^{b}steady state fitting

Generally, on-cell binding of the mono- and multivalent ACE2 decoy polypeptide constructs described herein to SARS-CoV-2 Wuhan (WT) spike protein transfected mammalian cells had consistent rank order as shown by the SPR spike RBD affinity measurements. Lower Bₘₐₓ was observed for the higher valency ACE2 decoys indicating that fewer molecules are required to saturate all on-cell binding sites consistent with a mechanism of multivalent binding to multiple surface spike protein RBDs by a single multivalent construct.

### Example 3

### Neutralization of Spike-ACE2 Protein Interactions Using Multivalent ACE2 Decoy Polypeptide Constructs

The ability of the ACE2 decoy polypeptide constructs described and produced herein, e.g., those described in EXAMPLE 1, to disrupt the Spike protein-ACE2 protein interaction between SARS-CoV-2 viral particles (comprising spike proteins) and target cells (expressing ACE2) was assessed in vitro using an electrochemiluminescence-based multiplex immune assay (Mesoscale Discovery, MSD).

### Spike-ACE2 MSD Surrogate Neutralization Assay

The ability of the test constructs (v34803 (1), 34801 (2), v34797 (4) and v34799 (10), 34804 (KO, 1), 34802 (KO, 2), v34798 (KO, 4) and v34800 (KO, 10); ACE2 valency given as a number in parentheses) to inhibit the binding of SULFO-tag labelled recombinant ACE2 to plates pre-coated with WT or mutant (e.g., B.1.1.7, B.1.351, and P.1) spike protein, S1 RBD, or nucleocapsid was evaluated using a 10 spot V-Plex® ACE2 Neutralization Kit SARS-CoV2 Panel 7 (Mesoscale Discovery, #K15440U). The assay was performed according to the manufacturer's protocol using a 96 well format.

Prior to addition of test variants, 150 μL/well of Blocking Solution A was added to the V-Plex plate. The plate was sealed and incubated at room temperature for 30 mins on a shaking platform set to 700 rpm. During this time, construct and calibrator stock dilutions were prepared in Diluent 100 (MSD, #R50AA-3) in a non-binding 96 well plate (Sarstedt, #82.1583.001). The Calibrator stock was diluted 4-fold along 7 points, and the constructs were titrated 3-fold, 7 points, from a top concentration of 133 nM and 50 nM, respectively. After incubation with blocking solution, the plate was washed 3 times with a BioTek EL-405 (300 uL/well) in wash buffer (PBS+0.05% Tween-20). Immediately after washing, 25 μL of diluted constructs or calibrator were added to the V-Plex plate.

For assay controls, 25 μL of Diluent 100 alone (negative control) or 25 μL of 50 nM rACE2 (recombinant ACE2, positive control) were added in duplicate. The plate was sealed and incubated at room temperature on a shaking platform set to 700 rpm for 1 hr. A 1× working stock of detection solution was prepared with SULFO-tag labelled recombinant ACE2 (200× stock) in Diluent 100 and 25 μL/well was added to the plate containing variant and calibrator. The plate was sealed and incubated at room temperature on a shaking platform at 700 rpm for 1 hr and then washed 3× using a BioTek EL-405 (300 μL/well) in wash buffer (PBS+0.05% Tween-20). Following this, 150 μL/well of MSD Gold Read Buffer B (MSD, #R60AM-4) was added and the plate was read immediately on the Meso Sector S 600. The resulting data was reported as raw signal intensity.

### Results

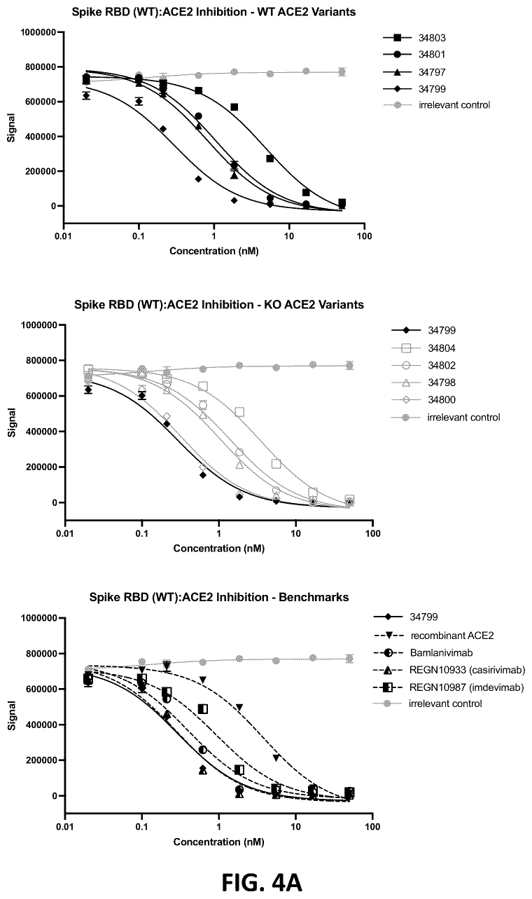

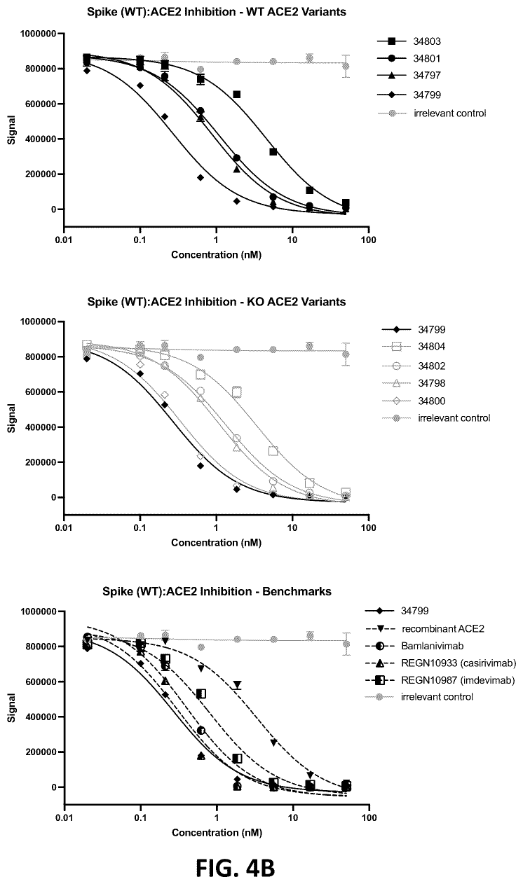

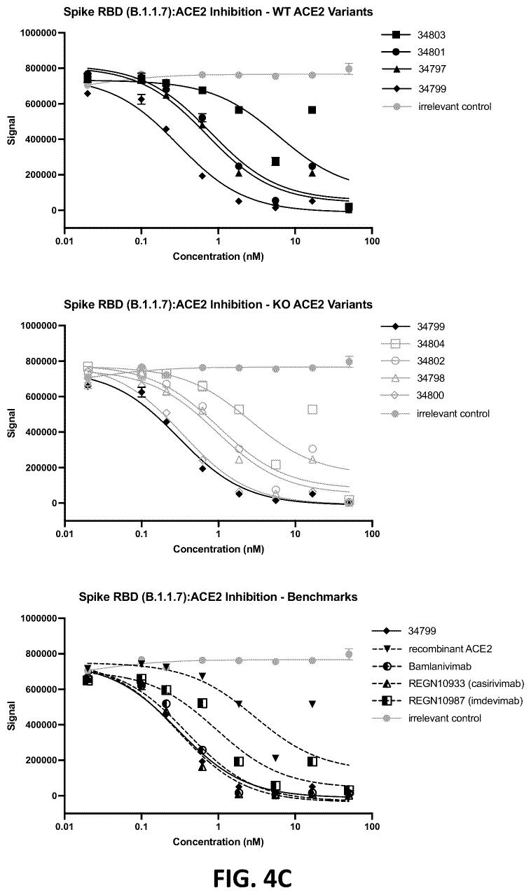

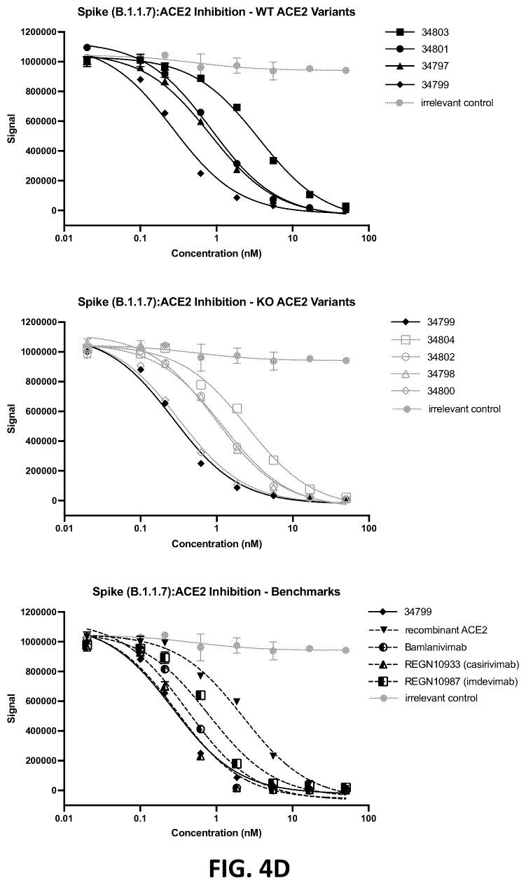

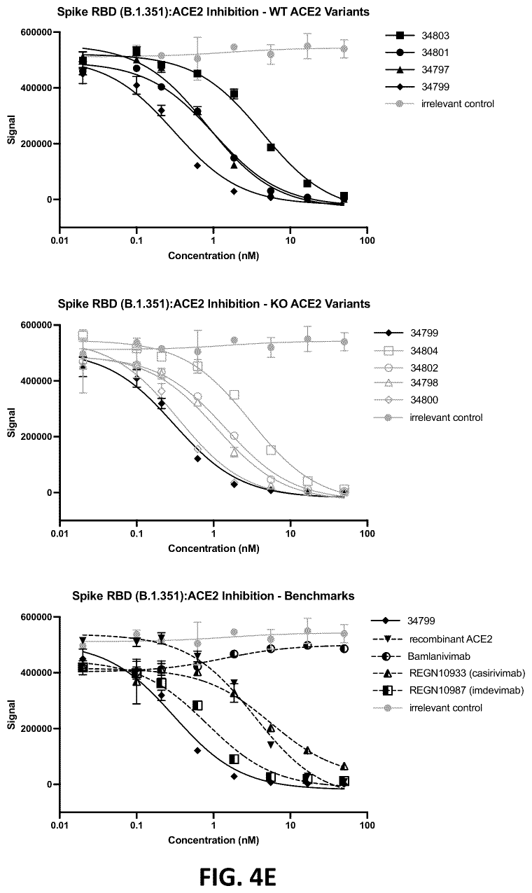

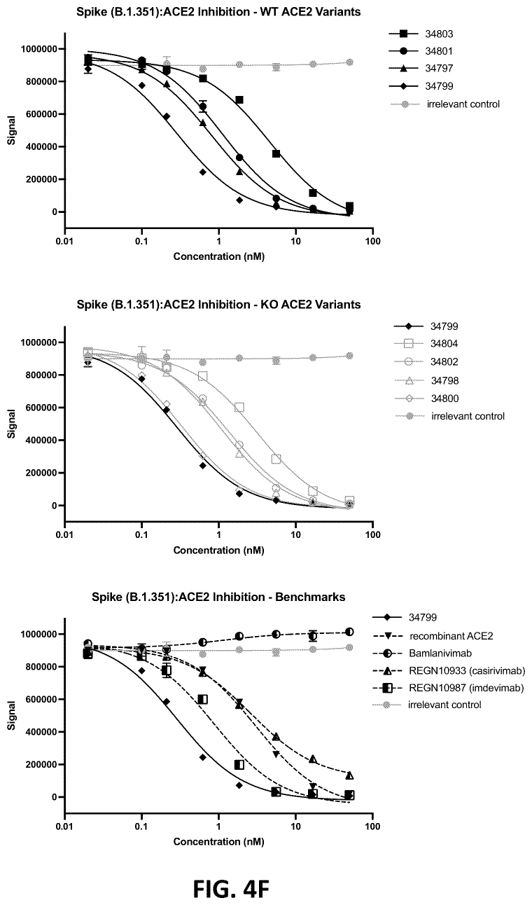

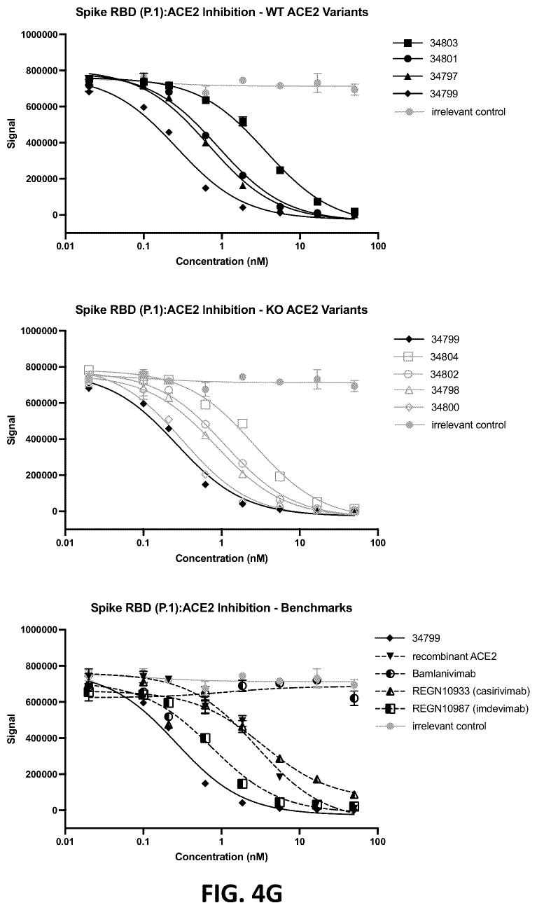

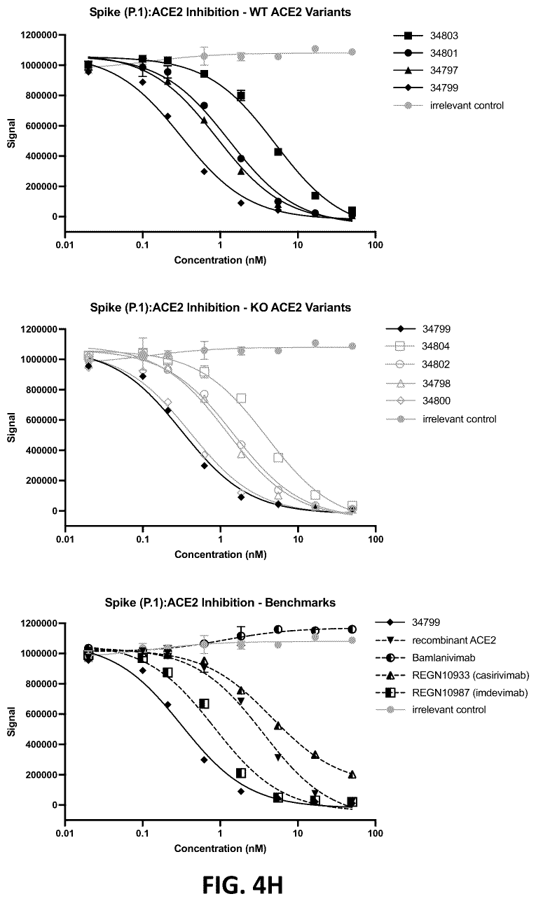

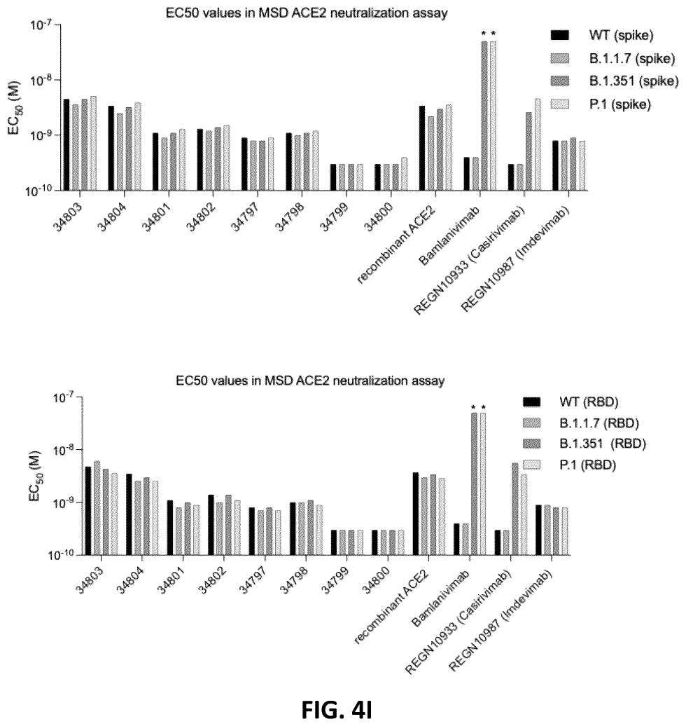

All ACE2 decoy polypeptide constructs of this disclosure (v34803 (1), 34801 (2), v34797 (4) and v34799 (10), 34804 (KO, 1), 34802 (KO, 2), v34798 (KO, 4) and v34800 (KO, 10)) that were assessed in this MSD based neutralization assay showed activity for neutralizing the ACE2-to-SARS-CoV-2 Spike protein interaction (see, e.g., FIGS. 4A-4I, TABLE 6). The decavalent polypeptide constructs based on IgM scaffolds (e.g., v34799, 36708, 36709 and v34800) showed the highest potency (e.g., with half-maximal inhibitory concentration (IC₅₀) ranging between 0.23-0.47 nM across all variant spike proteins tested for constructs v34799 and v34800) for neutralizing ACE2-to-SARS-CoV-2 Spike protein interaction, respectively, see, e.g., FIG. 4I), followed by IgA and IgG, while monovalent IgG based variants (e.g., v34803 and v34804, respectively) showed the lowest potency. These data correlated well with and further supported the results that were seen in corresponding binding assays, e.g., those described in EXAMPLE 2. This potency rank order was consistent with SPR and on-cell binding measurements supporting the finding that higher ACE2 valency affords avidity that can drive high affinity binding and increase neutralization efficacy. Additionally, all ACE2 decoys retained similar neutralization potency agnostic of SARS-CoV-2 variant spike protein tested. Similarly, ACE2 catalytic mutants (e.g., decavalent construct v34800 (KO) vs. 34799) showed comparable potency to their WT ACE2 equivalents.

Importantly, while anti-Spike benchmark antibodies showed high potency at inhibiting the ACE2 to SARS-CoV-2 Spike interaction for WT and B.1.1.7 SARS-CoV Spike (0.3 nM-0.9 nM), Bamlanivimab and REGN10933 (Casirivimab), for example, showed no or significantly reduced binding to variant spike protein of, e.g., the omicron variant and/or subvariants (e.g., omega subvariants BA.3, BA.4, BA.5, etc.), see., e.g., data summary TABLE 9. The same was observed, for most virus variants, for the antibodies etesevimab and sotrovimab, see., e.g., data summary TABLE 9. These trends were also observed, in most cases, for other SARS-CoV-2 Spike variants of concern (B.1.1.7, B.1.351 and P.1). Moreover, and in contrast to the multivalent ACE2 decoy constructs described herein, numerous clinically relevant anti-spike benchmark antibodies exhibited virus variant-dependent neutralization capabilities. As an example, IC₅₀ values for REGEN-COV against Wuhan (WT), Alpha, Beta, Gamma, and Delta SARS-COV-2 spike protein ranged from about 0.05 nM to 0.40 nM but decreased against Omicron to 13.1 nM (TABLE 7).

Taken together, all ACE2 decoy constructs tested in this study showed potency for neutralizing the ACE2 to SARS-CoV-2 Spike interaction across all tested Spike variants (WT, B.1.1.7, B.1.351, P.1). The ACE2 decoy molecules tested in this study did not lose their ability to neutralize infections by other mutant SARS-CoV-2 strains, as long as the mutant SARS-CoV-2 strains (also referred to herein as variants) rely on ACE2 binding for viral entry into the host cell of a subject. Furthermore, the decavalent ACE2 decoy polypeptide constructs based on IgM scaffolds (e.g., v34799, 36708, 36709 and v34800) showed the highest potency for neutralizing ACE2-to-SARS-CoV-2 Spike interaction.

TABLES 6 and 7 below shows the numerical values measured in the MSD neutralization assay experiments described herein.

TABLE 6
SARS-CoV-2 Spike-ACE2 MSD Neutralization Assay Data for Polypeptide Constructs tested in this Study.

| | IC₅₀ (M) | | | | | | | |
|---|---|---|---|---|---|---|---|---|
| | SARS-CoV-2 Spike Variant | | | | | | | |
| | | WT | | B.1.1.7 | | B.1.351 | | P.1 |
| Variant | WT | (RBD only) | B.1.1.7 | (RBD only) | B.1.351 | (RBD only) | P.1 | (RBD only) |
| 34803 | 4.50E−09 | 4.80E−09 | 3.60E−09 | 6.10E−09 | 4.50E−09 | 4.30E−09 | 5.10E−09 | 3.60E−09 |
| 34804 | 3.40E−09 | 3.50E−09 | 2.50E−09 | 2.60E−09 | 3.20E−09 | 3.00E−09 | 3.90E−09 | 2.60E−09 |
| 34801 | 1.10E−09 | 1.10E−09 | 9.00E−10 | 8.00E−10 | 1.10E−09 | 1.00E−09 | 1.30E−09 | 9.00E−10 |
| 34802 | 1.30E−09 | 1.40E−09 | 1.20E−09 | 1.00E−09 | 1.40E−09 | 1.40E−09 | 1.50E−09 | 1.10E−09 |
| 34797 | 9.00E−10 | 8.00E−10 | 8.00E−10 | 7.00E−10 | 8.00E−10 | 8.00E−10 | 9.00E−10 | 7.00E−10 |
| 34798 | 1.10E−09 | 1.00E−09 | 1.00E−09 | 1.00E−09 | 1.10E−09 | 1.10E−09 | 1.20E−09 | 9.00E−10 |
| 34799 | 3.00E−10 | 3.00E−10 | 3.00E−10 | 3.00E−10 | 3.00E−10 | 3.00E−10 | 3.00E−10 | 3.00E−10 |
| 34800 | 3.00E−10 | 3.00E−10 | 3.00E−10 | 3.00E−10 | 3.00E−10 | 3.00E−10 | 4.00E−10 | 3.00E−10 |
| recombinant | 3.40E−09 | 3.70E−09 | 2.20E−09 | 3.00E−09 | 3.00E−09 | 3.40E−09 | 3.60E−09 | 2.90E−09 |
| ACE2 | | | | | | | | |
| Bamlanivimab | 4.00E−10 | 4.00E−10 | 4.00E−10 | 4.00E−10 | — | — | — | — |
| REGN10933 | 3.00E−10 | 3.00E−10 | 3.00E−10 | 3.00E−10 | 2.60E−09 | 5.60E−09 | 4.60E−09 | 3.40E−09 |
| (Casirivimab) | | | | | | | | |
| REGN10987 | 8.00E−10 | 9.00E−10 | 8.00E−10 | 9.00E−10 | 9.00E−10 | 8.00E−10 | 8.00E−10 | 8.00E−10 |
| (Imdevimab) | | | | | | | | |
| Irrelevant | — | — | — | — | — | — | — | — |
| Control | | | | | | | | |

TABLE 7
Summary of IC₅₀ (MSD and Pseudovirus Neutralization)
and MN₅₀ (live virus neutralization) for Selected Constructs
and Clinical Benchmark Antibodies REGEN-COV.
IC₅₀ or MN₅₀ (nM)

| SARS-CoV-2 | | | | | REGEN- |
|---|---|---|---|---|---|
| variant | Experiment | v36712 | v36710 | v36708 | COV |
| Wuhan (WT) | MSD | 1.19 | 3.39 | 0.24 | 0.07 |
| | Pseudovirus | Not | 7.67 | 0.02 | 0.09 |
| | | tested | | | |
| Delta | MSD | 1.41 | 3.62 | 0.25 | 0.05 |
| | Pseudovirus | Not | 1.56 | 0.03 | 0.05 |
| | | tested | | | |
| | Live Virus | Not | 1.80 | 0.08 | 0.05 |
| | | tested | | | |
| Omicron | MSD | 2.67 | 8.58 | 0.47 | 13.13 |
| | Pseudovirus | Not | 8.08 | 0.03 | No activity |
| | | tested | | | |

### Example 4

### SARS-CoV-2 Spike Pseudovirus Neutralization Using ACE2 Decoy Polypeptide Constructs

The ability of multivalent ACE2 decoy polypeptide constructs described and produced herein, e.g., those described in EXAMPLE 1, to neutralize an infection of ACE2-bearing target cells by a SARS-CoV-2 pseudotyped lentivirus was assessed.

### Pseudovirus Neutralization Assay

A SARS-CoV-2 Pseudovirus Neutralization Assay (GenScript kit, #SC2087A) was used to quantify the inhibition of pseudoviral entry into ACE2 expressing target cells in the presence of the multivalent ACE2-containing decoy polypeptide constructs tested herein. In this assay, the pseudovirus expressed full length wild type spike glycoprotein and delivered a luciferase reporter upon entry into target cells. This protocol was adapted from the manufacture's protocol and transitioned from 96 to 384 well format (Thermo, #165610).

The test constructs and the positive control provided in the kit (bivalent ACE2 Fc, Novoprotein, #C05Y) were diluted to a top concentration of 300 nM (or 30 nM for IgM) in OptiMEM, 4-fold along 8 points and 12.5 μL/well was added to the assay plate. Pseudovirus was then thawed in a water bath at 37° C. with gentle agitation, and aliquoted into 3 mL of OptiMEM. Following this, 12.5 μL of pseudovirus was added to the assay plate containing diluted test constructs, and the samples were incubated for 1 hour (hr) at room temperature. During the incubation time, Opti-HEK293/ACE2 cells supplied with the GenScript kit were thawed in a water bath at 37° C. with gentle agitation and aliquoted into 4 mL of pre-warmed complete growth media (DMEM (Thermo, #11995081)+10% FBS). Viable cells were counted using a Vi-CELL (Beckman Coulter, #383721), washed, and resuspended to a density of 300,000 cells/mL in complete growth media and incubated at 37° C., 5% CO₂ until use. After 1 hr incubation of pseudovirus with tested ACE2 decoy polypeptide constructs, 25 μL/well of Opti-HEK293/ACE2 cells were added to the plate, and samples were incubated for 24 hr at 37° C. with 5% CO₂. Following this incubation, 25 μL/well of pre-warmed complete growth media was added and samples were again incubated for 24 hrs at 37° C., 5% CO₂. After the second 24 hr incubation, 75 μL of media was removed from all assay wells, and 25 μL of Bio-Glo™ Luciferase Reagent (Promega, #G7940) was added to all wells. The plate was incubated at room temperature for 5 minutes and then read in luminescence mode on a Synergy H1 plate reader (Biotek). Data is reported as raw luminescence signal intensity (RLU), or percentage inhibition calculated using Formula C.

$$Inhibition rate ( \% ) = 1 - [ ( RLU positive control - blank control ) / ( RLU negative control - blank control ) ] \times 100 \qquad Formula C$$

### Results

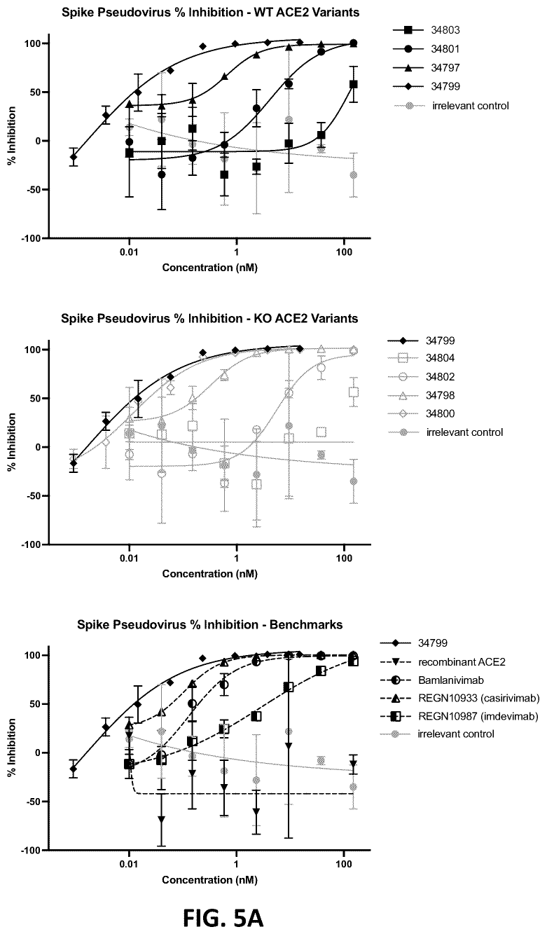

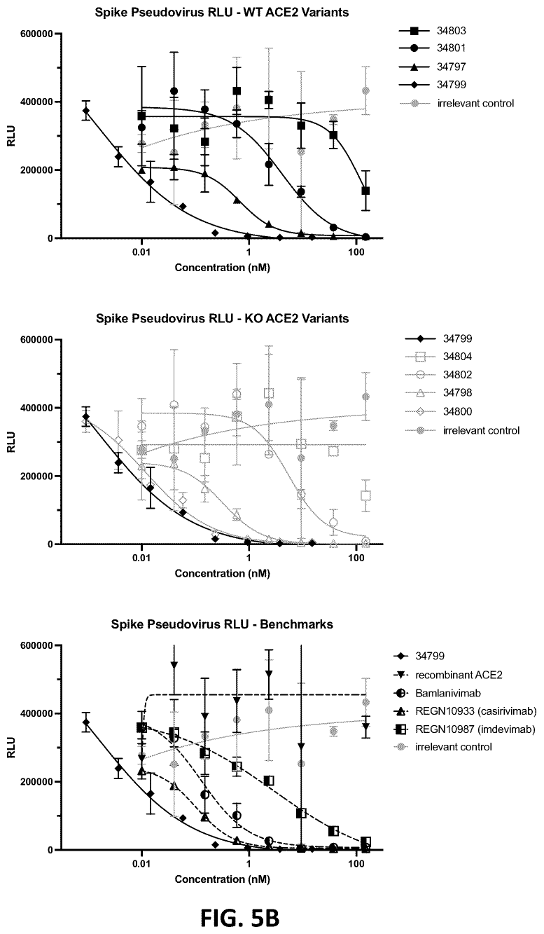

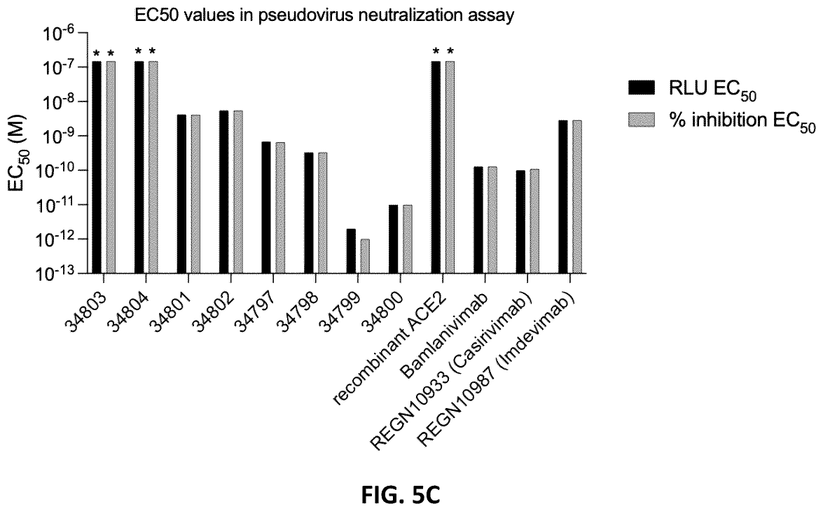

All ACE2 decoy constructs assessed in the MSD based neutralization assay showed activity for neutralizing entry of Spike pseudotyped lentiviruses into an ACE2 positive cell line (see, e.g., FIGS. 5A-5C, TABLE 8). The decavalent ACE2 decoy constructs based on IgM scaffolds showed the highest potency (e.g., with EC₅₀ values of ≤10⁻¹¹ for decavalent constructs v34799 and v34800, respectively), followed by IgA and IgG based constructs, while monovalent, IgG based constructs showed the lowest potency (e.g., v34803 and v34804). This rank order correlated with what was found in binding and MSD-based binding inhibition assays described in EXAMPLE 2 and EXAMPLE 3, respectively.

As previously shown, the ACE2 catalytic mutants (e.g., v34800) showed also in this study a comparable potency to their WT ACE2 equivalents (e.g., v34799 and 36708). Furthermore, IgM based ACE2 decoy constructs (34799, 36708, 36709 and 34800) showed higher, variant-agnostic potency in the pseudovirus neutralization assay than all clinical benchmarks tested (e.g., Bamlanivimab, REGN10987 (Imdevimab), REGN10933 (Casirivimab)). When tested against virus particles pseudotyped with Spike mutants found in Variants of Concern (e.g., variants B.1.1.7, B.1.351, P.1, B.1.617.2), all ACE2 decoy constructs showed no or at least no significant (<5%) loss in potency while some clinical benchmark anti-spike antibodies showed significantly lowered potency, as seen previously in the MSD-based neutralization assay in EXAMPLE 3 (e.g., Bamlanivimab against B.1.351 and P.1, etc.). Hence, the ACE2 decoy constructs described in this example did not lose their ability to neutralize infections by other mutant SARS-CoV-2 strains (also referenced throughout this application as virus variants), as long as these SARS-CoV, SARS-CoV-2, coronavirus and any virus rely on ACE2 protein binding for viral entry into the host cell including a host cell of a human subject. For instance, the decavalent IgM-based ACE2 decoy constructs markedly shifted the neutralization potency relative to molar-matched bivalent IgG ACE2 decoy construct by an average of 185-fold for lentivirus pseudotyped with Wuhan (WT), Delta, Omicron, Alpha, Beta, or Gamma spike protein (see, e.g., TABLE 9). Notably, decavalent IgM-based ACE2 decoy constructs and REGEN-COV had neutralization potencies against Wuhan (WT) and Delta pseudovirus within the same order of magnitude, however, and in contrast to the decavalent decoy construct, REGEN-COV failed to prevent Omicron pseudotyped lentivirus from infecting ACE2-expressing cells (see, also, TABLES 8 and 9).

TABLE 8 below shows the numerical values measured in the pseudovirus neutralization assay experiments described herein.

TABLE 8
SARS-CoV-2 Spike Pseudovirus Neutralization Assay

| | RLU | %-inhibition | Bₘₐₓ |
|---|---|---|---|
| Variant | EC₅₀ (M) | EC₅₀ (M) | (% inhibition) |
| 34803 | >1.50E−07 | >1.50E−07 | — |
| 34804 | >1.50E−07 | >1.50E−07 | — |
| 34801 | 4.20E−09 | 4.10E−09 | 104 |
| 34802 | 5.50E−09 | 5.50E−09 | 95 |
| 34797 | 6.80E−10 | 6.60E−10 | 99 |
| 34798 | 3.30E−10 | 3.30E−10 | 102 |
| 34799 | 2.00E−12 | 1.00E−12 | 106 |
| 34800 | 1.00E−11 | 1.00E−11 | 102 |
| recombinant ACE2 | >1.50E−07 | >1.50E−07 | — |
| Bamlanivimab | 1.30E−10 | 1.30E−10 | 100 |
| REGN10933 (casirivimab) | 1.00E−10 | 1.10E−10 | 101 |
| REGN10987 (imdevimab) | 2.90E−09 | 2.90E−09 | 116 |
| Control | — | — | — |

### Example 5

### SARS-CoV-2 Live Virus Neutralization Using Multivalent ACE2 Decoy Polypeptide Constructs

The ability of the mono- and multivalent ACE2 decoy polypeptide constructs described and produced herein, e.g., those described in EXAMPLE 1, to neutralize an infection of ACE2 positive host cells by live SARS-CoV-2 was assessed.

### Methods

ACE2 decoy polypeptide constructs designed and produced in EXAMPLE 1 and interrogated for their binding and neutralization properties as described in EXAMPLES 2-4 were assessed for their ability to prevent entry of live SARS-CoV-2 particles into an ACE2 positive human lung epithelial cell line (Calu-3) as well as colon epithelial organoids, as previously described (see, e.g., https://www.biorxiv.org/content/-10.1101/2021.05.03.442520v1). Briefly, test articles (e.g., the tested decoy constructs) were prepared as a 2× working stock solution by titrating down an 8 point, 3 fold serial dilution followed by a 4 point 10 fold dilution series starting at 300 nM. Test samples comprising the tested constructs were further diluted 1:1 with a viral stock solution (approximately 1.0E2 TCID/100 μl) and incubated at 37° C. for an hour in a 5% CO₂ incubator. Following the virus/ACE2 decoy construct incubation, the samples were transferred to plates containing Calu-3 cell monolayers and incubated at 37° C. in a 5% CO₂ incubator for five days. To measure MN₅₀ (microneutralization concentration), plates were washed, fixed, and stained with crystal violet. The absorbance was measured at 570 nm using Spectramax ID-5 microtiter plate reader. Data was plotted using GraphPad Prism software and the MN₅₀ curves were generated using sigmoidal, 4PL non-linear equation.

A TCID₅₀ (tissue culture infectious dose) end point titre was used to titrate the virus diluted for use in the assay. Serial 10-fold dilutions of both beta and delta variants were incubated with Calu3 cells and incubated at 37° C. in a 5% CO₂ incubator for five days. The virus specific CPE (cytopathic effect) was observed microscopically for each and the TCID₅₀ was calculated using the Spearman-Karber equation to determine the titre of the virus added to the cells.

### Results

The potency rank order of the mono- and multivalent ACE2 decoy polypeptide constructs was preserved compared to binding, MSD and pseudotype neutralization assays, as established in EXAMPLES 2-4, such that the decavalent IgM-based ACE2 decoy constructs had a 22-fold enhanced SARS-CoV-2 Delta neutralizing activity compared to bivalent IgG constructs. Furthermore, decavalent IgM-based ACE2 decoy constructs showed consistent efficacy between viral strains tested and which was similar to the tested clinical benchmark REGEN-COV. REGN-COV only showed modest viral variant-dependent potency with ˜2-fold decrease in neutralization efficacy for SARS-COV-2 Beta strain compared to Delta.

TABLE 9
Summary of In Vitro Data Obtained in EXAMPLES 2-5 for Various
ACE2 Decoy Polypeptide Constructs and Clinical Benchmarks

| | Construct or | SARS-CoV-2 strain | | | | | |
|---|---|---|---|---|---|---|---|
| Assay | Antibody | WT | Alpha | Beta | Gamma | Delta | Omicron |
| SPR (K_{D}, M)^{a} | v36708 | 9.66E−11 | <1.00E−12 | <1.00E−12 | 1.99E−09 | 4.85E−10 | 2.44E−09 |
| | v36706 | 3.54E−10 | <1.00E−12 | 7.16E−10 | 2.22E−09 | 7.99E−10 | 2.74E−09 |
| | v36710 | 3.38E−09 | 2.67E−09 | 6.11E−09 | 1.57E−08 | 6.02E−09 | 2.29E−08 |
| | v36712 | 8.12E−09 | 1.11E−08 | 4.35E−08 | 4.72E−08 | 1.13E−08 | 1.29E−07 |
| | Casirivimab | 7.08E−11 | NA^{b} | NA | NA | 6.96E−11 | 1.18E−06 |
| | Imdevimab | 8.69E−11 | NA | NA | NA | 1.05E−10 | NB^{c} |
| MSD | v36708 | 0.24 | 0.23 | 0.25 | 0.29 | 0.25 | 0.47 |
| Neutralization | v36706 | 0.66 | 0.57 | 0.63 | 0.80 | 0.65 | 1.50 |
| (IC₅₀, nM) | v36710 | 3.39 | 2.81 | 3.73 | 4.00 | 3.62 | 8.58 |
| | v36712 | 1.19 | 1.05 | 1.12 | 1.18 | 1.41 | 2.67 |
| | REGEN-COV | 0.07 | 0.06 | 0.35 | 0.40 | 0.05 | 13.13 |
| | Casirivimab | 0.06 | 0.06 | 0.95 | 0.90 | 0.05 | 14.48 |
| | Imdevimab | 0.57 | 0.48 | 0.54 | 0.57 | 0.51 | 1.14 |
| | Bamlanivimab + | 0.07 | 0.08 | 0.31 | >100 | 0.16 | 0.59 |
| | Etesevimab | | | | | | |
| | Bamlanivimab | 0.10 | 0.09 | 1.59 | 0.76 | 0.32 | 1.30 |
| | Etesevimab | 0.51 | 1.06 | 1.82 | 0.08 | 0.50 | 2.69 |
| | Sotrovimab | 1.13 | 0.86 | 1.21 | 1.21 | 1.56 | 5.68 |
| Pseudovirus | v36708 | 0.02 | 0.13 | 0.18 | NA | 0.03 | 0.03 |
| Neutralization | v36706 | 0.33 | 0.06 | 0.08 | NA | NA | NA |
| (IC₅₀, nM) | v36710 | 7.67 | 7.59 | 3.33 | NA | 1.56 | 8.08 |
| | v36712 | NA | >100 | >100 | NA | NA | NA |
| | REGEN-COV | 0.09 | 0.06 | 4.90 | NA | 0.05 | NB |
| | Casirivimab | 0.05 | 0.05 | 0.00 | NA | NA | NA |
| | Imdevimab | 4.76 | 0.13 | 41.14 | NA | NA | NA |
| | Bamlanivimab + | NA | NA | NA | NA | NA | NA |
| | Etesevimab | | | | | | |
| | Bamlanivimab | 2.29 | 1.90 | >100 | NA | NA | NA |
| | Etesevimab | NA | NA | NA | NA | NA | NA |
| | Sotrovimab | 0.11 | 3.32 | 0.01 | NA | NA | NA |
| Live virus | v36708 | NA | NA | 0.05 | NA | 0.08 | NA |
| neutralization | v36706 | NA | NA | 0.24 | NA | 0.37 | NA |
| (MN₅₀, nM) | v36710 | NA | NA | NA | NA | 1.80 | NA |
| | v36712 | NA | NA | NA | NA | NA | NA |
| | REGEN-COV | NA | NA | 0.08 | NA | 0.05 | NA |

^{a}Alpha, beta, gamma SPR values were measured using myc-tagged versions of ACE2 decoys (v34799, v34797, v34801, and v34803)
^{b}NA (not available), sample were not measured
^{c}NB (no binding), sample was measured but found not to bind or neutralize in assay

### Example 6

### Neutralization of SARS-CoV-2 Infection In Vivo Using a Decavalent ACE2 Decoy Polypeptide Construct

This example describes an in vivo study that assessed the ability of various ACE2 decoy polypeptide constructs described and produced herein, including the decavalent IgM-based construct v36708, to neutralize a live SARS-CoV-2 viral infection in a hamster model of infection.

### Methods

Male LVG Golden Syrian Hamsters (81-90 g) were obtained from Charles River Laboratories (Saint-Constant, Canada). Animals were maintained at the small animal facility of the National Research Council Canada (NRC) in accordance with the guidelines of the Canadian Council on Animal Care. All procedures performed on animals in this study were approved by the Institutional Review Board (NRC Human Health Therapeutics Animal Care Committee) and covered under animal use protocol 2020.06.

SARS-CoV-2 isolate hCoV-19/USA/MD-HP05647/2021 (Lineage B.1.617.2; Delta variant) was obtained through BEI Resources, NIAID, NIH: NR-55672. Virus was propagated on Vero-TMPRSS2 and quantified on Vero cells. Gene sequence of the spike glycoprotein was carried out to confirm exact genetic identity to the original isolate. Passage 2 virus stocks were used in this in vivo hamster study. All infectious work was carried out under ABSL-3 conditions at the NRC. Animals were randomly allocated into thirteen different experimental groups (n=6 animals per group) and were challenged with Delta variant at 8×10³ PFU/animal. The test articles, i.e., the decavalent IgM-based construct v36708 and the bivalent IgG-based construct v36710, were administered intranasally at 6 hr post-challenge at 0.1, 1, or 10 mg/kg dosed in 50 μL per nares. Daily weight and clinical scores were determined. Oral swabs were taken dally starting on day 2 to 5 dpi and transferred directly to 600 μl RNA/DNA Shield (Zymo Research, Irvine, CA). Lung tissue for viral RNA (vRNA) isolation was collected directly into 1 mL RNA/DNA Shield in homogenizer tubes. Viral RNA was extracted under BSL-3 conditions from oral swab samples or mechanically homogenized lung tissues using Quick-RNA Viral Kit (Zymo Research, Irvine, CA).

Viral genomic RNA was quantified by real time-PCR to the target viral envelope gene as previous described. Five days after infection, bronchoalveolar lavage (BAL) and lung tissue necropsy were collected for measurement of infectious viral particles by plaque assay. qRT-PCR was also performed to measure viral RNA in the samples using known methods. Plaque assay was carried out as previous described. In brief, centrifuge clarified supernatant from homogenized lung tissues were diluted in a 1 in 10 serial dilution in infection media. Virus was adsorbed on Vero cells for 1 h at 37° C. before inoculum was removed and overlaid with infection media containing 0.6% ultrapure, low-melting point agarose. Infected cells were incubated at 37° C./5% CO₂ for 72 h. After incubation, cells were fixed with 10% formaldehyde and stained with crystal violet. Plaques were enumerated and PFU was determined per gram of lung tissue or per mL of BAL.

### Results

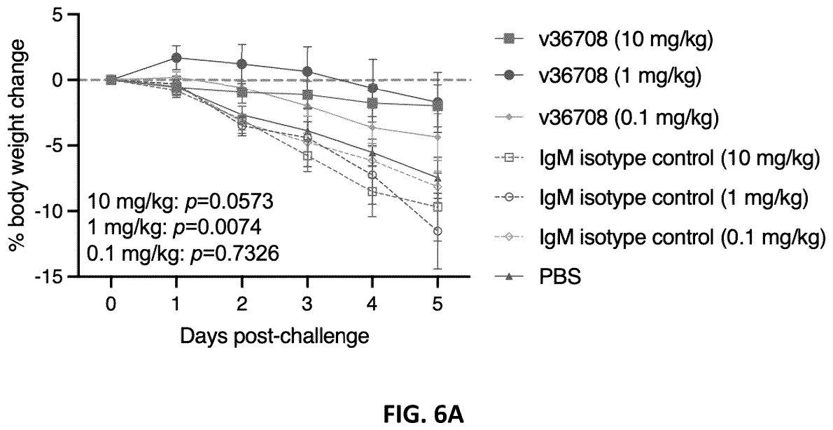

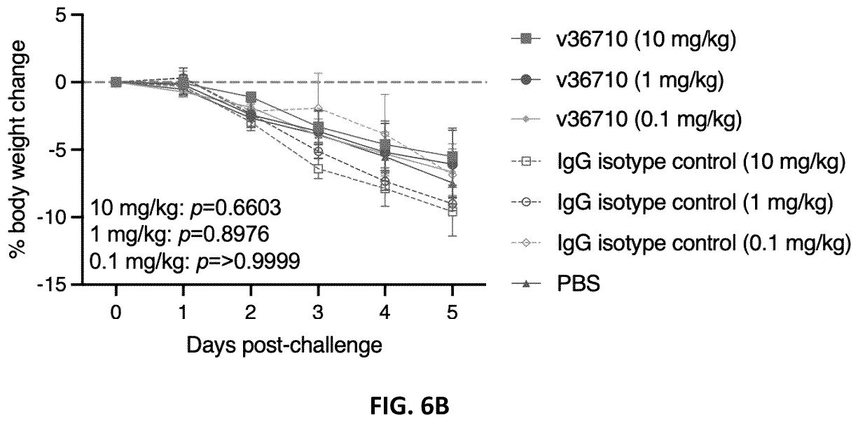

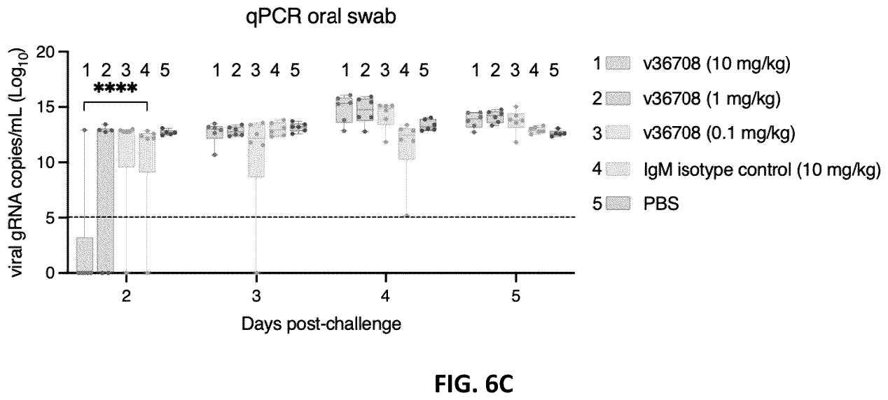

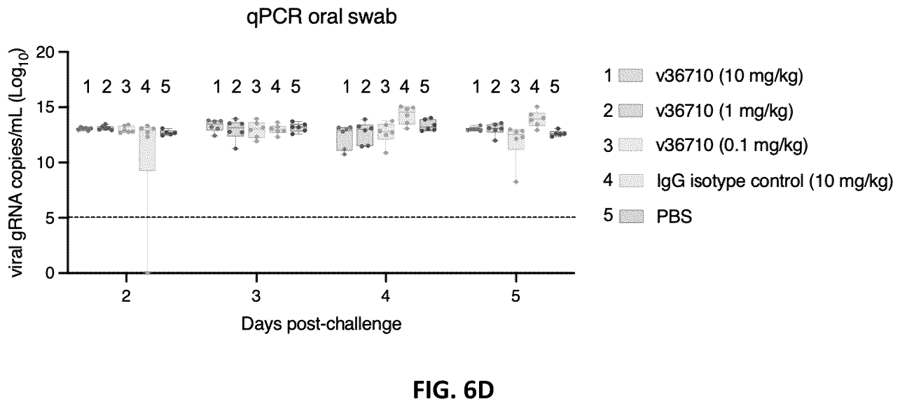

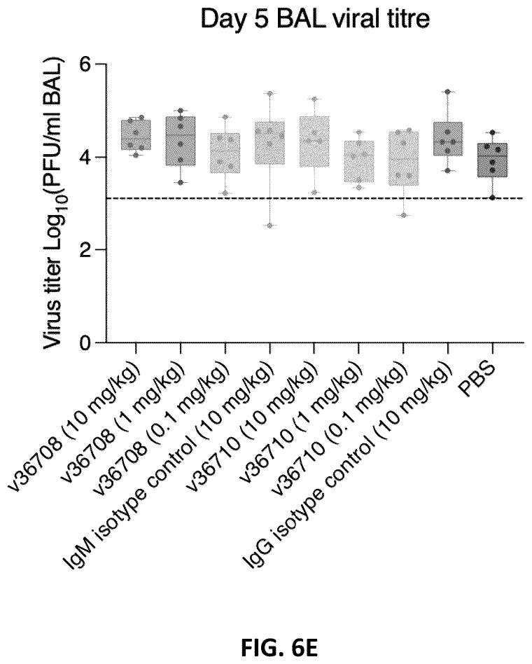

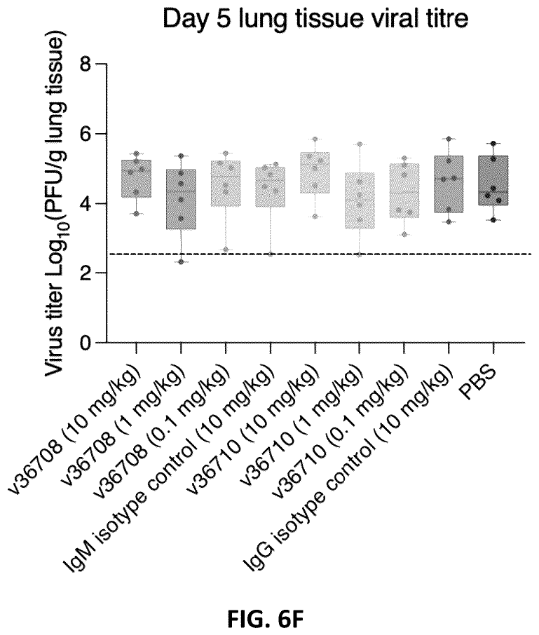

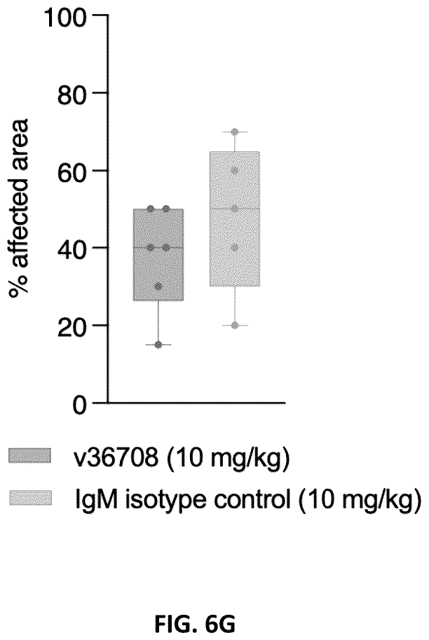

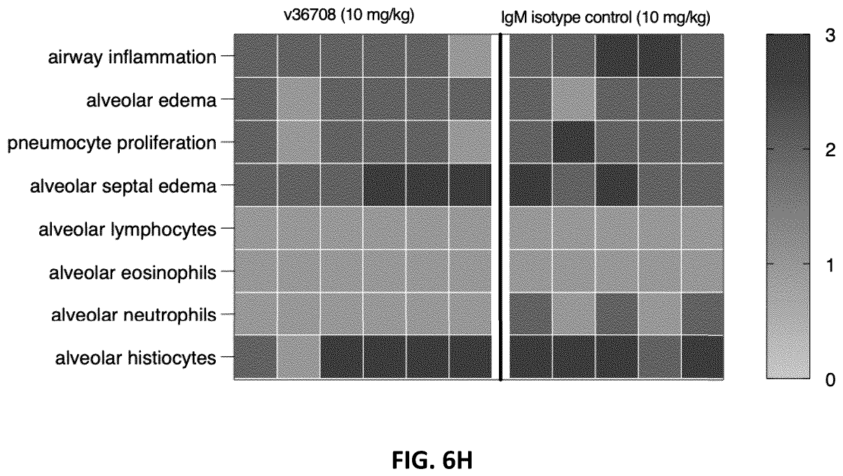

The decavalent IgM-based ACE2 decoy construct showed the greatest protection in challenged animals. Infected hamsters therapeutically treated with IgM-based ACE2 decoy constructs at 1 mg/kg showed weight gain within the first 72 hrs, while treatment with 10 mg/kg resulted in less than 2% weight change over 5 days post-infection (see, e.g., FIG. 6A). Contrarily, the IgM isotype (10 mg/kg) and PBS controls resulted in over a loss of 10% and 7%, respectively, in weight change by 5 dpi. This protection was accompanied with a reduction in viral genomic RNA in oral swabs taken on day 2 post viral challenge (see, e.g., FIG. 6C). However, hamster weight started to decline in all treatment groups by days 4 and 5 post injection, with an accompanying increase in viral burden in both lung tissues and oral swabs (see, e.g., FIGS. 6C-6F). Live virus quantified from the lung tissue and bronchoalveolar lavage (BAL) at day 5 post injection showed little difference to the controls (see, e.g., FIGS. 6E-6H). A positive weight change can be inversely correlated with a reduction in viral burden. This indicates that while the decavalent IgM-based ACE2 decoy construct is effective in reducing both weight loss and viral burden within the first 3 days after treatment, a single administration may not have been sufficient enough to completely nullify the infection.

In contrast to the IgM-based construct, the tested monovalent IgG-based ACE2 decoy construct dosed equivalently did not afford the same protection as the decavalent IgM-based construct based on prevention of hamster weight loss at equivalent 1 and 10 mg/kg doses tested (see, e.g., FIGS. 6A-6B).

Taken together, these findings further demonstrate that the ACE2 multivalency afforded by, e.g., the decavalent IgM-based scaffolds as used in, e.g., the construct v36708, can further increase the protective efficacy compared to mono-valent constructs. Furthermore, and as viral replication and lung pathology can change very rapidly in the hamster model, demonstrating therapeutic efficacy using a single treatment post-challenge was chosen for this study. The results demonstrate that a single treatment with a decavalent IgM-based ACE2 decoy construct at doses of 1 and 10 mg/kg can, at least temporarily, reduce viral load in vivo.

TABLE 10
SARS-CoV-2 Variant Nomenclature

| | | | | |
|---|---|---|---|---|
| | Pango | GISAID | Nextstrain | RBD |
| WHO label | lineage | clade | clade | mutations |

Exemplary variants of concern to public health:

| | | | | |
|---|---|---|---|---|
| Alpha | B.1.1.7 | GRY | 20I (V1) | N501Y |
| Beta | B.1.351 | GRY/501Y.V2 | 20H (V2) | K417N, E484K, N501Y |
| Gamma | P.1 | GR/501Y.V3 | 20J (V3) | K417T, E484K, N501Y |
| Delta | B.1.617.2 | G/478K.V1 | 21A | L452R, T478K |
| Omicron | B.1.1.529 | GR/484A | 21K, 21L, | G339D, S371L, S373P, |
| | | | 21M, 22A, | S375F, K417N, N440K, |
| | | | 22B, 22C, | G446S, S477N, T478K, |
| | | | 22D | E484A, Q439R, G496S, |
| | | | | Q498R, N501Y, Y505H |

Exemplary variants of Interest:

| | | | | |
|---|---|---|---|---|
| Eta | B.1.525 | G/484K.V3 | 21D | E484K |
| Iota | B.1.526 | GH/253G.V1 | 21F | E484K |
| Kappa | B.1.617.1 | G/452R.V3 | 21B | L452R, E484Q |
| Lambda | C.37 | GR/452Q.V1 | 21G | L452Q, F490S |
| Mu | B.1.621 | GH | 21H | R346K, E484K, N501Y |

Additional variants:

| | | | | |
|---|---|---|---|---|
| Epsilon | B.1.427, B.1.429 | GH/452R.V1 | 21C | L452R |
| Theta | P.3 | GR | 21E | E484K, N501Y |
| Zeta | P.2 | GR | 20B/S.484K | E484K |

TABLE 11
SEQUENCE TABLE

| Variant No./ | SEQ ID | |
|---|---|---|
| Description | NO: | SEQUENCE |
| Human WT | 1 | MSSSSWLLLSLVAVTAAQSTIEEQAKTFLDKFNHEAEDLFYQS |
| ACE2 protein | | SLASWNYNTNITEENVQNMNNAGDKWSAFLKEQSTLAQMY |
| | | PLQEIQNLTVKLQLQALQQNGSSVLSEDKSKRLNTILNTMSTI |
| | | YSTGKVCNPDNPQECLLLEPGLNEIMANSLDYNERLWAWES |
| | | WRSEVGKQLRPLYEEYVVLKNEMARANHYEDYGDYWRGD |
| | | YEVNGVDGYDYSRGQLIEDVEHTFEEIKPLYEHLHAYVRAKL |
| | | MNAYPSYISPIGCLPAHLLGDMWGRFWTNLYSLTVPFGQKPN |
| | | IDVTDAMVDQAWDAQRIFKEAEKFFVSVGLPNMTQGFWENS |
| | | MLTDPGNVQKAVCHPTAWDLGKGDFRILMCTKVTMDDFLT |
| | | AHHEMGHIQYDMAYAAQPFLLRNGANEGFHEAVGEIMSLSA |
| | | ATPKHLKSIGLLSPDFQEDNETEINFLLKQALTIVGTLPFTYML |
| | | EKWRWMVFKGEIPKDQWMKKWWEMKREIVGVVEPVPHDE |
| | | TYCDPASLFHVSNDYSFIRYYTRTLYQFQFQEALCQAAKHEG |
| | | PLHKCDISNSTEAGQKLFNMLRLGKSEPWTLALENVVGAKN |
| | | MNVRPLLNYFEPLFTWLKDQNKNSFVGWSTDWSPYADQSIK |
| | | VRISLKSALGDKAYEWNDNEMYLFRSSVAYAMRQYFLKVKN |
| | | QMILFGEEDVRVANLKPRISFNFFVTAPKNVSDIIPRTEVEKAI |
| | | RMSRSRINDAFRLNDNSLEFLGIQPTLGPPNQPPVSIWLIVFGV |
| | | VMGVIVVGIVILIFTGIRDRKKKNKARSGENPYASIDISKGENN |
| | | PGFQNTDDVQTSF |
| Q18-S740 of | 2 | QSTIEEQAKTFLDKFNHEAEDLFYQSSLASWNYNTNITEENVQ |
| Human WT | | NMNNAGDKWSAFLKEQSTLAQMYPLQEIQNLTVKLQLQALQ |
| ACE2 protein | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHHEMGHIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| Q18-D615 of | 3 | QSTIEEQAKTFLDKFNHEAEDLFYQSSLASWNYNTNITEENVQ |
| Human WT | | NMNNAGDKWSAFLKEQSTLAQMYPLQEIQNLTVKLQLQALQ |
| ACE2 protein | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHHEMGHIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYAD |
| “KO” variant | 4 | QSTIEEQAKTFLDKFNHEAEDLFYQSSLASWNYNTNITEENVQ |
| of Q18-S740 | | NMNNAGDKWSAFLKEQSTLAQMYPLQEIQNLTVKLQLQALQ |
| of human | | QNGSSVLSEDKSKRLNTILNTMSTIYSTGKVCNPDNPQECLLL |
| ACE2 | | EPGLNEIMANSLDYNERLWAWESWRSEVGKQLRPLYEEYVV |
| (H374N- | | LKNEMARANHYEDYGDYWRGDYEVNGVDGYDYSRGQLIED |
| H378N) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 44 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| A396C_A569C) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGCNEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLCLENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 45 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| H373C_A412C) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTACNEMGNIQYDMAYAAQ |
| | | PFLLRNGANEGFHEAVGEIMSLSCATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 46 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| A533C_A550C) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQACKHEGPLHKCDISNSTECGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 47 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| A311C_A372C) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KECEKFFVSVGLPNMTQGFWENSMLTDPGNVQKAVCHPTA |
| | | WDLGKGDFRILMCTKVTMDDFLTCHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 48 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| A304C_T362C) | | VEHTFEEIKPLYEHLHAYVRAKLMNAYPSYISPIGCLPAHLLG |
| | | DMWGRFWTNLYSLTVPFGQKPNIDVTDAMVDQAWDCQRIF |
| | | KEAEKFFVSVGLPNMTQGFWENSMLTDPGNVQKAVCHPTA |
| | | WDLGKGDFRILMCCKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 49 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| A650C_S707C) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYCMRQYFLKVKNQMILFGEEDVRVANLKPRIS |
| | | FNFFVTAPKNVSDIIPRTEVEKAIRMCRSRINDAFRLNDNSLEF |
| | | LGIQPTLGPPNQPPVS |
| “KO” and “S” | 50 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| V293C_L423C) | | VEHTFEEIKPLYEHLHAYVRAKLMNAYPSYISPIGCLPAHLLG |
| | | DMWGRFWTNLYSLTVPFGQKPNIDCTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGCLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 51 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| L520I) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTIYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 52 | QSTIEEQAKTFLDKFNHEAEDLFYQSSLASWNYNTNITEENVQ |
| variant of | | NMNNAGDKWSAFYKEQSTLAQMYPLQEIQNLTVKLQLQAL |
| Q18-S740 of | | QQNGSSVLSEDKSKRLNTILNTMSTIYSTGKVCNPDNPQECLL |
| human ACE2 | | LEPGLNEIMANSLDYNERLWAWESWRSEVGKQLRPLYEEYV |
| (H374N_H378N_ | | VLKNEMARANHYEDYGDYWRGDYEVNGVDGYDYSRGQLIE |
| L73Y) | | DVEHTFEEIKPLYEHLHAYVRAKLMNAYPSYISPIGCLPAHLL |
| | | GDMWGRFWTNLYSLTVPFGQKPNIDVTDAMVDQAWDAQRI |
| | | FKEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 53 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| A342V) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKVVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 54 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| V447I) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIIGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 55 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| V226I) | | IEHTFEEIKPLYEHLHAYVRAKLMNAYPSYISPIGCLPAHLLGD |
| | | MWGRFWTNLYSLTVPFGQKPNIDVTDAMVDQAWDAQRIFK |
| | | EAEKFFVSVGLPNMTQGFWENSMLTDPGNVQKAVCHPTAW |
| | | DLGKGDFRILMCTKVTMDDFLTAHNEMGNIQYDMAYAAQPF |
| | | LLRNGANEGFHEAVGEIMSLSAATPKHLKSIGLLSPDFQEDNE |
| | | TEINFLLKQALTIVGTLPFTYMLEKWRWMVFKGEIPKDQWM |
| | | KKWWEMKREIVGVVEPVPHDETYCDPASLFHVSNDYSFIRY |
| | | YTRTLYQFQFQEALCQAAKHEGPLHKCDISNSTEAGQKLFNM |
| | | LRLGKSEPWTLALENVVGAKNMNVRPLLNYFEPLFTWLKDQ |
| | | NKNSFVGWSTDWSPYADQSIKVRISLKSALGDKAYEWNDNE |
| | | MYLFRSSVAYAMRQYFLKVKNQMILFGEEDVRVANLKPRISF |
| | | NFFVTAPKNVSDIIPRTEVEKAIRMSRSRINDAFRLNDNSLEFL |
| | | GIQPTLGPPNQPPVS |
| “KO” and “S” | 56 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| Q526F) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFFEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 57 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| Q526Y) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFYEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 58 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| H417F) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKFLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 59 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| T362I) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCIKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 60 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| T453L) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFLYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 61 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| V647I) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSIAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO″ and “S” | 62 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| S507L) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVLNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 63 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| S411H) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLHAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 64 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| M360W) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILWCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 65 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| M360F) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILFCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 66 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| Q522M) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYMFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 67 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLWEEYVV |
| (H374N_H378N_ | | LKNEMARANHYEDYGDYWRGDYEVNGVDGYDYSRGQLIED |
| Y180W) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 68 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| S411I) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLIAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 69 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| Q526H) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFHEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| “KO” and “S” | 71 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| T686I) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVIAPKNVSDIIPRTEVEKAIRMSRSRINDAFRLNDNSLEFL |
| | | GIQPTLGPPNQPPVS |
| “KO” and “S” | 72 | QSTIEEQAKTFLDKFNHEAEDLFYQSSLASWNYNTNITEENVQ |
| variant of | | NMNNAGDKWSAFLKEQSTLAQMYPLQEIQNLTVKLQLQALQ |
| Q18-S740 of | | QNGSSVLSEDKSKRLNTILNTMSTIYSTGKVCNPDNPQECLLL |
| human ACE2 | | EPGLNEIMANSLDYNERLWAWESWRSEVGKQLRPLYEEYVV |
| (H374N_H378N_ | | LKNEMARANHYEDYGDYWRGDYEVNGVDGYDYSRGQLIED |
| V647L) | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSLAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVS |
| IgM CH2 | 5 | VIAELPPKVSVFVPPRDGFFGNPRKSKLICQATGFSPRQIQVSW |
| domain | | LREGKQVGSGVTTDQVQAEAKESGPTTYKVTSTLTIKESDWL |
| | | SQSMFTCRVDHRGLTFQQNASSMCVP |
| IgM CH3 | 6 | DQDTAIRVFAIPPSFASIFLTKSTKLTCLVTDLTTYDSVTISWTR |
| domain | | QNGEAVKTHTNISESHPNATFSAVGEASICEDDWNSGERFTCT |
| | | VTHTDLPSPLKQTISRPK |
| IgM CH4 | 7 | GVALHRPDVYLLPPAREQLNLRESATITCLVTGFSPADVFVQ |
| domain | | WMQRGQPLSPEKYVTSAPMPEPQAPGRYFAHSILTVSEEEWN |
| | | TGETYTCVVAHEALPNRVTERTVDKSTG |
| IgM CH4 | 8 | GVALHRPDVYLLPPAREQLNLRESATITCLVTGFSPADVFVQ |
| domain (incl. | | WMQRGQPLSPEKYVTSAPMPEPQAPGRYFAHSILTVSEEEWN |
| tailpiece | | TGETYTCVVAHEALPNRVTERTVDKSTGKPTLYNVSLVMSDT |
| sequence) | | AGTCY |
| IgM CH4 | 9 | KPTLYNVSLVMSDTAGTCY |
| tailpiece | | |
| sequence | | |
| 26045 | 10 | QEDERIVLVDNKCKCARITSRIIRSSEDPNEDIVERNIRIIVPLNN |
| J-Chain | | RENISDPTSPLRTRFVYHLSDLCKKCDPTEVELDNQIVTATQS |
| | | NICDEDSATETCYTYDRNKCYTAVVPLVYGGETKMVETALTP |
| | | DACYPDGGGGSEQKLISEEDL |
| | 11 | CAGGAGGACGAGAGGATCGTGCTGGTGGATAACAAGTGCA |
| | | AGTGTGCCCGGATCACATCTAGGATCATCCGCAGCTCCGA |
| | | GGACCCTAACGAGGATATCGTGGAGCGGAATATCAGAATC |
| | | ATCGTGCCACTGAACAATAGAGAGAATATCTCTGACCCCA |
| | | CCAGCCCTCTGCGGACAAGATTCGTGTACCACCTGTCCGAC |
| | | CTGTGCAAGAAGTGTGATCCAACCGAGGTGGAGCTGGACA |
| | | ACCAGATCGTGACCGCCACACAGAGCAATATCTGCGACGA |
| | | GGATTCCGCCACCGAGACATGCTACACCTATGATCGGAAC |
| | | AAGTGTTACACAGCCGTGGTGCCCCTGGTGTATGGAGGAG |
| | | AGACAAAGATGGTGGAGACCGCCCTGACACCAGACGCATG |
| | | TTATCCAGATGGAGGAGGAGGCTCCGAGCAGAAGCTGATC |
| | | TCTGAGGAGGATCTG |
| 23742 | 12 | QEDERIVLVDNKCKCARITSRIIRSSEDPNEDIVERNIRIIVPLNN |
| J-Chain | | RENISDPTSPLRTRFVYHLSDLCKKCDPTEVELDNQIVTATOS |
| | | NICDEDSATETCYTYDRNKCYTAVVPLVYGGETKMVETALTP |
| | | DACYPD |
| | 13 | CAGGAGGACGAGAGGATCGTGCTGGTGGATAACAAGTGCA |
| | | AGTGTGCCCGGATCACAAGCAGGATCATCCGCAGCTCCGA |
| | | GGACCCTAACGAGGATATCGTGGAGCGGAATATCAGAATC |
| | | ATCGTGCCACTGAACAATAGAGAGAATATCTCTGACCCCA |
| | | CCAGCCCTCTGCGGACAAGATTCGTGTACCACCTGTCCGAC |
| | | CTGTGCAAGAAGTGTGATCCAACCGAGGTGGAGCTGGACA |
| | | ACCAGATCGTGACCGCCACACAGTCCAATATCTGCGACGA |
| | | GGATTCTGCCACCGAGACATGCTACACCTATGATCGGAAC |
| | | AAGTGTTACACAGCCGTGGTGCCCCTGGTGTATGGAGGAG |
| | | AGACAAAGATGGTGGAGACCGCCCTGACACCAGACGCCTG |
| | | TTATCCCGAT |
| 26007 | 14 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHHEMGHIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSEPKSSDKTHTCPPCPAPEAAGGPSVFLF |
| | | PPKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYVYPPSRDELTKNQVSLTCL |
| | | VKGFYPSDIAVEWESNGQPENNYKTTPPVLDSDGSFALVSKL |
| | | TVDKSRWQQGNVFSCSVMHEALHNHYTQKSLSLSPGGGGGS |
| | | EQKLISEEDL |
| | 15 | CAGTCCACAATCGAGGAGCAGGCCAAGACCTTCCTGGACA |
| | | AGTTTAACCACGAGGCCGAGGATCTGTTCTATCAGAGCTCC |
| | | CTGGCCAGCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGTCTGAGGAT |
| | | AAGAGCAAGCGGCTGAATACAATCCTGAACACCATGTCCA |
| | | CAATCTATTCTACCGGCAAGGTGTGCAATCCCGACAACCCT |
| | | CAGGAGTGTCTGCTGCTGGAGCCTGGCCTGAATGAGATCA |
| | | TGGCCAACAGCCTGGATTACAATGAGAGGCTGTGGGCATG |
| | | GGAGAGCTGGCGCTCCGAAGTGGGCAAGCAGCTGCGGCCA |
| | | CTGTACGAGGAGTATGTGGTGCTGAAGAACGAGATGGCCA |
| | | GAGCCAATCACTACGAGGACTATGGCGATTACTGGAGGGG |
| | | CGACTACGAGGTGAACGGCGTGGACGGCTACGATTATTCC |
| | | AGAGGCCAGCTGATCGAGGATGTGGAGCACACATTCGAGG |
| | | AGATCAAGCCACTGTATGAGCACCTGCACGCCTACGTGAG |
| | | GGCCAAGCTGATGAACGCCTATCCTTCTTACATCAGCCCAA |
| | | TCGGATGCCTGCCCGCCCACCTGCTGGGCGACATGTGGGG |
| | | CCGCTTCTGGACAAACCTGTACAGCCTGACCGTGCCTTTTG |
| | | GCCAGAAGCCAAATATCGACGTGACCGATGCCATGGTGGA |
| | | CCAGGCCTGGGATGCCCAGCGGATCTTCAAGGAGGCCGAG |
| | | AAGTTCTTCGTGAGCGTGGGCCTGCCAAACATGACACAGG |
| | | GCTTTTGGGAGAACAGCATGCTGACCGACCCCGGCAACGT |
| | | GCAGAAGGCCGTGTGCCACCCTACAGCCTGGGACCTGGGC |
| | | AAGGGCGATTTCAGAATCCTGATGTGCACCAAGGTGACAA |
| | | TGGACGATTTTCTGACCGCCCACCACGAGATGGGCCACATC |
| | | CAGTATGATATGGCATACGCAGCACAGCCCTTCCTGCTGAG |
| | | GAATGGAGCCAACGAGGGCTTTCACGAGGCCGTGGGCGAG |
| | | ATCATGTCTCTGAGCGCCGCCACACCAAAGCACCTGAAGT |
| | | CCATCGGCCTGCTGTCTCCCGACTTCCAGGAGGATAACGAG |
| | | ACCGAGATCAATTTTCTGCTGAAGCAGGCCCTGACCATCGT |
| | | GGGCACACTGCCCTTCACCTATATGCTGGAGAAGTGGAGA |
| | | TGGATGGTGTTTAAGGGCGAGATCCCTAAGGACCAGTGGA |
| | | TGAAGAAGTGGTGGGAGATGAAGAGGGAGATCGTGGGAG |
| | | TGGTGGAGCCCGTGCCTCACGACGAGACCTACTGTGATCCC |
| | | GCCTCCCTGTTCCACGTGAGCAACGACTATTCCTTTATCAG |
| | | GTACTATACCCGCACACTGTACCAGTTCCAGTTTCAGGAGG |
| | | CCCTGTGCCAGGCAGCAAAGCACGAGGGCCCACTGCACAA |
| | | GTGTGATATCTCCAACTCTACAGAGGCCGGACAGAAGCTG |
| | | TTCAATATGCTGCGCCTGGGCAAGAGCGAGCCATGGACCC |
| | | TGGCCCTGGAGAACGTGGTGGGAGCCAAGAATATGAACGT |
| | | GCGGCCCCTGCTGAATTATTTCGAGCCTCTGTTTACATGGC |
| | | TGAAGGATCAGAATAAGAACAGCTTTGTGGGCTGGTCTAC |
| | | CGACTGGAGCCCCTACGCCGATCAGTCCATCAAGGTGAGA |
| | | ATCTCCCTGAAGTCTGCCCTGGGCGACAAGGCCTATGAGTG |
| | | GAATGATAACGAGATGTACCTGTTCAGATCCTCTGTGGCCT |
| | | ATGCCATGAGGCAGTACTTCCTGAAGGTGAAGAACCAGAT |
| | | GATCCTGTTTGGCGAGGAGGACGTGCGGGTGGCCAATCTG |
| | | AAGCCTAGAATCAGCTTTAACTTCTTTGTGACAGCCCCAAA |
| | | GAACGTGAGCGATATCATCCCCAGAACCGAGGTGGAGAAG |
| | | GCCATCCGGATGAGCCGGTCCAGAATCAACGACGCCTTCC |
| | | GCCTGAATGATAACTCCCTGGAGTTTCTGGGCATCCAGCCT |
| | | ACCCTGGGCCCACCTAATCAGCCACCCGTGAGCGAGCCAA |
| | | AGAGCTCCGACAAGACCCACACATGCCCTCCATGTCCGGC |
| | | GCCAGAGGCCGCCGGAGGACCTAGCGTGTTCCTGTTTCCCC |
| | | CTAAGCCAAAGGATACACTGATGATCAGCCGGACACCAGA |
| | | GGTGACCTGCGTGGTGGTGTCTGTGAGCCACGAGGACCCC |
| | | GAGGTGAAGTTCAACTGGTATGTGGATGGCGTGGAGGTGC |
| | | ACAATGCCAAGACCAAGCCTAGAGAGGAGCAGTATAACAG |
| | | CACATACAGGGTGGTGTCCGTGCTGACCGTGCTGCACCAG |
| | | GACTGGCTGAACGGCAAGGAGTACAAGTGCAAGGTGTCTA |
| | | ATAAGGCCCTGCCTGCCCCAATCGAGAAGACCATCAGCAA |
| | | GGCAAAGGGACAGCCTCGCGAACCACAGGTGTACGTGTAT |
| | | CCACCCTCCCGGGACGAGCTGACAAAGAACCAGGTGTCTC |
| | | TGACCTGTCTGGTGAAGGGCTTTTATCCCTCTGATATCGCC |
| | | GTGGAGTGGGAGAGCAATGGCCAGCCTGAGAACAATTACA |
| | | AGACCACACCTCCAGTGCTGGACAGCGATGGCTCCTTCGCC |
| | | CTGGTGAGCAAGCTGACAGTGGACAAGTCCAGGTGGCAGC |
| | | AGGGCAACGTGTTTTCCTGTTCTGTGATGCACGAGGCCCTG |
| | | CACAATCACTACACCCAGAAGAGCCTGAGCTTAAGCCCAG |
| | | GAGGAGGAGGAGGCTCTGAGCAGAAGCTGATCAGCGAGG |
| | | AGGATCTG |
| 12153 | 16 | EPKSSDKTHTCPPCPAPEAAGGPSVFLFPPKPKDTLMISRTPEV |
| | | TCVVVSVSHEDPEVKFNWYVDGVEVHNAKTKPREEQYNSTY |
| | | RVVSVLTVLHQDWLNGKEYKCKVSNKALPAPIEKTISKAKGQ |
| | | PREPQVYVLPPSRDELTKNQVSLLCLVKGFYPSDIAVEWESNG |
| | | QPENNYLTWPPVLDSDGSFFLYSKLTVDKSRWQQGNVFSCSV |
| | | MHEALHNHYTQKSLSLSPG |
| | 17 | GAGCCAAAGAGCTCCGACAAGACCCACACATGCCCCCCTT |
| | | GTCCGGCGCCAGAGGCAGCAGGAGGACCAAGCGTGTTCCT |
| | | GTTTCCACCCAAGCCCAAAGACACCCTGATGATTAGCCGA |
| | | ACCCCTGAAGTCACATGCGTGGTCGTGTCCGTGTCTCACGA |
| | | GGACCCAGAAGTCAAGTTCAACTGGTACGTGGATGGCGTC |
| | | GAGGTGCATAATGCCAAGACAAAACCCCGGGAGGAACAGT |
| | | ACAACAGCACCTATAGAGTCGTGTCCGTCCTGACAGTGCTG |
| | | CACCAGGATTGGCTGAACGGCAAGGAATATAAGTGCAAAG |
| | | TGTCCAATAAGGCCCTGCCCGCTCCTATCGAGAAAACCATT |
| | | TCTAAGGCAAAAGGCCAGCCTCGCGAACCACAGGTCTACG |
| | | TGCTGCCTCCATCCCGGGACGAGCTGACAAAGAACCAGGT |
| | | CTCTCTGCTGTGCCTGGTGAAAGGCTTCTATCCATCAGATA |
| | | TTGCTGTGGAGTGGGAAAGCAATGGGCAGCCCGAGAACAA |
| | | TTACCTGACTTGGCCCCCTGTGCTGGACTCTGATGGGAGTT |
| | | TCTTTCTGTATTCTAAGCTGACCGTGGATAAAAGTAGGTGG |
| | | CAGCAGGGAAATGTCTTTAGTTGTTCAGTGATGCATGAAGC |
| | | CCTGCATAACCACTACACCCAGAAAAGCCTGTCCCTGTCCC |
| | | CCGGA |
| 26011 | 18 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSEPKSSDKTHTCPPCPAPEAAGGPSVFLF |
| | | PPKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYVYPPSRDELTKNQVSLTCL |
| | | VKGFYPSDIAVEWESNGQPENNYKTTPPVLDSDGSFALVSKL |
| | | TVDKSRWQQGNVFSCSVMHEALHNHYTQKSLSLSPGGGGGS |
| | | EQKLISEEDL |
| | 19 | CAGTCCACAATCGAGGAGCAGGCCAAGACCTTCCTGGACA |
| | | AGTTTAACCACGAGGCCGAGGATCTGTTCTATCAGAGCTCC |
| | | CTGGCCAGCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGTCTGAGGAT |
| | | AAGAGCAAGCGGCTGAATACAATCCTGAACACCATGTCCA |
| | | CAATCTATTCTACCGGCAAGGTGTGCAATCCCGACAACCCT |
| | | CAGGAGTGTCTGCTGCTGGAGCCTGGCCTGAATGAGATCA |
| | | TGGCCAACAGCCTGGATTACAATGAGAGGCTGTGGGCATG |
| | | GGAGAGCTGGCGCTCCGAAGTGGGCAAGCAGCTGCGGCCA |
| | | CTGTACGAGGAGTATGTGGTGCTGAAGAACGAGATGGCCA |
| | | GAGCCAATCACTACGAGGACTATGGCGATTACTGGAGGGG |
| | | CGACTACGAGGTGAACGGCGTGGACGGCTACGATTATTCC |
| | | AGAGGCCAGCTGATCGAGGATGTGGAGCACACATTCGAGG |
| | | AGATCAAGCCACTGTATGAGCACCTGCACGCCTACGTGAG |
| | | GGCCAAGCTGATGAACGCCTATCCTTCTTACATCAGCCCAA |
| | | TCGGATGCCTGCCCGCCCACCTGCTGGGCGACATGTGGGG |
| | | CCGCTTCTGGACAAACCTGTACAGCCTGACCGTGCCTTTTG |
| | | GCCAGAAGCCAAATATCGACGTGACCGATGCCATGGTGGA |
| | | CCAGGCCTGGGATGCCCAGCGGATCTTCAAGGAGGCCGAG |
| | | AAGTTCTTCGTGAGCGTGGGCCTGCCAAACATGACACAGG |
| | | GCTTTTGGGAGAACAGCATGCTGACCGACCCCGGCAACGT |
| | | GCAGAAGGCCGTGTGCCACCCTACAGCCTGGGACCTGGGC |
| | | AAGGGCGATTTCAGAATCCTGATGTGCACCAAGGTGACAA |
| | | TGGACGATTTTCTGACCGCCCACAACGAGATGGGCAACAT |
| | | CCAGTATGATATGGCATACGCAGCACAGCCCTTCCTGCTGA |
| | | GGAATGGAGCCAACGAGGGCTTTCACGAGGCCGTGGGCGA |
| | | GATCATGTCTCTGAGCGCCGCCACACCAAAGCACCTGAAG |
| | | TCCATCGGCCTGCTGTCTCCCGACTTCCAGGAGGATAACGA |
| | | GACCGAGATCAATTTTCTGCTGAAGCAGGCCCTGACCATCG |
| | | TGGGCACACTGCCCTTCACCTATATGCTGGAGAAGTGGAG |
| | | ATGGATGGTGTTTAAGGGCGAGATCCCTAAGGACCAGTGG |
| | | ATGAAGAAGTGGTGGGAGATGAAGAGGGAGATCGTGGGA |
| | | GTGGTGGAGCCCGTGCCTCACGACGAGACCTACTGTGATC |
| | | CCGCCTCCCTGTTCCACGTGAGCAACGACTATTCCTTTATC |
| | | AGGTACTATACCCGCACACTGTACCAGTTCCAGTTTCAGGA |
| | | GGCCCTGTGCCAGGCAGCAAAGCACGAGGGCCCACTGCAC |
| | | AAGTGTGATATCTCCAACTCTACAGAGGCCGGACAGAAGC |
| | | TGTTCAATATGCTGCGCCTGGGCAAGAGCGAGCCATGGAC |
| | | CCTGGCCCTGGAGAACGTGGTGGGAGCCAAGAATATGAAC |
| | | GTGCGGCCCCTGCTGAATTATTTCGAGCCTCTGTTTACATG |
| | | GCTGAAGGATCAGAATAAGAACAGCTTTGTGGGCTGGTCT |
| | | ACCGACTGGAGCCCCTACGCCGATCAGTCCATCAAGGTGA |
| | | GAATCTCCCTGAAGTCTGCCCTGGGCGACAAGGCCTATGA |
| | | GTGGAATGATAACGAGATGTACCTGTTCAGATCCTCTGTGG |
| | | CCTATGCCATGAGGCAGTACTTCCTGAAGGTGAAGAACCA |
| | | GATGATCCTGTTTGGCGAGGAGGACGTGCGGGTGGCCAAT |
| | | CTGAAGCCTAGAATCAGCTTTAACTTCTTTGTGACAGCCCC |
| | | AAAGAACGTGAGCGATATCATCCCCAGAACCGAGGTGGAG |
| | | AAGGCCATCCGGATGAGCCGGTCCAGAATCAACGACGCCT |
| | | TCCGCCTGAATGATAACTCCCTGGAGTTTCTGGGCATCCAG |
| | | CCTACCCTGGGCCCACCTAATCAGCCACCCGTGAGCGAGC |
| | | CAAAGAGCTCCGACAAGACCCACACATGCCCTCCATGTCC |
| | | GGCGCCAGAGGCCGCCGGAGGACCTAGCGTGTTCCTGTTT |
| | | CCCCCTAAGCCAAAGGATACACTGATGATCAGCCGGACAC |
| | | CAGAGGTGACCTGCGTGGTGGTGTCTGTGAGCCACGAGGA |
| | | CCCCGAGGTGAAGTTCAACTGGTATGTGGATGGCGTGGAG |
| | | GTGCACAATGCCAAGACCAAGCCTAGAGAGGAGCAGTATA |
| | | ACAGCACATACAGGGTGGTGTCCGTGCTGACCGTGCTGCA |
| | | CCAGGACTGGCTGAACGGCAAGGAGTACAAGTGCAAGGTG |
| | | TCTAATAAGGCCCTGCCTGCCCCAATCGAGAAGACCATCA |
| | | GCAAGGCAAAGGGACAGCCTCGCGAACCACAGGTGTACGT |
| | | GTATCCACCCTCCCGGGACGAGCTGACAAAGAACCAGGTG |
| | | TCTCTGACCTGTCTGGTGAAGGGCTTTTATCCCTCTGATATC |
| | | GCCGTGGAGTGGGAGAGCAATGGCCAGCCTGAGAACAATT |
| | | ACAAGACCACACCTCCAGTGCTGGACAGCGATGGCTCCTT |
| | | CGCCCTGGTGAGCAAGCTGACAGTGGACAAGTCCAGGTGG |
| | | CAGCAGGGCAACGTGTTTTCCTGTTCTGTGATGCACGAGGC |
| | | CCTGCACAATCACTACACCCAGAAGAGCCTGAGCTTAAGC |
| | | CCAGGAGGAGGAGGAGGCTCTGAGCAGAAGCTGATCAGC |
| | | GAGGAGGATCTG |
| 26008 | 20 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHHEMGHIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSEPKSSDKTHTCPPCPAPEAAGGPSVFLF |
| | | PPKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYVLPPSRDELTKNQVSLLCL |
| | | VKGFYPSDIAVEWESNGQPENNYLTWPPVLDSDGSFFLYSKL |
| | | TVDKSRWQQGNVFSCSVMHEALHNHYTQKSLSLSPG |
| | 21 | CAGTCCACAATCGAGGAGCAGGCCAAGACCTTCCTGGACA |
| | | AGTTTAACCACGAGGCCGAGGATCTGTTCTATCAGAGCTCC |
| | | CTGGCCAGCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGTCTGAGGAT |
| | | AAGAGCAAGCGGCTGAATACAATCCTGAACACCATGTCCA |
| | | CAATCTATTCTACCGGCAAGGTGTGCAATCCCGACAACCCT |
| | | CAGGAGTGTCTGCTGCTGGAGCCTGGCCTGAATGAGATCA |
| | | TGGCCAACAGCCTGGATTACAATGAGAGGCTGTGGGCATG |
| | | GGAGAGCTGGCGCTCCGAAGTGGGCAAGCAGCTGCGGCCA |
| | | CTGTACGAGGAGTATGTGGTGCTGAAGAACGAGATGGCCA |
| | | GAGCCAATCACTACGAGGACTATGGCGATTACTGGAGGGG |
| | | CGACTACGAGGTGAACGGCGTGGACGGCTACGATTATTCC |
| | | AGAGGCCAGCTGATCGAGGATGTGGAGCACACATTCGAGG |
| | | AGATCAAGCCACTGTATGAGCACCTGCACGCCTACGTGAG |
| | | GGCCAAGCTGATGAACGCCTATCCTTCTTACATCAGCCCAA |
| | | TCGGATGCCTGCCCGCCCACCTGCTGGGCGACATGTGGGG |
| | | CCGCTTCTGGACAAACCTGTACAGCCTGACCGTGCCTTTTG |
| | | GCCAGAAGCCAAATATCGACGTGACCGATGCCATGGTGGA |
| | | CCAGGCCTGGGATGCCCAGCGGATCTTCAAGGAGGCCGAG |
| | | AAGTTCTTCGTGAGCGTGGGCCTGCCAAACATGACACAGG |
| | | GCTTTTGGGAGAACAGCATGCTGACCGACCCCGGCAACGT |
| | | GCAGAAGGCCGTGTGCCACCCTACAGCCTGGGACCTGGGC |
| | | AAGGGCGATTTCAGAATCCTGATGTGCACCAAGGTGACAA |
| | | TGGACGATTTTCTGACCGCCCACCACGAGATGGGCCACATC |
| | | CAGTATGATATGGCATACGCAGCACAGCCCTTCCTGCTGAG |
| | | GAATGGAGCCAACGAGGGCTTTCACGAGGCCGTGGGCGAG |
| | | ATCATGTCTCTGAGCGCCGCCACACCAAAGCACCTGAAGT |
| | | CCATCGGCCTGCTGTCTCCCGACTTCCAGGAGGATAACGAG |
| | | ACCGAGATCAATTTTCTGCTGAAGCAGGCCCTGACCATCGT |
| | | GGGCACACTGCCCTTCACCTATATGCTGGAGAAGTGGAGA |
| | | TGGATGGTGTTTAAGGGCGAGATCCCTAAGGACCAGTGGA |
| | | TGAAGAAGTGGTGGGAGATGAAGAGGGAGATCGTGGGAG |
| | | TGGTGGAGCCCGTGCCTCACGACGAGACCTACTGTGATCCC |
| | | GCCTCCCTGTTCCACGTGAGCAACGACTATTCCTTTATCAG |
| | | GTACTATACCCGCACACTGTACCAGTTCCAGTTTCAGGAGG |
| | | CCCTGTGCCAGGCAGCAAAGCACGAGGGCCCACTGCACAA |
| | | GTGTGATATCTCCAACTCTACAGAGGCCGGACAGAAGCTG |
| | | TTCAATATGCTGCGCCTGGGCAAGAGCGAGCCATGGACCC |
| | | TGGCCCTGGAGAACGTGGTGGGAGCCAAGAATATGAACGT |
| | | GCGGCCCCTGCTGAATTATTTCGAGCCTCTGTTTACATGGC |
| | | TGAAGGATCAGAATAAGAACAGCTTTGTGGGCTGGTCTAC |
| | | CGACTGGAGCCCCTACGCCGATCAGTCCATCAAGGTGAGA |
| | | ATCTCCCTGAAGTCTGCCCTGGGCGACAAGGCCTATGAGTG |
| | | GAATGATAACGAGATGTACCTGTTCAGATCCTCTGTGGCCT |
| | | ATGCCATGAGGCAGTACTTCCTGAAGGTGAAGAACCAGAT |
| | | GATCCTGTTTGGCGAGGAGGACGTGCGGGTGGCCAATCTG |
| | | AAGCCTAGAATCAGCTTTAACTTCTTTGTGACAGCCCCAAA |
| | | GAACGTGAGCGATATCATCCCCAGAACCGAGGTGGAGAAG |
| | | GCCATCCGGATGAGCCGGTCCAGAATCAACGACGCCTTCC |
| | | GCCTGAATGATAACTCCCTGGAGTTTCTGGGCATCCAGCCT |
| | | ACCCTGGGCCCACCTAATCAGCCACCCGTGAGCGAGCCAA |
| | | AGAGCTCCGACAAGACCCACACATGCCCTCCATGTCCGGC |
| | | GCCAGAGGCCGCCGGAGGACCTAGCGTGTTCCTGTTTCCCC |
| | | CTAAGCCAAAGGATACACTGATGATCAGCCGGACACCAGA |
| | | GGTGACCTGCGTGGTGGTGTCTGTGAGCCACGAGGACCCC |
| | | GAGGTGAAGTTCAACTGGTATGTGGATGGCGTGGAGGTGC |
| | | ACAATGCCAAGACCAAGCCTAGAGAGGAGCAGTATAACAG |
| | | CACATACAGGGTGGTGTCCGTGCTGACCGTGCTGCACCAG |
| | | GACTGGCTGAACGGCAAGGAGTACAAGTGCAAGGTGTCTA |
| | | ATAAGGCCCTGCCTGCCCCAATCGAGAAGACCATCAGCAA |
| | | GGCAAAGGGACAGCCTCGCGAACCACAGGTCTACGTGCTG |
| | | CCCCCTAGCCGCGACGAACTGACTAAAAATCAGGTCTCTCT |
| | | GCTGTGTCTGGTCAAAGGATTCTACCCTTCCGACATCGCCG |
| | | TGGAGTGGGAAAGTAACGGCCAGCCCGAGAACAATTACCT |
| | | GACCTGGCCCCCTGTGCTGGACTCTGATGGGAGTTTCTTTC |
| | | TGTATTCAAAGCTGACAGTCGATAAAAGCCGGTGGCAGCA |
| | | GGGCAATGTGTTCAGCTGCTCCGTCATGCACGAAGCACTGC |
| | | ACAACCATTACACTCAGAAGTCCCTGTCCCTGTCACCTGGC |
| 26012 | 22 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSEPKSSDKTHTCPPCPAPEAAGGPSVFLF |
| | | PPKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYVLPPSRDELTKNQVSLLCL |
| | | VKGFYPSDIAVEWESNGQPENNYLTWPPVLDSDGSFFLYSKL |
| | | TVDKSRWQQGNVFSCSVMHEALHNHYTQKSLSLSPG |
| | 23 | CAGTCCACAATCGAGGAGCAGGCCAAGACCTTCCTGGACA |
| | | AGTTTAACCACGAGGCCGAGGATCTGTTCTATCAGAGCTCC |
| | | CTGGCCTCTTGGAACTACAATACCAACATCACAGAGGAGA |
| | | ATGTGCAGAACATGAACAATGCCGGGGACAAGTGGAGCGC |
| | | CTTTCTGAAGGAGCAGTCCACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGTCTGAGGAT |
| | | AAGAGCAAGAGGCTGAATACAATCCTGAACACCATGTCCA |
| | | CAATCTATTCTACCGGCAAGGTGTGCAATCCAGACAACCCC |
| | | CAGGAGTGTCTGCTGCTGGAGCCTGGCCTGAATGAGATCA |
| | | TGGCCAACAGCCTGGATTACAATGAGAGGCTGTGGGCATG |
| | | GGAGTCTTGGCGCAGCGAAGTGGGCAAGCAGCTGCGGCCA |
| | | CTGTACGAGGAGTATGTGGTGCTGAAGAACGAGATGGCCA |
| | | GAGCCAATCACTACGAGGACTATGGCGATTACTGGAGGGG |
| | | CGACTACGAGGTGAACGGCGTGGACGGCTACGATTATAGC |
| | | AGAGGCCAGCTGATCGAGGATGTGGAGCACACATTCGAGG |
| | | AGATCAAGCCCCTGTATGAGCACCTGCACGCCTACGTGAG |
| | | GGCCAAGCTGATGAACGCCTATCCTTCCTACATCTCTCCAA |
| | | TCGGATGCCTGCCCGCCCACCTGCTGGGCGACATGTGGGG |
| | | CCGCTTCTGGACAAACCTGTACTCCCTGACCGTGCCTTTTG |
| | | GCCAGAAGCCAAATATCGACGTGACCGATGCCATGGTGGA |
| | | CCAGGCCTGGGATGCCCAGCGGATCTTCAAGGAGGCCGAG |
| | | AAGTTCTTCGTGAGCGTGGGCCTGCCAAACATGACACAGG |
| | | GCTTTTGGGAGAACAGCATGCTGACCGACCCTGGCAACGT |
| | | GCAGAAGGCCGTGTGCCACCCAACAGCCTGGGACCTGGGC |
| | | AAGGGCGATTTCAGAATCCTGATGTGCACCAAGGTGACAA |
| | | TGGACGATTTTCTGACCGCCCACAATGAGATGGGCAACAT |
| | | CCAGTATGATATGGCATACGCAGCACAGCCCTTCCTGCTGA |
| | | GGAATGGAGCCAACGAGGGCTTTCACGAGGCCGTGGGCGA |
| | | GATCATGTCTCTGAGCGCCGCCACACCCAAGCACCTGAAG |
| | | TCCATCGGCCTGCTGTCTCCTGACTTCCAGGAGGATAACGA |
| | | GACCGAGATCAATTTTCTGCTGAAGCAGGCCCTGACCATCG |
| | | TGGGCACACTGCCCTTCACCTATATGCTGGAGAAGTGGAG |
| | | ATGGATGGTGTTTAAGGGCGAGATCCCTAAGGACCAGTGG |
| | | ATGAAGAAGTGGTGGGAGATGAAGAGGGAGATCGTGGGA |
| | | GTGGTGGAGCCCGTGCCTCACGACGAGACCTACTGTGATC |
| | | CCGCCAGCCTGTTCCACGTGAGCAACGACTATTCCTTTATC |
| | | AGGTACTATACCCGCACACTGTACCAGTTCCAGTTTCAGGA |
| | | GGCCCTGTGCCAGGCAGCAAAGCACGAGGGCCCACTGCAC |
| | | AAGTGTGATATCTCCAACTCTACAGAGGCCGGACAGAAGC |
| | | TGTTCAATATGCTGCGCCTGGGCAAGTCCGAGCCATGGACC |
| | | CTGGCCCTGGAGAACGTGGTGGGAGCCAAGAATATGAACG |
| | | TGCGGCCCCTGCTGAATTATTTCGAGCCTCTGTTTACATGG |
| | | CTGAAGGATCAGAATAAGAACAGCTTTGTGGGCTGGTCTA |
| | | CCGACTGGAGCCCCTACGCCGATCAGTCCATCAAGGTGCG |
| | | CATCAGCCTGAAGTCCGCCCTGGGCGACAAGGCCTATGAG |
| | | TGGAATGATAACGAGATGTACCTGTTCAGATCCTCTGTGGC |
| | | CTATGCCATGAGGCAGTACTTCCTGAAGGTGAAGAACCAG |
| | | ATGATCCTGTTTGGCGAGGAGGACGTGCGGGTGGCCAATC |
| | | TGAAGCCTAGAATCTCTTTTAACTTCTTTGTGACAGCCCCA |
| | | AAGAATGTGAGCGATATCATCCCCAGAACCGAGGTGGAGA |
| | | AGGCCATCCGGATGAGCCGGTCCAGAATCAACGACGCCTT |
| | | CCGGCTGAATGATAACTCTCTGGAGTTTCTGGGCATCCAGC |
| | | CAACCCTGGGCCCACCTAATCAGCCACCCGTGAGCGAGCC |
| | | CAAGAGCTCCGACAAGACCCACACATGCCCTCCATGTCCG |
| | | GCGCCAGAGGCCGCCGGAGGACCAAGCGTGTTCCTGTTTC |
| | | CACCCAAGCCCAAGGACACCCTGATGATCTCCCGGACCCC |
| | | AGAGGTGACATGCGTGGTGGTGAGCGTGTCCCACGAGGAC |
| | | CCCGAGGTGAAGTTTAACTGGTACGTGGATGGCGTGGAGG |
| | | TGCACAATGCCAAGACAAAGCCCCGGGAGGAGCAGTACAA |
| | | TTCTACCTATAGAGTGGTGAGCGTGCTGACAGTGCTGCACC |
| | | AGGATTGGCTGAACGGCAAGGAGTATAAGTGTAAGGTGAG |
| | | CAATAAGGCCCTGCCAGCCCCCATCGAGAAGACCATCTCC |
| | | AAGGCCAAGGGCCAGCCTCGCGAACCACAGGTCTACGTGC |
| | | TGCCCCCTAGCCGCGACGAACTGACTAAAAATCAGGTCTCT |
| | | CTGCTGTGTCTGGTCAAAGGATTCTACCCTTCCGACATCGC |
| | | CGTGGAGTGGGAAAGTAACGGCCAGCCCGAGAACAATTAC |
| | | CTGACCTGGCCCCCTGTGCTGGACTCTGATGGGAGTTTCTT |
| | | TCTGTATTCAAAGCTGACAGTCGATAAAAGCCGGTGGCAG |
| | | CAGGGCAATGTGTTCAGCTGCTCCGTCATGCACGAAGCACT |
| | | GCACAACCATTACACTCAGAAGTCCCTGTCCCTGTCACCTG |
| | | GC |
| 26009 | 24 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHHEMGHIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSVPPPPPCCHPRLSLHRPALEDLLLGSEA |
| | | NLTCTLTGLRDASGVTFTWTPSSGKSAVQGPPERDLCGCYSV |
| | | SSVLPGSAEPWNHGKTFTCTAAYPESKTPLTATLSKSGNTFRP |
| | | EVHLLPPPSEELALNELVTLTCLARGFSPKDVLVRWLOGSQEL |
| | | PREKYLTWASRQEPSQGTTTFAVTSILRVAAEDWKKGDTFSC |
| | | MVGHEALPLAFTQKTIDRLAGKPTHVNVSVVMAEVDGTCY |
| | 25 | CAGTCTACAATCGAGGAGCAGGCCAAGACCTTCCTGGACA |
| | | AGTTTAACCACGAGGCCGAGGATCTGTTCTATCAGAGCTCC |
| | | CTGGCCTCCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCCC |
| | | TGCAGGAGATCCAGAACCTGACCGTGAAGCTGCAGCTGCA |
| | | GGCCCTGCAGCAGAATGGCTCTAGCGTGCTGAGCGAGGAT |
| | | AAGTCCAAGAGGCTGAATACAATCCTGAACACCATGTCTA |
| | | CAATCTATAGCACCGGCAAGGTGTGCAATCCAGACAACCC |
| | | CCAGGAGTGTCTGCTGCTGGAGCCAGGCCTGAATGAGATC |
| | | ATGGCCAACAGCCTGGATTACAATGAGAGGCTGTGGGCAT |
| | | GGGAGAGCTGGCGCTCCGAAGTGGGCAAGCAGCTGAGACC |
| | | CCTGTACGAGGAGTATGTGGTGCTGAAGAACGAGATGGCC |
| | | AGAGCCAATCACTACGAGGACTATGGCGATTACTGGAGGG |
| | | GCGACTACGAGGTGAACGGCGTGGACGGCTACGATTATAG |
| | | CAGAGGCCAGCTGATCGAGGATGTGGAGCACACATTCGAG |
| | | GAGATCAAGCCCCTGTATGAGCACCTGCACGCCTACGTGA |
| | | GGGCCAAGCTGATGAACGCCTATCCTTCTTACATCAGCCCA |
| | | ATCGGATGCCTGCCCGCCCACCTGCTGGGCGACATGTGGG |
| | | GCCGCTTCTGGACAAACCTGTACTCCCTGACCGTGCCTTTT |
| | | GGCCAGAAGCCAAATATCGACGTGACCGATGCCATGGTGG |
| | | ACCAGGCCTGGGATGCCCAGCGGATCTTCAAGGAGGCCGA |
| | | GAAGTTCTTCGTGAGCGTGGGCCTGCCCAACATGACACAG |
| | | GGCTTTTGGGAGAATAGCATGCTGACCGACCCTGGCAACG |
| | | TGCAGAAGGCCGTGTGCCACCCAACAGCCTGGGACCTGGG |
| | | CAAGGGCGATTTCAGAATCCTGATGTGCACCAAGGTGACA |
| | | ATGGACGATTTTCTGACCGCCCACCACGAGATGGGCCACA |
| | | TCCAGTATGATATGGCCTACGCCGCCCAGCCTTTCCTGCTG |
| | | AGGAATGGAGCCAACGAGGGCTTTCACGAGGCCGTGGGCG |
| | | AGATCATGAGCCTGTCCGCCGCCACACCCAAGCACCTGAA |
| | | GTCTATCGGCCTGCTGAGCCCTGACTTCCAGGAGGATAACG |
| | | AGACCGAGATCAATTTTCTGCTGAAGCAGGCCCTGACCATC |
| | | GTGGGCACACTGCCTTTCACCTATATGCTGGAGAAGTGGA |
| | | GATGGATGGTGTTTAAGGGCGAGATCCCAAAGGACCAGTG |
| | | GATGAAGAAGTGGTGGGAGATGAAGAGGGAGATCGTGGG |
| | | AGTGGTGGAGCCCGTGCCTCACGACGAGACCTACTGTGAT |
| | | CCAGCCAGCCTGTTCCACGTGTCCAACGACTATTCTTTTAT |
| | | CAGGTACTATACCCGCACACTGTACCAGTTCCAGTTTCAGG |
| | | AGGCCCTGTGCCAGGCAGCAAAGCACGAGGGACCACTGCA |
| | | CAAGTGTGATATCTCTAACAGCACAGAGGCCGGACAGAAG |
| | | CTGTTCAATATGCTGCGCCTGGGCAAGTCCGAGCCTTGGAC |
| | | CCTGGCCCTGGAGAACGTGGTGGGAGCCAAGAATATGAAC |
| | | GTGCGGCCCCTGCTGAATTATTTCGAGCCTCTGTTTACATG |
| | | GCTGAAGGATCAGAATAAGAACTCCTTTGTGGGCTGGAGC |
| | | ACCGACTGGTCCCCTTACGCCGATCAGTCTATCAAGGTGCG |
| | | CATCTCCCTGAAGTCTGCCCTGGGCGACAAGGCCTATGAGT |
| | | GGAATGATAACGAGATGTACCTGTTCAGATCCTCTGTGGCC |
| | | TATGCCATGAGGCAGTACTTCCTGAAGGTGAAGAACCAGA |
| | | TGATCCTGTTTGGCGAGGAGGACGTGCGGGTGGCCAATCT |
| | | GAAGCCTAGAATCAGCTTTAACTTCTTTGTGACAGCCCCAA |
| | | AGAACGTGAGCGATATCATCCCCAGAACCGAGGTGGAGAA |
| | | GGCCATCCGGATGTCCCGGTCTAGAATCAACGACGCCTTCC |
| | | GGCTGAATGATAACAGCCTGGAGTTTCTGGGCATCCAGCC |
| | | AACCCTGGGCCCACCTAATCAGCCACCCGTGTCCGTGCCTC |
| | | CACCACCTCCATGCTGTCACCCTAGGCTGTCCCTGCACAGA |
| | | CCAGCCCTGGAGGACCTGCTGCTGGGCTCTGAGGCCAATCT |
| | | GACATGTACCCTGACCGGCCTGAGGGATGCCAGCGGAGTG |
| | | ACCTTCACATGGACCCCATCTAGCGGCAAGTCCGCCGTGCA |
| | | GGGACCTCCAGAGAGGGATCTGTGCGGATGTTACTCCGTG |
| | | TCCTCTGTGCTGCCAGGCTCTGCCGAGCCTTGGAACCACGG |
| | | CAAGACATTCACCTGCACAGCCGCCTATCCAGAGTCCAAG |
| | | ACACCCCTGACCGCCACACTGTCTAAGAGCGGCAACACCT |
| | | TTCGGCCGGAGGTGCACCTGCTGCCACCTCCATCTGAGGAG |
| | | CTGGCCCTGAATGAGCTGGTGACCCTGACATGTCTGGCCCG |
| | | GGGCTTCTCTCCTAAGGATGTGCTGGTGAGATGGCTGCAGG |
| | | GCAGCCAGGAGCTGCCAAGGGAGAAGTACCTGACCTGGGC |
| | | CTCTCGCCAGGAGCCAAGCCAGGGAACCACAACCTTTGCC |
| | | GTGACCTCTATCCTGCGGGTGGCCGCCGAGGACTGGAAGA |
| | | AGGGCGATACCTTCAGCTGCATGGTGGGACACGAGGCCCT |
| | | GCCTCTGGCCTTTACCCAGAAGACAATCGACCGGTTAGCCG |
| | | GGAAGCCAACACACGTGAATGTGAGCGTGGTCATGGCCGA |
| | | GGTGGATGGCACCTGTTAT |
| 26013 | 26 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSVPPPPPCCHPRLSLHRPALEDLLLGSEA |
| | | NLTCTLTGLRDASGVTFTWTPSSGKSAVQGPPERDLCGCYSV |
| | | SSVLPGSAEPWNHGKTFTCTAAYPESKTPLTATLSKSGNTFRP |
| | | EVHLLPPPSEELALNELVTLTCLARGFSPKDVLVRWLQGSQEL |
| | | PREKYLTWASRQEPSQGTTTFAVTSILRVAAEDWKKGDTFSC |
| | | MVGHEALPLAFTQKTIDRLAGKPTHVNVSVVMAEVDGTCY |
| | 27 | CAGTCTACAATCGAGGAGCAGGCCAAGACCTTCCTGGACA |
| | | AGTTTAACCACGAGGCCGAGGATCTGTTCTATCAGAGCTCC |
| | | CTGGCCTCCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCCC |
| | | TGCAGGAGATCCAGAACCTGACCGTGAAGCTGCAGCTGCA |
| | | GGCCCTGCAGCAGAATGGCTCTAGCGTGCTGAGCGAGGAT |
| | | AAGTCCAAGAGGCTGAATACAATCCTGAACACCATGTCTA |
| | | CAATCTATAGCACCGGCAAGGTGTGCAATCCAGACAACCC |
| | | CCAGGAGTGTCTGCTGCTGGAGCCAGGCCTGAATGAGATC |
| | | ATGGCCAACAGCCTGGATTACAATGAGAGGCTGTGGGCAT |
| | | GGGAGAGCTGGCGCTCCGAAGTGGGCAAGCAGCTGAGACC |
| | | CCTGTACGAGGAGTATGTGGTGCTGAAGAACGAGATGGCC |
| | | AGAGCCAATCACTACGAGGACTATGGCGATTACTGGAGGG |
| | | GCGACTACGAGGTGAACGGCGTGGACGGCTACGATTATAG |
| | | CAGAGGCCAGCTGATCGAGGATGTGGAGCACACATTCGAG |
| | | GAGATCAAGCCCCTGTATGAGCACCTGCACGCCTACGTGA |
| | | GGGCCAAGCTGATGAACGCCTATCCTTCTTACATCAGCCCA |
| | | ATCGGATGCCTGCCCGCCCACCTGCTGGGCGACATGTGGG |
| | | GCCGCTTCTGGACAAACCTGTACTCCCTGACCGTGCCTTTT |
| | | GGCCAGAAGCCAAATATCGACGTGACCGATGCCATGGTGG |
| | | ACCAGGCCTGGGATGCCCAGCGGATCTTCAAGGAGGCCGA |
| | | GAAGTTCTTCGTGAGCGTGGGCCTGCCCAACATGACACAG |
| | | GGCTTTTGGGAGAATAGCATGCTGACCGACCCTGGCAACG |
| | | TGCAGAAGGCCGTGTGCCACCCAACAGCCTGGGACCTGGG |
| | | CAAGGGCGATTTCAGAATCCTGATGTGCACCAAGGTGACA |
| | | ATGGACGATTTTCTGACCGCCCACAACGAGATGGGCAACA |
| | | TCCAGTATGATATGGCCTACGCCGCCCAGCCTTTCCTGCTG |
| | | AGGAATGGAGCCAACGAGGGCTTTCACGAGGCCGTGGGCG |
| | | AGATCATGAGCCTGTCCGCCGCCACACCCAAGCACCTGAA |
| | | GTCTATCGGCCTGCTGAGCCCTGACTTCCAGGAGGATAACG |
| | | AGACCGAGATCAATTTTCTGCTGAAGCAGGCCCTGACCATC |
| | | GTGGGCACACTGCCTTTCACCTATATGCTGGAGAAGTGGA |
| | | GATGGATGGTGTTTAAGGGCGAGATCCCAAAGGACCAGTG |
| | | GATGAAGAAGTGGTGGGAGATGAAGAGGGAGATCGTGGG |
| | | AGTGGTGGAGCCCGTGCCTCACGACGAGACCTACTGTGAT |
| | | CCAGCCAGCCTGTTCCACGTGTCCAACGACTATTCTTTTAT |
| | | CAGGTACTATACCCGCACACTGTACCAGTTCCAGTTTCAGG |
| | | AGGCCCTGTGCCAGGCAGCAAAGCACGAGGGACCACTGCA |
| | | CAAGTGTGATATCTCTAACAGCACAGAGGCCGGACAGAAG |
| | | CTGTTCAATATGCTGCGCCTGGGCAAGTCCGAGCCTTGGAC |
| | | CCTGGCCCTGGAGAACGTGGTGGGAGCCAAGAATATGAAC |
| | | GTGCGGCCCCTGCTGAATTATTTCGAGCCTCTGTTTACATG |
| | | GCTGAAGGATCAGAATAAGAACTCCTTTGTGGGCTGGAGC |
| | | ACCGACTGGTCCCCTTACGCCGATCAGTCTATCAAGGTGCG |
| | | CATCTCCCTGAAGTCTGCCCTGGGCGACAAGGCCTATGAGT |
| | | GGAATGATAACGAGATGTACCTGTTCAGATCCTCTGTGGCC |
| | | TATGCCATGAGGCAGTACTTCCTGAAGGTGAAGAACCAGA |
| | | TGATCCTGTTTGGCGAGGAGGACGTGCGGGTGGCCAATCT |
| | | GAAGCCTAGAATCAGCTTTAACTTCTTTGTGACAGCCCCAA |
| | | AGAACGTGAGCGATATCATCCCCAGAACCGAGGTGGAGAA |
| | | GGCCATCCGGATGTCCCGGTCTAGAATCAACGACGCCTTCC |
| | | GGCTGAATGATAACAGCCTGGAGTTTCTGGGCATCCAGCC |
| | | AACCCTGGGCCCACCTAATCAGCCACCCGTGTCCGTGCCTC |
| | | CACCACCTCCATGCTGTCACCCTAGGCTGTCCCTGCACAGA |
| | | CCAGCCCTGGAGGACCTGCTGCTGGGCTCTGAGGCCAATCT |
| | | GACATGTACCCTGACCGGCCTGAGGGATGCCAGCGGAGTG |
| | | ACCTTCACATGGACCCCATCTAGCGGCAAGTCCGCCGTGCA |
| | | GGGACCTCCAGAGAGGGATCTGTGCGGATGTTACTCCGTG |
| | | TCCTCTGTGCTGCCAGGCTCTGCCGAGCCTTGGAACCACGG |
| | | CAAGACATTCACCTGCACAGCCGCCTATCCAGAGTCCAAG |
| | | ACACCCCTGACCGCCACACTGTCTAAGAGCGGCAACACCT |
| | | TTCGGCCGGAGGTGCACCTGCTGCCACCTCCATCTGAGGAG |
| | | CTGGCCCTGAATGAGCTGGTGACCCTGACATGTCTGGCCCG |
| | | GGGCTTCTCTCCTAAGGATGTGCTGGTGAGATGGCTGCAGG |
| | | GCAGCCAGGAGCTGCCAAGGGAGAAGTACCTGACCTGGGC |
| | | CTCTCGCCAGGAGCCAAGCCAGGGAACCACAACCTTTGCC |
| | | GTGACCTCTATCCTGCGGGTGGCCGCCGAGGACTGGAAGA |
| | | AGGGCGATACCTTCAGCTGCATGGTGGGACACGAGGCCCT |
| | | GCCTCTGGCCTTTACCCAGAAGACAATCGACCGGTTAGCCG |
| | | GGAAGCCAACACACGTGAATGTGAGCGTGGTCATGGCCGA |
| | | GGTGGATGGCACCTGTTAT |
| 26010 | 28 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHHEMGHIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSVIAELPPKVSVFVPPRDGFFGNPRKSKLI |
| | | CQATGFSPRQIQVSWLREGKQVGSGVTTDQVQAEAKESGPTT |
| | | YKVTSTLTIKESDWLSQSMFTCRVDHRGLTFQQNASSMCVPD |
| | | QDTAIRVFAIPPSFASIFLTKSTKLTCLVTDLTTYDSVTISWTRQ |
| | | NGEAVKTHTNISESHPNATFSAVGEASICEDDWNSGERFTCTV |
| | | THTDLPSPLKQTISRPKGVALHRPDVYLLPPAREQLNLRESATI |
| | | TCLVTGFSPADVFVQWMQRGQPLSPEKYVTSAPMPEPQAPGR |
| | | YFAHSILTVSEEEWNTGETYTCVVAHEALPNRVTERTVDKST |
| | | GKPTLYNVSLVMSDTAGTCY |
| | 29 | CAGTCTACAATCGAGGAGCAGGCCAAGACCTTCCTGGACA |
| | | AGTTTAACCACGAGGCCGAGGATCTGTTCTATCAGAGCTCC |
| | | CTGGCCAGCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGAGCGAGGAT |
| | | AAGTCCAAGAGGCTGAATACAATCCTGAACACCATGTCTA |
| | | CAATCTATAGCACCGGCAAGGTGTGCAATCCAGACAACCC |
| | | CCAGGAGTGTCTGCTGCTGGAGCCTGGCCTGAATGAGATC |
| | | ATGGCCAACAGCCTGGATTACAATGAGAGGCTGTGGGCAT |
| | | GGGAGAGCTGGCGCTCCGAAGTGGGCAAGCAGCTGCGGCC |
| | | ACTGTACGAGGAGTATGTGGTGCTGAAGAACGAGATGGCC |
| | | AGAGCCAATCACTACGAGGACTATGGCGATTACTGGAGGG |
| | | GCGACTACGAGGTGAACGGCGTGGACGGCTACGATTATAG |
| | | CAGAGGCCAGCTGATCGAGGATGTGGAGCACACATTCGAG |
| | | GAGATCAAGCCCCTGTATGAGCACCTGCACGCCTACGTGA |
| | | GGGCCAAGCTGATGAACGCCTATCCTTCTTACATCAGCCCA |
| | | ATCGGATGCCTGCCCGCCCACCTGCTGGGCGACATGTGGG |
| | | GCCGCTTCTGGACAAACCTGTACTCCCTGACCGTGCCTTTT |
| | | GGCCAGAAGCCAAATATCGACGTGACCGATGCCATGGTGG |
| | | ACCAGGCCTGGGATGCCCAGCGGATCTTCAAGGAGGCCGA |
| | | GAAGTTCTTCGTGAGCGTGGGCCTGCCAAACATGACACAG |
| | | GGCTTTTGGGAGAACAGCATGCTGACCGACCCTGGCAACG |
| | | TGCAGAAGGCCGTGTGCCACCCAACAGCCTGGGACCTGGG |
| | | CAAGGGCGATTTCAGAATCCTGATGTGCACCAAGGTGACA |
| | | ATGGACGATTTTCTGACCGCCCACCACGAGATGGGCCACA |
| | | TCCAGTATGATATGGCATACGCAGCACAGCCCTTCCTGCTG |
| | | AGGAATGGAGCCAACGAGGGCTTTCACGAGGCCGTGGGCG |
| | | AGATCATGAGCCTGTCCGCCGCCACACCCAAGCACCTGAA |
| | | GTCTATCGGCCTGCTGAGCCCTGACTTCCAGGAGGATAACG |
| | | AGACCGAGATCAATTTTCTGCTGAAGCAGGCCCTGACCATC |
| | | GTGGGCACACTGCCTTTCACCTATATGCTGGAGAAGTGGA |
| | | GATGGATGGTGTTTAAGGGCGAGATCCCAAAGGACCAGTG |
| | | GATGAAGAAGTGGTGGGAGATGAAGAGGGAGATCGTGGG |
| | | AGTGGTGGAGCCCGTGCCTCACGACGAGACCTACTGTGAT |
| | | CCCGCCTCCCTGTTCCACGTGTCCAACGACTATTCTTTTATC |
| | | AGGTACTATACCCGCACACTGTACCAGTTCCAGTTTCAGGA |
| | | GGCCCTGTGCCAGGCAGCAAAGCACGAGGGCCCACTGCAC |
| | | AAGTGTGATATCTCTAACAGCACAGAGGCCGGACAGAAGC |
| | | TGTTCAATATGCTGCGCCTGGGCAAGTCTGAGCCCTGGACC |
| | | CTGGCCCTGGAGAACGTGGTGGGAGCCAAGAATATGAACG |
| | | TGCGGCCCCTGCTGAATTATTTCGAGCCTCTGTTTACATGG |
| | | CTGAAGGATCAGAATAAGAACAGCTTTGTGGGATGGAGCA |
| | | CCGACTGGTCCCCATACGCCGATCAGTCCATCAAGGTGCGC |
| | | ATCTCCCTGAAGTCTGCCCTGGGCGACAAGGCCTATGAGTG |
| | | GAATGATAACGAGATGTACCTGTTCAGATCCTCTGTGGCCT |
| | | ATGCCATGAGGCAGTACTTCCTGAAGGTGAAGAACCAGAT |
| | | GATCCTGTTTGGCGAGGAGGACGTGCGGGTGGCCAATCTG |
| | | AAGCCTAGAATCTCTTTTAACTTCTTTGTGACAGCCCCAAA |
| | | GAATGTGAGCGATATCATCCCCAGAACCGAGGTGGAGAAG |
| | | GCCATCCGGATGTCCCGGTCTAGAATCAACGACGCCTTCCG |
| | | GCTGAATGATAACTCCCTGGAGTTTCTGGGCATCCAGCCCA |
| | | CCCTGGGCCCACCTAATCAGCCACCCGTGAGCGTGATCGC |
| | | AGAGCTGCCTCCAAAGGTGTCCGTGTTCGTGCCTCCTAGGG |
| | | ACGGCTTCTTTGGCAATCCACGCAAGAGCAAGCTGATCTGT |
| | | CAGGCCACCGGCTTTTCTCCCAGACAGATCCAGGTGAGCTG |
| | | GCTGAGAGAGGGCAAGCAAGTGGGCTCCGGAGTGACCACA |
| | | GATCAGGTGCAGGCAGAGGCAAAGGAGTCTGGACCAACCA |
| | | CATACAAGGTGACCTCCACACTGACCATCAAGGAGTCTGA |
| | | CTGGCTGAGCCAGTCCATGTTCACCTGCCGGGTGGATCACA |
| | | GAGGCCTGACATTTCAGCAGAACGCCAGCTCCATGTGCGT |
| | | GCCTGACCAGGATACCGCCATCAGAGTGTTCGCGATCCCTC |
| | | CATCCTTCGCCTCTATCTTTCTGACAAAGTCCACCAAGCTG |
| | | ACATGCCTGGTGACCGACCTGACCACATATGATTCCGTGAC |
| | | CATCTCTTGGACAAGGCAGAACGGCGAGGCCGTGAAGACA |
| | | CACACCAACATCTCTGAGAGCCACCCCAATGCCACCTTCAG |
| | | CGCCGTGGGAGAGGCATCCATCTGCGAGGACGATTGGAAT |
| | | AGCGGCGAGCGGTTTACATGTACCGTGACACACACCGACC |
| | | TGCCTAGCCCACTGAAGCAGACCATCTCCAGGCCTAAGGG |
| | | AGTGGCCCTGCACCGGCCGGACGTGTACCTGCTGCCCCCTG |
| | | CCAGGGAGCAGCTGAACCTGAGAGAGAGCGCCACAATCAC |
| | | CTGTCTGGTGACCGGCTTCTCCCCTGCCGACGTGTTTGTGC |
| | | AGTGGATGCAGAGGGGACAGCCCCTGTCTCCTGAGAAGTA |
| | | CGTGACAAGCGCCCCCATGCCTGAGCCACAGGCCCCAGGC |
| | | CGGTATTTCGCCCACTCTATCCTGACCGTGAGCGAGGAGGA |
| | | GTGGAACACAGGCGAGACATATACCTGCGTGGTGGCACAC |
| | | GAGGCCCTGCCTAATAGGGTGACCGAGCGCACAGTGGACA |
| | | AGAGCACCGGTAAGCCAACACTGTACAATGTGAGCCTGGT |
| | | CATGTCCGATACCGCCGGGACCTGTTAT |
| 26014 | 30 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSVIAELPPKVSVFVPPRDGFFGNPRKSKLI |
| | | CQATGFSPRQIQVSWLREGKQVGSGVTTDQVQAEAKESGPTT |
| | | YKVTSTLTIKESDWLSQSMFTCRVDHRGLTFQQNASSMCVPD |
| | | QDTAIRVFAIPPSFASIFLTKSTKLTCLVTDLTTYDSVTISWTRQ |
| | | NGEAVKTHTNISESHPNATFSAVGEASICEDDWNSGERFTCTV |
| | | THTDLPSPLKQTISRPKGVALHRPDVYLLPPAREQLNLRESATI |
| | | TCLVTGFSPADVFVQWMQRGQPLSPEKYVTSAPMPEPQAPGR |
| | | YFAHSILTVSEEEWNTGETYTCVVAHEALPNRVTERTVDKST |
| | | GKPTLYNVSLVMSDTAGTCY |
| | 31 | CAGTCTACAATCGAGGAGCAGGCCAAGACCTTCCTGGACA |
| | | AGTTTAACCACGAGGCCGAGGATCTGTTCTATCAGAGCTCC |
| | | CTGGCCAGCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGAGCGAGGAT |
| | | AAGTCCAAGAGGCTGAATACAATCCTGAACACCATGTCTA |
| | | CAATCTATAGCACCGGCAAGGTGTGCAATCCAGACAACCC |
| | | CCAGGAGTGTCTGCTGCTGGAGCCTGGCCTGAATGAGATC |
| | | ATGGCCAACAGCCTGGATTACAATGAGAGGCTGTGGGCAT |
| | | GGGAGAGCTGGCGCTCCGAAGTGGGCAAGCAGCTGCGGCC |
| | | ACTGTACGAGGAGTATGTGGTGCTGAAGAACGAGATGGCC |
| | | AGAGCCAATCACTACGAGGACTATGGCGATTACTGGAGGG |
| | | GCGACTACGAGGTGAACGGCGTGGACGGCTACGATTATAG |
| | | CAGAGGCCAGCTGATCGAGGATGTGGAGCACACATTCGAG |
| | | GAGATCAAGCCCCTGTATGAGCACCTGCACGCCTACGTGA |
| | | GGGCCAAGCTGATGAACGCCTATCCTTCTTACATCAGCCCA |
| | | ATCGGATGCCTGCCCGCCCACCTGCTGGGCGACATGTGGG |
| | | GCCGCTTCTGGACAAACCTGTACTCCCTGACCGTGCCTTTT |
| | | GGCCAGAAGCCAAATATCGACGTGACCGATGCCATGGTGG |
| | | ACCAGGCCTGGGATGCCCAGCGGATCTTCAAGGAGGCCGA |
| | | GAAGTTCTTCGTGAGCGTGGGCCTGCCAAACATGACACAG |
| | | GGCTTTTGGGAGAACAGCATGCTGACCGACCCTGGCAACG |
| | | TGCAGAAGGCCGTGTGCCACCCAACAGCCTGGGACCTGGG |
| | | CAAGGGCGATTTCAGAATCCTGATGTGCACCAAGGTGACA |
| | | ATGGACGATTTTCTGACCGCCCACAACGAGATGGGCAACA |
| | | TCCAGTATGATATGGCATACGCAGCACAGCCCTTCCTGCTG |
| | | AGGAATGGAGCCAACGAGGGCTTTCACGAGGCCGTGGGCG |
| | | AGATCATGAGCCTGTCCGCCGCCACACCCAAGCACCTGAA |
| | | GTCTATCGGCCTGCTGAGCCCTGACTTCCAGGAGGATAACG |
| | | AGACCGAGATCAATTTTCTGCTGAAGCAGGCCCTGACCATC |
| | | GTGGGCACACTGCCTTTCACCTATATGCTGGAGAAGTGGA |
| | | GATGGATGGTGTTTAAGGGCGAGATCCCAAAGGACCAGTG |
| | | GATGAAGAAGTGGTGGGAGATGAAGAGGGAGATCGTGGG |
| | | AGTGGTGGAGCCCGTGCCTCACGACGAGACCTACTGTGAT |
| | | CCCGCCTCCCTGTTCCACGTGTCCAACGACTATTCTTTTATC |
| | | AGGTACTATACCCGCACACTGTACCAGTTCCAGTTTCAGGA |
| | | GGCCCTGTGCCAGGCAGCAAAGCACGAGGGCCCACTGCAC |
| | | AAGTGTGATATCTCTAACAGCACAGAGGCCGGACAGAAGC |
| | | TGTTCAATATGCTGCGCCTGGGCAAGTCTGAGCCCTGGACC |
| | | CTGGCCCTGGAGAACGTGGTGGGAGCCAAGAATATGAACG |
| | | TGCGGCCCCTGCTGAATTATTTCGAGCCTCTGTTTACATGG |
| | | CTGAAGGATCAGAATAAGAACAGCTTTGTGGGATGGAGCA |
| | | CCGACTGGTCCCCATACGCCGATCAGTCCATCAAGGTGCGC |
| | | ATCTCCCTGAAGTCTGCCCTGGGCGACAAGGCCTATGAGTG |
| | | GAATGATAACGAGATGTACCTGTTCAGATCCTCTGTGGCCT |
| | | ATGCCATGAGGCAGTACTTCCTGAAGGTGAAGAACCAGAT |
| | | GATCCTGTTTGGCGAGGAGGACGTGCGGGTGGCCAATCTG |
| | | AAGCCTAGAATCTCTTTTAACTTCTTTGTGACAGCCCCAAA |
| | | GAATGTGAGCGATATCATCCCCAGAACCGAGGTGGAGAAG |
| | | GCCATCCGGATGTCCCGGTCTAGAATCAACGACGCCTTCCG |
| | | GCTGAATGATAACTCCCTGGAGTTTCTGGGCATCCAGCCCA |
| | | CCCTGGGCCCACCTAATCAGCCACCCGTGAGCGTGATCGC |
| | | AGAGCTGCCTCCAAAGGTGTCCGTGTTCGTGCCTCCTAGGG |
| | | ACGGCTTCTTTGGCAATCCACGCAAGAGCAAGCTGATCTGT |
| | | CAGGCCACCGGCTTTTCTCCCAGACAGATCCAGGTGAGCTG |
| | | GCTGAGAGAGGGCAAGCAAGTGGGCTCCGGAGTGACCACA |
| | | GATCAGGTGCAGGCAGAGGCAAAGGAGTCTGGACCAACCA |
| | | CATACAAGGTGACCTCCACACTGACCATCAAGGAGTCTGA |
| | | CTGGCTGAGCCAGTCCATGTTCACCTGCCGGGTGGATCACA |
| | | GAGGCCTGACATTTCAGCAGAACGCCAGCTCCATGTGCGT |
| | | GCCTGACCAGGATACCGCCATCAGAGTGTTCGCGATCCCTC |
| | | CATCCTTCGCCTCTATCTTTCTGACAAAGTCCACCAAGCTG |
| | | ACATGCCTGGTGACCGACCTGACCACATATGATTCCGTGAC |
| | | CATCTCTTGGACAAGGCAGAACGGCGAGGCCGTGAAGACA |
| | | CACACCAACATCTCTGAGAGCCACCCCAATGCCACCTTCAG |
| | | CGCCGTGGGAGAGGCATCCATCTGCGAGGACGATTGGAAT |
| | | AGCGGCGAGCGGTTTACATGTACCGTGACACACACCGACC |
| | | TGCCTAGCCCACTGAAGCAGACCATCTCCAGGCCTAAGGG |
| | | AGTGGCCCTGCACCGGCCGGACGTGTACCTGCTGCCCCCTG |
| | | CCAGGGAGCAGCTGAACCTGAGAGAGAGCGCCACAATCAC |
| | | CTGTCTGGTGACCGGCTTCTCCCCTGCCGACGTGTTTGTGC |
| | | AGTGGATGCAGAGGGGACAGCCCCTGTCTCCTGAGAAGTA |
| | | CGTGACAAGCGCCCCCATGCCTGAGCCACAGGCCCCAGGC |
| | | CGGTATTTCGCCCACTCTATCCTGACCGTGAGCGAGGAGGA |
| | | GTGGAACACAGGCGAGACATATACCTGCGTGGTGGCACAC |
| | | GAGGCCCTGCCTAATAGGGTGACCGAGCGCACAGTGGACA |
| | | AGAGCACCGGTAAGCCAACACTGTACAATGTGAGCCTGGT |
| | | CATGTCCGATACCGCCGGGACCTGTTAT |
| 12155 | 32 | EPKSSDKTHTCPPCPAPEAAGGPSVFLFPPKPKDTLMISRTPEV |
| | | TCVVVSVSHEDPEVKFNWYVDGVEVHNAKTKPREEQYNSTY |
| | | RVVSVLTVLHQDWLNGKEYKCKVSNKALPAPIEKTISKAKGQ |
| | | PREPQVYVYPPSRDELTKNQVSLTCLVKGFYPSDIAVEWESN |
| | | GQPENNYKTTPPVLDSDGSFALVSKLTVDKSRWQQGNVFSCS |
| | | VMHEALHNHYTQKSLSLSPG |
| | 33 | GAGCCAAAGAGCTCCGACAAGACCCACACATGCCCCCCTT |
| | | GTCCGGCGCCAGAGGCTGCAGGAGGACCAAGCGTGTTCCT |
| | | GTTTCCACCCAAGCCTAAAGACACACTGATGATTTCCCGAA |
| | | CCCCCGAAGTCACATGCGTGGTCGTGTCTGTGAGTCACGAG |
| | | GACCCTGAAGTCAAGTTCAACTGGTACGTGGATGGCGTCG |
| | | AGGTGCATAATGCCAAGACTAAACCTAGGGAGGAACAGTA |
| | | CAACTCAACCTATCGCGTCGTGAGCGTCCTGACAGTGCTGC |
| | | ACCAGGATTGGCTGAACGGCAAAGAATATAAGTGCAAAGT |
| | | GAGCAATAAGGCCCTGCCCGCTCCTATCGAGAAAACCATT |
| | | TCCAAGGCTAAAGGGCAGCCTCGCGAACCACAGGTCTACG |
| | | TGTATCCTCCAAGCCGGGACGAGCTGACAAAGAACCAGGT |
| | | CTCCCTGACTTGTCTGGTGAAAGGGTTTTACCCTAGTGATA |
| | | TCGCTGTGGAGTGGGAATCAAATGGACAGCCAGAGAACAA |
| | | TTATAAGACTACCCCCCCTGTGCTGGACAGTGATGGGTCAT |
| | | TCGCACTGGTCTCCAAGCTGACAGTGGACAAATCTCGGTG |
| | | GCAGCAGGGAAATGTCTTTTCATGTAGCGTGATGCATGAA |
| | | GCACTGCACAACCATTACACCCAGAAGTCACTGTCACTGTC |
| | | ACCAGGA |
| 27618 | 34 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHHEMGHIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSEPKSSDKTHTCPPCPAPEAAGGPSVFLF |
| | | PPKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPG |
| | 35 | CAGTCCACAATCGAGGAGCAGGCCAAGACCTTCCTGGACA |
| | | AGTTTAACCACGAGGCCGAGGATCTGTTCTATCAGAGCTCC |
| | | CTGGCCAGCTGGAACTACAATACCAACATCACAGAGGAGA |
| | | ATGTGCAGAACATGAACAATGCCGGGGACAAGTGGTCCGC |
| | | CTTTCTGAAGGAGCAGTCTACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGTCTGAGGAT |
| | | AAGAGCAAGCGGCTGAATACAATCCTGAACACCATGTCCA |
| | | CAATCTATTCTACCGGCAAGGTGTGCAATCCCGACAACCCT |
| | | CAGGAGTGTCTGCTGCTGGAGCCTGGCCTGAATGAGATCA |
| | | TGGCCAACAGCCTGGATTACAATGAGAGGCTGTGGGCATG |
| | | GGAGAGCTGGCGCTCCGAAGTGGGCAAGCAGCTGCGGCCA |
| | | CTGTACGAGGAGTATGTGGTGCTGAAGAACGAGATGGCCA |
| | | GAGCCAATCACTACGAGGACTATGGCGATTACTGGAGGGG |
| | | CGACTACGAGGTGAACGGCGTGGACGGCTACGATTATTCC |
| | | AGAGGCCAGCTGATCGAGGATGTGGAGCACACATTCGAGG |
| | | AGATCAAGCCACTGTATGAGCACCTGCACGCCTACGTGAG |
| | | GGCCAAGCTGATGAACGCCTATCCTTCTTACATCAGCCCAA |
| | | TCGGATGCCTGCCCGCCCACCTGCTGGGCGACATGTGGGG |
| | | CCGCTTCTGGACAAACCTGTACAGCCTGACCGTGCCTTTTG |
| | | GCCAGAAGCCAAATATCGACGTGACCGATGCCATGGTGGA |
| | | CCAGGCCTGGGATGCCCAGCGGATCTTCAAGGAGGCCGAG |
| | | AAGTTCTTCGTGAGCGTGGGCCTGCCAAACATGACACAGG |
| | | GCTTTTGGGAGAACAGCATGCTGACCGACCCCGGCAACGT |
| | | GCAGAAGGCCGTGTGCCACCCTACAGCCTGGGACCTGGGC |
| | | AAGGGCGATTTCAGAATCCTGATGTGCACCAAGGTGACAA |
| | | TGGACGATTTTCTGACCGCCCACCACGAGATGGGCCACATC |
| | | CAGTATGATATGGCATACGCAGCACAGCCCTTCCTGCTGAG |
| | | GAATGGAGCCAACGAGGGCTTTCACGAGGCCGTGGGCGAG |
| | | ATCATGTCTCTGAGCGCCGCCACACCAAAGCACCTGAAGT |
| | | CCATCGGCCTGCTGTCTCCCGACTTCCAGGAGGATAACGAG |
| | | ACCGAGATCAATTTTCTGCTGAAGCAGGCCCTGACCATCGT |
| | | GGGCACACTGCCCTTCACCTATATGCTGGAGAAGTGGAGA |
| | | TGGATGGTGTTTAAGGGCGAGATCCCTAAGGACCAGTGGA |
| | | TGAAGAAGTGGTGGGAGATGAAGAGGGAGATCGTGGGAG |
| | | TGGTGGAGCCCGTGCCTCACGACGAGACCTACTGTGATCCC |
| | | GCCTCCCTGTTCCACGTGAGCAACGACTATTCCTTTATCAG |
| | | GTACTATACCCGCACACTGTACCAGTTCCAGTTTCAGGAGG |
| | | CCCTGTGCCAGGCAGCAAAGCACGAGGGCCCACTGCACAA |
| | | GTGTGATATCTCCAACTCTACAGAGGCCGGACAGAAGCTG |
| | | TTCAATATGCTGCGCCTGGGCAAGAGCGAGCCATGGACCC |
| | | TGGCCCTGGAGAACGTGGTGGGAGCCAAGAATATGAACGT |
| | | GCGGCCCCTGCTGAATTATTTCGAGCCTCTGTTTACATGGC |
| | | TGAAGGATCAGAATAAGAACAGCTTTGTGGGCTGGTCTAC |
| | | CGACTGGAGCCCCTACGCCGATCAGTCCATCAAGGTGAGA |
| | | ATCTCCCTGAAGTCTGCCCTGGGCGACAAGGCCTATGAGTG |
| | | GAATGATAACGAGATGTACCTGTTCAGATCCTCTGTGGCCT |
| | | ATGCCATGAGGCAGTACTTCCTGAAGGTGAAGAACCAGAT |
| | | GATCCTGTTTGGCGAGGAGGACGTGCGGGTGGCCAATCTG |
| | | AAGCCTAGAATCAGCTTTAACTTCTTTGTGACAGCCCCAAA |
| | | GAACGTGAGCGATATCATCCCCAGAACCGAGGTGGAGAAG |
| | | GCCATCCGGATGAGCCGGTCCAGAATCAACGACGCCTTCC |
| | | GCCTGAATGATAACTCCCTGGAGTTTCTGGGCATCCAGCCT |
| | | ACCCTGGGCCCACCTAATCAGCCACCCGTGAGCGAGCCAA |
| | | AGAGCTCCGACAAGACCCACACATGCCCTCCATGTCCGGC |
| | | GCCAGAGGCCGCCGGAGGACCTAGCGTGTTCCTGTTTCCCC |
| | | CTAAGCCAAAGGATACACTGATGATCAGCCGGACACCAGA |
| | | GGTGACCTGCGTGGTGGTGTCTGTGAGCCACGAGGACCCC |
| | | GAGGTGAAGTTCAACTGGTATGTGGATGGCGTGGAGGTGC |
| | | ACAATGCCAAGACCAAGCCTAGAGAGGAGCAGTATAACAG |
| | | CACATACAGGGTGGTGTCCGTGCTGACCGTGCTGCACCAG |
| | | GACTGGCTGAACGGCAAGGAGTACAAGTGCAAGGTGTCTA |
| | | ATAAGGCCCTGCCTGCCCCAATCGAGAAGACCATCAGCAA |
| | | GGCAAAGGGACAGCCTCGCGAACCACAGGTGTACACTCTG |
| | | CCTCCATCTCGGGACGAGCTGACTAAGAACCAGGTCAGTC |
| | | TGACCTGTCTGGTGAAAGGATTCTATCCCAGCGATATCGCT |
| | | GTGGAGTGGGAATCCAATGGCCAGCCTGAGAACAATTACA |
| | | AGACCACACCCCCTGTGCTGGACTCTGATGGCAGTTTCTTT |
| | | CTGTATAGTAAGCTGACCGTCGATAAATCACGATGGCAGC |
| | | AGGGGAACGTGTTCAGCTGTTCAGTGATGCACGAAGCCCT |
| | | GCACAACCATTACACCCAGAAGAGCCTGAGCCTGTCTCCC |
| | | GGC |
| 27620 | 36 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSEPKSSDKTHTCPPCPAPEAAGGPSVFLF |
| | | PPKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPG |
| | 37 | CAGTCCACAATCGAGGAGCAGGCCAAGACCTTCCTGGACA |
| | | AGTTTAACCACGAGGCCGAGGATCTGTTCTATCAGAGCTCC |
| | | CTGGCCTCTTGGAACTACAATACCAACATCACAGAGGAGA |
| | | ATGTGCAGAACATGAACAATGCCGGGGACAAGTGGAGCGC |
| | | CTTTCTGAAGGAGCAGTCCACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGTCTGAGGAT |
| | | AAGAGCAAGAGGCTGAATACAATCCTGAACACCATGTCCA |
| | | CAATCTATTCTACCGGCAAGGTGTGCAATCCAGACAACCCC |
| | | CAGGAGTGTCTGCTGCTGGAGCCTGGCCTGAATGAGATCA |
| | | TGGCCAACAGCCTGGATTACAATGAGAGGCTGTGGGCATG |
| | | GGAGTCTTGGCGCAGCGAAGTGGGCAAGCAGCTGCGGCCA |
| | | CTGTACGAGGAGTATGTGGTGCTGAAGAACGAGATGGCCA |
| | | GAGCCAATCACTACGAGGACTATGGCGATTACTGGAGGGG |
| | | CGACTACGAGGTGAACGGCGTGGACGGCTACGATTATAGC |
| | | AGAGGCCAGCTGATCGAGGATGTGGAGCACACATTCGAGG |
| | | AGATCAAGCCCCTGTATGAGCACCTGCACGCCTACGTGAG |
| | | GGCCAAGCTGATGAACGCCTATCCTTCCTACATCTCTCCAA |
| | | TCGGATGCCTGCCCGCCCACCTGCTGGGCGACATGTGGGG |
| | | CCGCTTCTGGACAAACCTGTACTCCCTGACCGTGCCTTTTG |
| | | GCCAGAAGCCAAATATCGACGTGACCGATGCCATGGTGGA |
| | | CCAGGCCTGGGATGCCCAGCGGATCTTCAAGGAGGCCGAG |
| | | AAGTTCTTCGTGAGCGTGGGCCTGCCAAACATGACACAGG |
| | | GCTTTTGGGAGAACAGCATGCTGACCGACCCTGGCAACGT |
| | | GCAGAAGGCCGTGTGCCACCCAACAGCCTGGGACCTGGGC |
| | | AAGGGCGATTTCAGAATCCTGATGTGCACCAAGGTGACAA |
| | | TGGACGATTTTCTGACCGCCCACAATGAGATGGGCAACAT |
| | | CCAGTATGATATGGCATACGCAGCACAGCCCTTCCTGCTGA |
| | | GGAATGGAGCCAACGAGGGCTTTCACGAGGCCGTGGGCGA |
| | | GATCATGTCTCTGAGCGCCGCCACACCCAAGCACCTGAAG |
| | | TCCATCGGCCTGCTGTCTCCTGACTTCCAGGAGGATAACGA |
| | | GACCGAGATCAATTTTCTGCTGAAGCAGGCCCTGACCATCG |
| | | TGGGCACACTGCCCTTCACCTATATGCTGGAGAAGTGGAG |
| | | ATGGATGGTGTTTAAGGGCGAGATCCCTAAGGACCAGTGG |
| | | ATGAAGAAGTGGTGGGAGATGAAGAGGGAGATCGTGGGA |
| | | GTGGTGGAGCCCGTGCCTCACGACGAGACCTACTGTGATC |
| | | CCGCCAGCCTGTTCCACGTGAGCAACGACTATTCCTTTATC |
| | | AGGTACTATACCCGCACACTGTACCAGTTCCAGTTTCAGGA |
| | | GGCCCTGTGCCAGGCAGCAAAGCACGAGGGCCCACTGCAC |
| | | AAGTGTGATATCTCCAACTCTACAGAGGCCGGACAGAAGC |
| | | TGTTCAATATGCTGCGCCTGGGCAAGTCCGAGCCATGGACC |
| | | CTGGCCCTGGAGAACGTGGTGGGAGCCAAGAATATGAACG |
| | | TGCGGCCCCTGCTGAATTATTTCGAGCCTCTGTTTACATGG |
| | | CTGAAGGATCAGAATAAGAACAGCTTTGTGGGCTGGTCTA |
| | | CCGACTGGAGCCCCTACGCCGATCAGTCCATCAAGGTGCG |
| | | CATCAGCCTGAAGTCCGCCCTGGGCGACAAGGCCTATGAG |
| | | TGGAATGATAACGAGATGTACCTGTTCAGATCCTCTGTGGC |
| | | CTATGCCATGAGGCAGTACTTCCTGAAGGTGAAGAACCAG |
| | | ATGATCCTGTTTGGCGAGGAGGACGTGCGGGTGGCCAATC |
| | | TGAAGCCTAGAATCTCTTTTAACTTCTTTGTGACAGCCCCA |
| | | AAGAATGTGAGCGATATCATCCCCAGAACCGAGGTGGAGA |
| | | AGGCCATCCGGATGAGCCGGTCCAGAATCAACGACGCCTT |
| | | CCGGCTGAATGATAACTCTCTGGAGTTTCTGGGCATCCAGC |
| | | CAACCCTGGGCCCACCTAATCAGCCACCCGTGAGCGAGCC |
| | | CAAGAGCTCCGACAAGACCCACACATGCCCTCCATGTCCG |
| | | GCGCCAGAGGCCGCCGGAGGACCAAGCGTGTTCCTGTTTC |
| | | CACCCAAGCCCAAGGACACCCTGATGATCTCCCGGACCCC |
| | | AGAGGTGACATGCGTGGTGGTGAGCGTGTCCCACGAGGAC |
| | | CCCGAGGTGAAGTTTAACTGGTACGTGGATGGCGTGGAGG |
| | | TGCACAATGCCAAGACAAAGCCCCGGGAGGAGCAGTACAA |
| | | TTCTACCTATAGAGTGGTGAGCGTGCTGACAGTGCTGCACC |
| | | AGGATTGGCTGAACGGCAAGGAGTATAAGTGTAAGGTGAG |
| | | CAATAAGGCCCTGCCAGCCCCCATCGAGAAGACCATCTCC |
| | | AAGGCCAAGGGCCAGCCTCGCGAACCACAGGTGTACACTC |
| | | TGCCTCCATCTCGGGACGAGCTGACTAAGAACCAGGTCAG |
| | | TCTGACCTGTCTGGTGAAAGGATTCTATCCCAGCGATATCG |
| | | CTGTGGAGTGGGAATCCAATGGCCAGCCTGAGAACAATTA |
| | | CAAGACCACACCCCCTGTGCTGGACTCTGATGGCAGTTTCT |
| | | TTCTGTATAGTAAGCTGACCGTCGATAAATCACGATGGCAG |
| | | CAGGGGAACGTGTTCAGCTGTTCAGTGATGCACGAAGCCC |
| | | TGCACAACCATTACACCCAGAAGAGCCTGAGCCTGTCTCCC |
| | | GGC |
| 27621 | 38 | QSTIEEQAKTFLDKFNHEAEDLFYQSSLASWNYNTNITEENVQ |
| | | NMNNAGDKWSAFLKEQSTLAQMYPLQEIQNLTVKLQLQALQ |
| | | QNGSSVLSEDKSKRLNTILNTMSTIYSTGKVCNPDNPQECLLL |
| | | EPGLNEIMANSLDYNERLWAWESWRSEVGKQLRPLYEEYVV |
| | | LKNEMARANHYEDYGDYWRGDYEVNGVDGYDYSRGQLIED |
| | | VEHTFEEIKPLYEHLHAYVRAKLMNAYPSYISPIGCLPAHLLG |
| | | DMWGRFWTNLYSLTVPFGQKPNIDVTDAMVDQAWDAQRIF |
| | | KEAEKFFVSVGLPNMTQGFWENSMLTDPGNVQKAVCHPTA |
| | | WDLGKGDFRILMCTKVTMDDFLTAHNEMGNIQYDMAYAAQ |
| | | PFLLRNGANEGFHEAVGEIMSLSAATPKHLKSIGLLSPDFQED |
| | | NETEINFLLKQALTIVGTLPFTYMLEKWRWMVFKGEIPKDQW |
| | | MKKWWEMKREIVGVVEPVPHDETYCDPASLFHVSNDYSFIR |
| | | YYTRTLYQFQFQEALCQAAKHEGPLHKCDISNSTEAGQKLFN |
| | | MLRLGKSEPWTLALENVVGAKNMNVRPLLNYFEPLFTWLKD |
| | | QNKNSFVGWSTDWSPYADQSIKVRISLKSALGDKAYEWNDN |
| | | EMYLFRSSVAYAMRQYFLKVKNQMILFGEEDVRVANLKPRIS |
| | | FNFFVTAPKNVSDIIPRTEVEKAIRMSRSRINDAFRLNDNSLEF |
| | | LGIQPTLGPPNQPPVSEPKSSDKTHTCPPCPAPEAAGGPSVFLF |
| | | PPKPKDTLMISRTPEVTCVVVSVSHEDPEVKFNWYVDGVEVH |
| | | NAKTKPREEQYNSTYRVVSVLTVLHQDWLNGKEYKCKVSN |
| | | KALPAPIEKTISKAKGQPREPQVYTLPPSRDELTKNQVSLTCL |
| | | VKGFYPSDIAVEWESNGQPENNYKTTPPVLDSDGSFFLYSKLT |
| | | VDKSRWQQGNVFSCSVMHEALHNHYTQKSLSLSPGGGGGSG |
| | | GGGSGGGGSQSTIEEQAKTFLDKFNHEAEDLFYQSSLASWNY |
| | | NTNITEENVQNMNNAGDKWSAFLKEQSTLAQMYPLQEIQNL |
| | | TVKLQLQALQQNGSSVLSEDKSKRLNTILNTMSTIYSTGKVC |
| | | NPDNPQECLLLEPGLNEIMANSLDYNERLWAWESWRSEVGK |
| | | QLRPLYEEYVVLKNEMARANHYEDYGDYWRGDYEVNGVD |
| | | GYDYSRGQLIEDVEHTFEEIKPLYEHLHAYVRAKLMNAYPSY |
| | | ISPIGCLPAHLLGDMWGRFWTNLYSLTVPFGQKPNIDVTDAM |
| | | VDQAWDAQRIFKEAEKFFVSVGLPNMTQGFWENSMLTDPGN |
| | | VQKAVCHPTAWDLGKGDFRILMCTKVTMDDFLTAHNEMGN |
| | | IQYDMAYAAQPFLLRNGANEGFHEAVGEIMSLSAATPKHLKS |
| | | IGLLSPDFQEDNETEINFLLKQALTIVGTLPFTYMLEKWRWMV |
| | | FKGEIPKDQWMKKWWEMKREIVGVVEPVPHDETYCDPASLF |
| | | HVSNDYSFIRYYTRTLYQFQFQEALCQAAKHEGPLHKCDISNS |
| | | TEAGQKLFNMLRLGKSEPWTLALENVVGAKNMNVRPLLNYF |
| | | EPLFTWLKDQNKNSFVGWSTDWSPYADQSIKVRISLKSALGD |
| | | KAYEWNDNEMYLFRSSVAYAMRQYFLKVKNQMILFGEEDV |
| | | RVANLKPRISFNFFVTAPKNVSDIIPRTEVEKAIRMSRSRINDA |
| | | FRLNDNSLEFLGIQPTLGPPNQPPVS |
| | 39 | CAGTCCACAATCGAGGAGCAGGCCAAGACCTTCCTGGACA |
| | | AGTTTAACCACGAGGCCGAGGATCTGTTCTATCAGAGCTCC |
| | | CTGGCCTCTTGGAACTACAATACCAACATCACAGAGGAGA |
| | | ATGTGCAGAACATGAACAATGCCGGGGACAAGTGGAGCGC |
| | | CTTTCTGAAGGAGCAGTCCACACTGGCCCAGATGTACCCTC |
| | | TGCAGGAGATCCAGAACCTGACCGTGAAGCTGCAGCTGCA |
| | | GGCCCTGCAGCAGAATGGCTCTAGCGTGCTGTCTGAGGAT |
| | | AAGAGCAAGAGGCTGAATACAATCCTGAACACCATGTCCA |
| | | CAATCTATTCTACCGGCAAGGTGTGCAATCCAGACAACCCC |
| | | CAGGAGTGTCTGCTGCTGGAGCCTGGCCTGAATGAGATCA |
| | | TGGCCAACAGCCTGGATTACAATGAGAGGCTGTGGGCATG |
| | | GGAGTCTTGGCGCAGCGAAGTGGGCAAGCAGCTGCGGCCA |
| | | CTGTACGAGGAGTATGTGGTGCTGAAGAACGAGATGGCCA |
| | | GAGCCAATCACTACGAGGACTATGGCGATTACTGGAGGGG |
| | | CGACTACGAGGTGAACGGCGTGGACGGCTACGATTATAGC |
| | | AGAGGCCAGCTGATCGAGGATGTGGAGCACACATTCGAGG |
| | | AGATCAAGCCCCTGTATGAGCACCTGCACGCCTACGTGAG |
| | | GGCCAAGCTGATGAACGCCTATCCTTCCTACATCTCTCCAA |
| | | TCGGATGCCTGCCCGCCCACCTGCTGGGCGACATGTGGGG |
| | | CCGCTTCTGGACAAACCTGTACTCCCTGACCGTGCCTTTTG |
| | | GCCAGAAGCCAAATATCGACGTGACCGATGCCATGGTGGA |
| | | CCAGGCCTGGGATGCCCAGCGGATCTTCAAGGAGGCCGAG |
| | | AAGTTCTTCGTGAGCGTGGGCCTGCCAAACATGACACAGG |
| | | GCTTTTGGGAGAACAGCATGCTGACCGACCCTGGCAACGT |
| | | GCAGAAGGCCGTGTGCCACCCAACAGCCTGGGACCTGGGC |
| | | AAGGGCGATTTCAGAATCCTGATGTGCACCAAGGTGACAA |
| | | TGGACGATTTTCTGACCGCCCACAATGAGATGGGCAACAT |
| | | CCAGTATGATATGGCATACGCAGCACAGCCCTTCCTGCTGA |
| | | GGAATGGAGCCAACGAGGGCTTTCACGAGGCCGTGGGCGA |
| | | GATCATGTCTCTGAGCGCCGCCACACCCAAGCACCTGAAG |
| | | TCCATCGGCCTGCTGTCTCCTGACTTCCAGGAGGATAACGA |
| | | GACCGAGATCAATTTTCTGCTGAAGCAGGCCCTGACCATCG |
| | | TGGGCACACTGCCCTTCACCTATATGCTGGAGAAGTGGAG |
| | | ATGGATGGTGTTTAAGGGCGAGATCCCTAAGGACCAGTGG |
| | | ATGAAGAAGTGGTGGGAGATGAAGAGGGAGATCGTGGGA |
| | | GTGGTGGAGCCCGTGCCTCACGACGAGACCTACTGTGATC |
| | | CCGCCAGCCTGTTCCACGTGAGCAACGACTATTCCTTTATC |
| | | AGGTACTATACCCGCACACTGTACCAGTTCCAGTTTCAGGA |
| | | GGCCCTGTGCCAGGCAGCAAAGCACGAGGGCCCACTGCAC |
| | | AAGTGTGATATCTCCAACTCTACAGAGGCCGGACAGAAGC |
| | | TGTTCAATATGCTGCGCCTGGGCAAGTCCGAGCCATGGACC |
| | | CTGGCCCTGGAGAACGTGGTGGGAGCCAAGAATATGAACG |
| | | TGCGGCCCCTGCTGAATTATTTCGAGCCTCTGTTTACATGG |
| | | CTGAAGGATCAGAATAAGAACAGCTTTGTGGGCTGGTCTA |
| | | CCGACTGGAGCCCCTACGCCGATCAGTCCATCAAGGTGCG |
| | | CATCAGCCTGAAGTCCGCCCTGGGCGACAAGGCCTATGAG |
| | | TGGAATGATAACGAGATGTACCTGTTCAGATCCTCTGTGGC |
| | | CTATGCCATGAGGCAGTACTTCCTGAAGGTGAAGAACCAG |
| | | ATGATCCTGTTTGGCGAGGAGGACGTGCGGGTGGCCAATC |
| | | TGAAGCCTAGAATCTCTTTTAACTTCTTTGTGACAGCCCCA |
| | | AAGAATGTGAGCGATATCATCCCCAGAACCGAGGTGGAGA |
| | | AGGCCATCCGGATGAGCCGGTCCAGAATCAACGACGCCTT |
| | | CCGGCTGAATGATAACTCTCTGGAGTTTCTGGGCATCCAGC |
| | | CAACCCTGGGCCCACCTAATCAGCCACCCGTGAGCGAGCC |
| | | CAAGAGCTCCGACAAGACCCACACATGCCCTCCATGTCCG |
| | | GCGCCAGAGGCCGCCGGAGGACCAAGCGTGTTCCTGTTTC |
| | | CACCCAAGCCCAAGGACACCCTGATGATCTCCCGGACCCC |
| | | AGAGGTGACATGCGTGGTGGTGAGCGTGTCCCACGAGGAC |
| | | CCCGAGGTGAAGTTTAACTGGTACGTGGATGGCGTGGAGG |
| | | TGCACAATGCCAAGACAAAGCCCCGGGAGGAGCAGTACAA |
| | | TTCTACCTATAGAGTGGTGAGCGTGCTGACAGTGCTGCACC |
| | | AGGATTGGCTGAACGGCAAGGAGTATAAGTGTAAGGTGAG |
| | | CAATAAGGCCCTGCCAGCCCCCATCGAGAAGACCATCTCC |
| | | AAGGCCAAGGGCCAGCCTCGCGAGCCCCAGGTGTATACCC |
| | | TGCCCCCTAGCAGAGACGAGCTGACAAAGAACCAGGTGTC |
| | | CCTGACCTGCCTGGTGAAGGGCTTCTACCCTTCCGATATCG |
| | | CCGTGGAGTGGGAGTCTAATGGCCAGCCAGAGAACAATTA |
| | | TAAGACCACACCACCCGTGCTGGACTCCGATGGCTCTTTCT |
| | | TTCTGTACAGCAAGCTGACAGTGGACAAGTCCAGGTGGCA |
| | | GCAGGGCAACGTGTTCAGCTGTTCCGTGATGCACGAGGCC |
| | | CTGCACAATCACTACACCCAGAAGTCTCTGAGCTTAAGCCC |
| | | TGGAGGAGGAGGAGGCTCTGGAGGAGGAGGCAGCGGCGG |
| | | AGGAGGCTCTCAGAGCACAATCGAGGAGCAGGCCAAGACC |
| | | TTCCTGGACAAGTTTAACCACGAGGCCGAGGATCTGTTCTA |
| | | TCAGAGCTCCCTGGCCTCCTGGAACTACAATACCAACATCA |
| | | CAGAGGAGAATGTGCAGAACATGAACAATGCCGGGGACA |
| | | AGTGGTCTGCCTTTCTGAAGGAGCAGAGCACACTGGCCCA |
| | | GATGTACCCACTGCAGGAGATCCAGAACCTGACCGTGAAG |
| | | CTGCAGCTGCAGGCCCTGCAGCAGAATGGCTCTAGCGTGC |
| | | TGAGCGAGGATAAGTCCAAGAGGCTGAATACAATCCTGAA |
| | | CACCATGTCTACAATCTATAGCACCGGCAAGGTGTGCAATC |
| | | CTGACAACCCACAGGAGTGTCTGCTGCTGGAGCCTGGCCT |
| | | GAATGAGATCATGGCCAACAGCCTGGATTACAATGAGAGG |
| | | CTGTGGGCATGGGAGAGCTGGCGCTCCGAAGTGGGCAAGC |
| | | AGCTGCGGCCACTGTACGAGGAGTATGTGGTGCTGAAGAA |
| | | CGAGATGGCCAGAGCCAATCACTACGAGGACTATGGCGAT |
| | | TACTGGAGGGGCGACTATGAGGTGAACGGCGTGGACGGCT |
| | | ACGATTATAGCCGGGGCCAGCTGATCGAGGATGTGGAGCA |
| | | CACATTCGAGGAGATCAAGCCCCTGTATGAGCACCTGCAC |
| | | GCCTACGTGCGGGCCAAGCTGATGAACGCCTATCCCTCTTA |
| | | CATCAGCCCTATCGGATGCCTGCCAGCCCACCTGCTGGGCG |
| | | ACATGTGGGGCAGATTCTGGACAAACCTGTACAGCCTGAC |
| | | CGTGCCCTTTGGCCAGAAGCCTAATATCGACGTGACCGATG |
| | | CCATGGTGGACCAGGCCTGGGATGCCCAGAGGATCTTCAA |
| | | GGAGGCCGAGAAGTTCTTCGTGAGCGTGGGCCTGCCAAAC |
| | | ATGACACAGGGCTTTTGGGAGAACAGCATGCTGACCGACC |
| | | CCGGCAACGTGCAGAAGGCCGTGTGCCACCCTACAGCCTG |
| | | GGACCTGGGCAAGGGCGATTTCCGCATCCTGATGTGCACC |
| | | AAGGTGACAATGGACGATTTTCTGACCGCCCACAATGAGA |
| | | TGGGCAACATCCAGTATGATATGGCATACGCAGCACAGCC |
| | | CTTCCTGCTGAGGAATGGAGCCAACGAGGGCTTTCACGAG |
| | | GCCGTGGGCGAGATCATGTCCCTGTCTGCCGCCACACCAA |
| | | AGCACCTGAAGTCCATCGGCCTGCTGTCTCCCGACTTCCAG |
| | | GAGGATAACGAGACCGAGATCAATTTTCTGCTGAAGCAGG |
| | | CCCTGACCATCGTGGGCACACTGCCCTTCACCTATATGCTG |
| | | GAGAAGTGGCGCTGGATGGTGTTTAAGGGCGAGATCCCTA |
| | | AGGACCAGTGGATGAAGAAGTGGTGGGAGATGAAGCGGG |
| | | AGATCGTGGGAGTGGTGGAGCCAGTGCCCCACGACGAGAC |
| | | CTACTGTGATCCCGCCTCCCTGTTCCACGTGTCCAACGACT |
| | | ATTCTTTTATCAGGTACTATACCCGCACACTGTACCAGTTC |
| | | CAGTTTCAGGAGGCCCTGTGCCAGGCAGCAAAGCACGAGG |
| | | GCCCACTGCACAAGTGTGATATCAGCAACTCCACAGAGGC |
| | | CGGACAGAAGCTGTTCAATATGCTGAGACTGGGCAAGAGC |
| | | GAGCCATGGACCCTGGCCCTGGAGAACGTGGTGGGAGCCA |
| | | AGAATATGAACGTGAGGCCACTGCTGAATTATTTCGAGCC |
| | | CCTGTTTACATGGCTGAAGGATCAGAATAAGAACAGCTTT |
| | | GTGGGCTGGAGCACCGACTGGTCCCCTTACGCCGATCAGTC |
| | | CATCAAGGTGCGCATCTCCCTGAAGTCTGCCCTGGGCGACA |
| | | AGGCCTATGAGTGGAATGATAACGAGATGTACCTGTTCAG |
| | | ATCCTCTGTGGCCTATGCCATGAGGCAGTACTTCCTGAAGG |
| | | TGAAGAACCAGATGATCCTGTTTGGCGAGGAGGACGTGCG |
| | | GGTGGCCAATCTGAAGCCCAGAATCTCTTTTAACTTCTTTG |
| | | TGACAGCCCCTAAGAATGTGAGCGATATCATCCCACGCAC |
| | | CGAGGTGGAGAAGGCCATCCGGATGTCTCGGAGCAGAATC |
| | | AACGACGCCTTCCGGCTGAATGATAACTCTCTGGAGTTTCT |
| | | GGGCATCCAGCCTACCCTGGGCCCTCCAAATCAGCCCCCTG |
| | | TGAGC |
| 27656 | 40 | EPKSSDKTHTCPPCPAPEAAGGPSVFLFPPKPKDTLMISRTPEV |
| | | TCVVVSVSHEDPEVKFNWYVDGVEVHNAKTKPREEQYNSTY |
| | | RVVSVLTVLHQDWLNGKEYKCKVSNKALPAPIEKTISKAKGQ |
| | | PREPQVYTLPPSRDELTKNQVSLTCLVKGFYPSDIAVEWESNG |
| | | QPENNYKTTPPVLDSDGSFFLYSKLTVDKSRWQQGNVFSCSV |
| | | MHEALHNHYTQKSLSLSPGGGGGSGGGGSGGGGSQSTIEEQA |
| | | KTFLDKFNHEAEDLFYQSSLASWNYNTNITEENVQNMNNAG |
| | | DKWSAFLKEQSTLAQMYPLQEIQNLTVKLQLQALQQNGSSV |
| | | LSEDKSKRLNTILNTMSTIYSTGKVCNPDNPQECLLLEPGLNEI |
| | | MANSLDYNERLWAWESWRSEVGKQLRPLYEEYVVLKNEMA |
| | | RANHYEDYGDYWRGDYEVNGVDGYDYSRGQLIEDVEHTFE |
| | | EIKPLYEHLHAYVRAKLMNAYPSYISPIGCLPAHLLGDMWGR |
| | | FWTNLYSLTVPFGQKPNIDVTDAMVDQAWDAQRIFKEAEKF |
| | | FVSVGLPNMTQGFWENSMLTDPGNVQKAVCHPTAWDLGKG |
| | | DFRILMCTKVTMDDFLTAHNEMGNIQYDMAYAAQPFLLRNG |
| | | ANEGFHEAVGEIMSLSAATPKHLKSIGLLSPDFQEDNETEINFL |
| | | LKQALTIVGTLPFTYMLEKWRWMVFKGEIPKDQWMKKWWE |
| | | MKREIVGVVEPVPHDETYCDPASLFHVSNDYSFIRYYTRTLY |
| | | QFQFQEALCQAAKHEGPLHKCDISNSTEAGQKLFNMLRLGKS |
| | | EPWTLALENVVGAKNMNVRPLLNYFEPLFTWLKDQNKNSFV |
| | | GWSTDWSPYADQSIKVRISLKSALGDKAYEWNDNEMYLFRS |
| | | SVAYAMRQYFLKVKNQMILFGEEDVRVANLKPRISFNFFVTA |
| | | PKNVSDIIPRTEVEKAIRMSRSRINDAFRLNDNSLEFLGIQPTLG |
| | | PPNQPPVS |
| | 41 | GAGCCAAAGAGCTCCGACAAGACCCACACATGCCCCCCTT |
| | | GTCCGGCGCCAGAGGCAGCAGGAGGACCAAGCGTGTTCCT |
| | | GTTTCCACCCAAGCCCAAAGACACCCTGATGATTAGCCGA |
| | | ACCCCTGAAGTCACATGCGTGGTCGTGTCCGTGTCTCACGA |
| | | GGACCCAGAAGTCAAGTTCAACTGGTACGTGGATGGCGTC |
| | | GAGGTGCATAATGCCAAGACAAAACCCCGGGAGGAACAGT |
| | | ACAACAGCACCTATAGAGTCGTGTCCGTCCTGACAGTGCTG |
| | | CACCAGGATTGGCTGAACGGCAAGGAATATAAGTGCAAAG |
| | | TGTCCAATAAGGCCCTGCCCGCTCCTATCGAGAAAACCATT |
| | | TCTAAGGCAAAAGGCCAGCCTCGCGAGCCCCAGGTGTATA |
| | | CCCTGCCCCCTAGCAGAGACGAGCTGACAAAGAACCAGGT |
| | | GTCCCTGACCTGCCTGGTGAAGGGCTTCTACCCTTCCGATA |
| | | TCGCCGTGGAGTGGGAGTCTAATGGCCAGCCAGAGAACAA |
| | | TTATAAGACCACACCACCCGTGCTGGACTCCGATGGCTCTT |
| | | TCTTTCTGTACAGCAAGCTGACAGTGGACAAGTCCAGGTG |
| | | GCAGCAGGGCAACGTGTTCAGCTGTTCCGTGATGCACGAG |
| | | GCCCTGCACAATCACTACACCCAGAAGTCTCTGAGCTTAAG |
| | | CCCTGGAGGAGGAGGAGGCTCTGGAGGAGGAGGCAGCGG |
| | | CGGAGGAGGCTCTCAGAGCACAATCGAGGAGCAGGCCAA |
| | | GACCTTCCTGGACAAGTTTAACCACGAGGCCGAGGATCTG |
| | | TTCTATCAGAGCTCCCTGGCCTCCTGGAACTACAATACCAA |
| | | CATCACAGAGGAGAATGTGCAGAACATGAACAATGCCGGG |
| | | GACAAGTGGTCTGCCTTTCTGAAGGAGCAGAGCACACTGG |
| | | CCCAGATGTACCCACTGCAGGAGATCCAGAACCTGACCGT |
| | | GAAGCTGCAGCTGCAGGCCCTGCAGCAGAATGGCTCTAGC |
| | | GTGCTGAGCGAGGATAAGTCCAAGAGGCTGAATACAATCC |
| | | TGAACACCATGTCTACAATCTATAGCACCGGCAAGGTGTG |
| | | CAATCCTGACAACCCACAGGAGTGTCTGCTGCTGGAGCCT |
| | | GGCCTGAATGAGATCATGGCCAACAGCCTGGATTACAATG |
| | | AGAGGCTGTGGGCATGGGAGAGCTGGCGCTCCGAAGTGGG |
| | | CAAGCAGCTGCGGCCACTGTACGAGGAGTATGTGGTGCTG |
| | | AAGAACGAGATGGCCAGAGCCAATCACTACGAGGACTATG |
| | | GCGATTACTGGAGGGGCGACTATGAGGTGAACGGCGTGGA |
| | | CGGCTACGATTATAGCCGGGGCCAGCTGATCGAGGATGTG |
| | | GAGCACACATTCGAGGAGATCAAGCCCCTGTATGAGCACC |
| | | TGCACGCCTACGTGCGGGCCAAGCTGATGAACGCCTATCC |
| | | CTCTTACATCAGCCCTATCGGATGCCTGCCAGCCCACCTGC |
| | | TGGGCGACATGTGGGGCAGATTCTGGACAAACCTGTACAG |
| | | CCTGACCGTGCCCTTTGGCCAGAAGCCTAATATCGACGTGA |
| | | CCGATGCCATGGTGGACCAGGCCTGGGATGCCCAGAGGAT |
| | | CTTCAAGGAGGCCGAGAAGTTCTTCGTGAGCGTGGGCCTG |
| | | CCAAACATGACACAGGGCTTTTGGGAGAACAGCATGCTGA |
| | | CCGACCCCGGCAACGTGCAGAAGGCCGTGTGCCACCCTAC |
| | | AGCCTGGGACCTGGGCAAGGGCGATTTCCGCATCCTGATG |
| | | TGCACCAAGGTGACAATGGACGATTTTCTGACCGCCCACA |
| | | ATGAGATGGGCAACATCCAGTATGATATGGCATACGCAGC |
| | | ACAGCCCTTCCTGCTGAGGAATGGAGCCAACGAGGGCTTT |
| | | CACGAGGCCGTGGGCGAGATCATGTCCCTGTCTGCCGCCA |
| | | CACCAAAGCACCTGAAGTCCATCGGCCTGCTGTCTCCCGAC |
| | | TTCCAGGAGGATAACGAGACCGAGATCAATTTTCTGCTGA |
| | | AGCAGGCCCTGACCATCGTGGGCACACTGCCCTTCACCTAT |
| | | ATGCTGGAGAAGTGGCGCTGGATGGTGTTTAAGGGCGAGA |
| | | TCCCTAAGGACCAGTGGATGAAGAAGTGGTGGGAGATGAA |
| | | GCGGGAGATCGTGGGAGTGGTGGAGCCAGTGCCCCACGAC |
| | | GAGACCTACTGTGATCCCGCCTCCCTGTTCCACGTGTCCAA |
| | | CGACTATTCTTTTATCAGGTACTATACCCGCACACTGTACC |
| | | AGTTCCAGTTTCAGGAGGCCCTGTGCCAGGCAGCAAAGCA |
| | | CGAGGGCCCACTGCACAAGTGTGATATCAGCAACTCCACA |
| | | GAGGCCGGACAGAAGCTGTTCAATATGCTGAGACTGGGCA |
| | | AGAGCGAGCCATGGACCCTGGCCCTGGAGAACGTGGTGGG |
| | | AGCCAAGAATATGAACGTGAGGCCACTGCTGAATTATTTC |
| | | GAGCCCCTGTTTACATGGCTGAAGGATCAGAATAAGAACA |
| | | GCTTTGTGGGCTGGAGCACCGACTGGTCCCCTTACGCCGAT |
| | | CAGTCCATCAAGGTGCGCATCTCCCTGAAGTCTGCCCTGGG |
| | | CGACAAGGCCTATGAGTGGAATGATAACGAGATGTACCTG |
| | | TTCAGATCCTCTGTGGCCTATGCCATGAGGCAGTACTTCCT |
| | | GAAGGTGAAGAACCAGATGATCCTGTTTGGCGAGGAGGAC |
| | | GTGCGGGTGGCCAATCTGAAGCCCAGAATCTCTTTTAACTT |
| | | CTTTGTGACAGCCCCTAAGAATGTGAGCGATATCATCCCAC |
| | | GCACCGAGGTGGAGAAGGCCATCCGGATGTCTCGGAGCAG |
| | | AATCAACGACGCCTTCCGGCTGAATGATAACTCTCTGGAGT |
| | | TTCTGGGCATCCAGCCTACCCTGGGCCCTCCAAATCAGCCC |
| | | CCTGTGAGC |
| 27657 | 42 | EPKSSDKTHTCPPCPAPEAAGGPSVFLFPPKPKDTLMISRTPEV |
| | | TCVVVSVSHEDPEVKFNWYVDGVEVHNAKTKPREEQYNSTY |
| | | RVVSVLTVLHQDWLNGKEYKCKVSNKALPAPIEKTISKAKGQ |
| | | PREPQVYVLPPSRDELTKNQVSLLCLVKGFYPSDIAVEWESNG |
| | | QPENNYLTWPPVLDSDGSFFLYSKLTVDKSRWQQGNVFSCSV |
| | | MHEALHNHYTQKSLSLSPGGGGGSGGGGSGGGGSQSTIEEQA |
| | | KTFLDKFNHEAEDLFYQSSLASWNYNTNITEENVQNMNNAG |
| | | DKWSAFLKEQSTLAQMYPLQEIQNLTVKLQLQALQQNGSSV |
| | | LSEDKSKRLNTILNTMSTIYSTGKVCNPDNPQECLLLEPGLNEI |
| | | MANSLDYNERLWAWESWRSEVGKQLRPLYEEYVVLKNEMA |
| | | RANHYEDYGDYWRGDYEVNGVDGYDYSRGQLIEDVEHTFE |
| | | EIKPLYEHLHAYVRAKLMNAYPSYISPIGCLPAHLLGDMWGR |
| | | FWTNLYSLTVPFGQKPNIDVTDAMVDQAWDAQRIFKEAEKF |
| | | FVSVGLPNMTQGFWENSMLTDPGNVQKAVCHPTAWDLGKG |
| | | DFRILMCTKVTMDDFLTAHNEMGNIQYDMAYAAQPFLLRNG |
| | | ANEGFHEAVGEIMSLSAATPKHLKSIGLLSPDFQEDNETEINFL |
| | | LKQALTIVGTLPFTYMLEKWRWMVFKGEIPKDQWMKKWWE |
| | | MKREIVGVVEPVPHDETYCDPASLFHVSNDYSFIRYYTRTLY |
| | | QFQFQEALCQAAKHEGPLHKCDISNSTEAGQKLFNMLRLGKS |
| | | EPWTLALENVVGAKNMNVRPLLNYFEPLFTWLKDQNKNSFV |
| | | GWSTDWSPYADQSIKVRISLKSALGDKAYEWNDNEMYLFRS |
| | | SVAYAMRQYFLKVKNQMILFGEEDVRVANLKPRISFNFFVTA |
| | | PKNVSDIIPRTEVEKAIRMSRSRINDAFRLNDNSLEFLGIQPTLG |
| | | PPNQPPVS |
| | 43 | GAGCCAAAGAGCTCCGACAAGACCCACACATGCCCCCCTT |
| | | GTCCGGCGCCAGAGGCAGCAGGAGGACCAAGCGTGTTCCT |
| | | GTTTCCACCCAAGCCCAAAGACACCCTGATGATTAGCCGA |
| | | ACCCCTGAAGTCACATGCGTGGTCGTGTCCGTGTCTCACGA |
| | | GGACCCAGAAGTCAAGTTCAACTGGTACGTGGATGGCGTC |
| | | GAGGTGCATAATGCCAAGACAAAACCCCGGGAGGAACAGT |
| | | ACAACAGCACCTATAGAGTCGTGTCCGTCCTGACAGTGCTG |
| | | CACCAGGATTGGCTGAACGGCAAGGAATATAAGTGCAAAG |
| | | TGTCCAATAAGGCCCTGCCCGCTCCTATCGAGAAAACCATT |
| | | TCTAAGGCAAAAGGCCAGCCTCGCGAGCCTCAGGTGTACG |
| | | TGCTGCCACCTTCCCGCGACGAGCTGACCAAGAATCAGGT |
| | | GTCTCTGCTGTGCCTGGTGAAGGGCTTCTATCCAAGCGATA |
| | | TCGCAGTGGAGTGGGAGTCCAACGGACAGCCCGAGAACAA |
| | | TTACCTGACCTGGCCACCCGTGCTGGACAGCGATGGCTCCT |
| | | TCTTTCTGTATTCCAAGCTGACAGTGGACAAGTCTAGATGG |
| | | CAGCAGGGCAACGTGTTCAGCTGTTCCGTGATGCACGAGG |
| | | CCCTGCACAACCACTACACCCAGAAGTCTCTGAGCTTAAGC |
| | | CCTGGAGGAGGAGGAGGCTCCGGCGGAGGAGGCTCTGGA |
| | | GGAGGAGGCAGCCAGTCCACAATCGAGGAGCAGGCCAAG |
| | | ACCTTCCTGGACAAGTTTAACCACGAGGCCGAGGATCTGTT |
| | | CTATCAGAGCTCCCTGGCCAGCTGGAACTACAATACCAAC |
| | | ATCACAGAGGAGAATGTGCAGAACATGAACAATGCCGGGG |
| | | ACAAGTGGTCTGCCTTTCTGAAGGAGCAGAGCACACTGGC |
| | | CCAGATGTACCCACTGCAGGAGATCCAGAACCTGACCGTG |
| | | AAGCTGCAGCTGCAGGCCCTGCAGCAGAATGGCTCTAGCG |
| | | TGCTGTCTGAGGATAAGAGCAAGCGGCTGAATACAATCCT |
| | | GAACACCATGTCCACAATCTATTCTACCGGCAAGGTGTGCA |
| | | ATCCCGACAACCCTCAGGAGTGTCTGCTGCTGGAGCCTGGC |
| | | CTGAATGAGATCATGGCCAACTCCCTGGATTACAATGAGA |
| | | GGCTGTGGGCATGGGAGTCTTGGCGCAGCGAAGTGGGCAA |
| | | GCAGCTGCGGCCACTGTACGAGGAGTATGTGGTGCTGAAG |
| | | AACGAGATGGCCAGAGCCAATCACTACGAGGACTATGGCG |
| | | ATTACTGGAGGGGCGACTACGAGGTGAACGGCGTGGACGG |
| | | CTACGATTATAGCCGGGGCCAGCTGATCGAGGATGTGGAG |
| | | CACACATTCGAGGAGATCAAGCCCCTGTATGAGCACCTGC |
| | | ACGCCTACGTGCGGGCCAAGCTGATGAACGCCTATCCATC |
| | | CTACATCTCTCCAATCGGATGCCTGCCTGCCCACCTGCTGG |
| | | GCGACATGTGGGGCAGATTCTGGACAAACCTGTACTCCCT |
| | | GACCGTGCCATTTGGCCAGAAGCCCAATATCGACGTGACC |
| | | GATGCCATGGTGGACCAGGCCTGGGATGCCCAGAGGATCT |
| | | TCAAGGAGGCCGAGAAGTTCTTTGTGAGCGTGGGCCTGCC |
| | | AAACATGACACAGGGCTTTTGGGAGAACAGCATGCTGACC |
| | | GACCCAGGCAACGTGCAGAAGGCCGTGTGCCACCCAACAG |
| | | CATGGGACCTGGGCAAGGGCGATTTCCGCATCCTGATGTG |
| | | CACCAAGGTGACAATGGACGATTTTCTGACCGCCCACAAT |
| | | GAGATGGGCAACATCCAGTATGATATGGCATACGCAGCAC |
| | | AGCCCTTCCTGCTGCGGAATGGAGCCAACGAGGGCTTTCA |
| | | CGAGGCCGTGGGCGAGATCATGTCTCTGAGCGCCGCCACA |
| | | CCTAAGCACCTGAAGAGCATCGGCCTGCTGTCCCCAGACTT |
| | | CCAGGAGGATAACGAGACCGAGATCAATTTTCTGCTGAAG |
| | | CAGGCCCTGACCATCGTGGGCACACTGCCCTTCACCTATAT |
| | | GCTGGAGAAGTGGCGCTGGATGGTGTTTAAGGGCGAGATC |
| | | CCTAAGGACCAGTGGATGAAGAAGTGGTGGGAGATGAAGC |
| | | GGGAGATCGTGGGCGTGGTGGAGCCTGTGCCACACGACGA |
| | | GACCTACTGTGATCCCGCCAGCCTGTTCCACGTGAGCAACG |
| | | ACTATTCCTTTATCAGGTACTATACCCGCACACTGTACCAG |
| | | TTCCAGTTTCAGGAGGCCCTGTGCCAGGCAGCAAAGCACG |
| | | AGGGCCCACTGCACAAGTGTGATATCTCCAACTCTACAGA |
| | | GGCCGGACAGAAGCTGTTCAATATGCTGAGACTGGGCAAG |
| | | TCCGAGCCATGGACCCTGGCCCTGGAGAACGTGGTGGGAG |
| | | CCAAGAATATGAACGTGAGGCCTCTGCTGAATTATTTCGAG |
| | | CCACTGTTTACATGGCTGAAGGATCAGAATAAGAACTCTTT |
| | | CGTGGGCTGGTCTACCGACTGGAGCCCTTACGCCGATCAG |
| | | AGCATCAAGGTGAGAATCAGCCTGAAGTCCGCCCTGGGCG |
| | | ACAAGGCCTATGAGTGGAATGATAACGAGATGTACCTGTT |
| | | CAGATCCTCTGTGGCCTATGCCATGAGGCAGTACTTCCTGA |
| | | AGGTGAAGAACCAGATGATCCTGTTTGGCGAGGAGGACGT |
| | | GCGGGTGGCCAATCTGAAGCCCAGAATCTCCTTTAACTTCT |
| | | TTGTGACAGCCCCCAAGAACGTGAGCGATATCATCCCTCGC |
| | | ACCGAGGTGGAGAAGGCCATCCGGATGAGCCGGTCCAGAA |
| | | TCAACGACGCCTTCAGGCTGAATGATAACTCCCTGGAGTTT |
| | | CTGGGCATCCAGCCTACCCTGGGCCCACCTAATCAGCCACC |
| | | CGTGTCT |

1.-54. (canceled)

55. A polypeptide construct, comprising:
(i) at least three binding domains, each binding domain comprising an ACE2 protein capable of binding to a viral spike protein;
(ii) at least three dimeric IgM Fc domains, each dimeric IgM Fc domain comprising a first Fc polypeptide and a second Fc polypeptide, the first and the second Fc polypeptides each comprising an IgM CH2 sequence, an IgM CH3 sequence, and an IgM CH4 sequence comprising a tailpiece sequence; and
(iii) a tailpiece assembly,
wherein:
a) each Fc polypeptide of each dimeric IgM Fc domain is coupled via its N-terminus to one of the at least three binding domains, and
b) the tailpiece sequences of the Fc polypeptides form the tailpiece assembly.

56. The polypeptide construct of claim 55, comprising four, five or six dimeric IgM Fc domains.

57. The polypeptide construct of claim 55, comprising five dimeric IgM Fc domains and ten binding domains, wherein each binding domain is coupled to the N-terminus of a first or second Fc polypeptide of the five dimeric IgM Fc domains, thereby forming a decavalent polypeptide construct.

58. The polypeptide construct of claim 55, wherein at least one of the binding domains comprises an ACE2 protein comprising an amino acid sequence having at least about 80%, 90%, 95%, 97%, 99%, or 100% sequence identity to any of the amino acid sequences set forth in SEQ ID NOs: 1-3.

59. The polypeptide construct of claim 55, wherein each binding domain comprises an ACE2 protein comprising an amino acid sequence having at least about 80%, 90%, 95%, 97%, 99%, or 100% sequence identity to any of the amino acid sequences set forth in SEQ ID NOs: 1-3.

60. The polypeptide construct of claim 55, wherein at least one of the binding domains comprises an ACE2 protein comprising one or more amino acid substitutions relative to any of the amino acid sequences set forth in SEQ ID NOs: 1-3.

61. The polypeptide construct of claim 60, wherein the one or more amino acid substitutions comprise the catalytic knock-out (KO) mutations H374N_H378N relative to the sequence set forth in SEQ ID NO: 1, resulting in a catalytic KO sequence of the extracellular domain of ACE2 set forth in SEQ ID NO: 4.

62. The polypeptide construct of claim 60, wherein the one or more amino acid substitutions comprise substitution(s) that increase the stability of the binding domain relative to a binding domain comprising an ACE2 protein without the one or more amino acid substitutions, and wherein the increase in stability is measured as an increase in melting temperature of at least about 1° C., 2° C., 3° C., 4° C., 5° C., 6° C., 7° C., or 8° C. as measured using differential scanning calorimetry (DSC).

63. The polypeptide construct of claim 62, wherein the binding domain with the one or more amino acid substitutions comprises an amino acid sequence selected from any one of SEQ ID NOs: 44-72.

64. The polypeptide construct of claim 55, wherein (i) the IgM CH2 sequence comprises an amino acid sequence having at least about 80%, 90%, 95%, 97%, 99%, or 100% sequence identity to the amino acid sequence set forth in SEQ ID NO: 5, (ii) the IgM CH3 sequence comprises an amino acid sequence having at least about 80%, 90%, 95%, 97%, 99%, or 100% sequence identity to the amino acid sequence set forth in SEQ ID NO: 6, and (iii) the IgM CH4 sequence comprises an amino acid sequence having at least about 80%, 90%, 95%, 97%, 99%, or 100% sequence identity to the amino acid sequence set forth in SEQ ID NO: 7.

65. The polypeptide construct of claim 55, wherein each Fc polypeptide of each dimeric IgM Fc domain is coupled via its N-terminus to one of the at least three binding domains with or without a linker.

66. The polypeptide construct of claim 55, further comprising a J-chain.

67. The polypeptide construct of claim 66, wherein the J-chain comprises an amino acid sequence having at least about 80%, 90%, or 100% sequence identity to the amino acid sequence set forth in SEQ ID NO: 10 or SEQ ID NO: 12.

68. The polypeptide construct of claim 55, comprising at least one fusion polypeptide comprising an amino acid sequence having at least about 80%, 90%, 95%, 97%, 99%, or 100% sequence identity to the amino acid sequence set forth in SEQ ID NO: 28 or SEQ ID NO: 30.

69. The polypeptide construct of claim 55, wherein the polypeptide construct comprises (i) a fusion polypeptide with the sequence set forth in SEQ ID NO: 28 and a J-chain with the sequence set forth in SEQ ID NO: 10, or (ii) a fusion polypeptide with the sequence set forth in SED ID NO: 30 and a J-chain with the sequence set forth in SEQ ID NO: 10.

70. The polypeptide construct of claim 55, wherein the viral spike protein is derived from a SARS associated virus.

71. The polypeptide construct of claim 70, wherein the SARS associated virus is SARS-coronavirus (SARS-CoV) or SARS-coronavirus 2 (SARS-CoV-2).

72. The polypeptide construct of claim 55, wherein the polypeptide construct has an apparent K_{D} for binding the viral spike protein of ≤10⁻⁹ M or ≤10⁻¹⁰ M.

73. A pharmaceutical composition comprising the polypeptide construct of claim 55 and a pharmaceutically acceptable carrier or excipient.

74. The pharmaceutical composition of claim 73, formulated for intranasal, oral, parenteral, or pulmonary administration.

75. A nucleic acid molecule or a set of nucleic acid molecules encoding the polypeptide construct of claim 1.

76. A host cell comprising the nucleic acid molecule or the set of nucleic acid molecules of claim 75.

77. A method of producing the polypeptide construct of claim 55, the method comprising:
(a) expressing the polypeptide construct in a host cell of a host cell culture, the host cell comprising a nucleic acid molecule or a set of nucleic acid molecules encoding the polypeptide construct, and
(b) recovering the polypeptide construct from the host cell culture.

78. A method of treating a viral infection in a subject in need thereof, the method comprising:
administering to the subject an effective amount of a polypeptide construct, wherein the polypeptide construct comprises:
(i) at least three binding domains, each binding domain comprising an ACE2 protein capable of binding to a viral spike protein;
(ii) at least three dimeric IgM Fe domains, each dimeric IgM Fc domain comprising a first Fe polypeptide and a second Fc polypeptide, the first and the second Fc polypeptides each comprising an IgM CH2 sequence, an IgM CH3 sequence, and an IgM CH4 sequence comprising a tailpiece sequence; and
(iii) a tailpiece assembly;
wherein,
a) each Fc polypeptide of each dimeric IgM Fc domain is coupled via its N-terminus to one of the at least three binding domains, and
b) the tailpiece sequences of the Fc polypeptides form the tailpiece assembly; and
thereby treating the viral infection in the subject.

79. The method of claim 78, wherein administering the polypeptide construct to the subject comprises intranasal, oral, parenteral, or pulmonary administration.

80. The method of claim 78, wherein the viral infection is caused by a SARS associated virus, optionally a SARS-CoV associated virus or a SARS-CoV-2 associated virus.
